SEQUENCE LISTING (9 sequences)

SEQ ID NO: 1 - protein, 5 aa, Artificial Sequence
  siplizumab VH CDR1
EYYMY

SEQ ID NO: 2 - protein, 17 aa, Artificial Sequence
  siplizumab VH CDR2
RIDPEDGSIDYVEKFKK

SEQ ID NO: 3 - protein, 9 aa, Artificial Sequence
  siplizumab VH CDR3
GKFNYRFAY

SEQ ID NO: 4 - protein, 16 aa, Artificial Sequence
  siplizumab VL CDR1
RSSQSLLHSSGNTYLN

SEQ ID NO: 5 - protein, 7 aa, Artificial Sequence
  siplizumab VL CDR2
LVSKLES

SEQ ID NO: 6 - protein, 9 aa, Artificial Sequence
  siplizumab VL CDR3
MQFTHYPYT

SEQ ID NO: 7 - protein, 118 aa, Artificial Sequence
  Ab1 a Heavy chain variable region
QVQLVQSGAEVQRPGASVKVSCKASGYIFTEYYMYWVRQAPGQGLELVGRIDPEDGSIDYVEKFKKKVTLTADTSSSTAYMELSSLTSDDTAVYYCARGKFNYRFAYWGQGTLVTVSS

SEQ ID NO: 8 - protein, 112 aa, Artificial Sequence
  Ab1 light chain variable region
DVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIK

SEQ ID NO: 9 - protein, 118 aa, Artificial Sequence
  Ab1 Heavy chain variable region
QVQLVQSGAEVKKPGASVKVSCKASGYTFTEYYMYWVRQAPGQGLELMGRIDPEDGSIDYVEKFKKKVTLTADTSSSTAYMELSSLTSDDTAVYYCARGKFNYRFAYWGQGTLVTVSS

(19) United States
(12) Patent Application Publication
(10) Pub. No.: US 2024/0101671 A1
(43) Pub. Date: Mar. 28, 2024

(54) METHOD AND COMPOSITION FOR INDUCING TOLERANCE

(71) Applicant: Zelarion Malta Limited, BIRKIRKARA (MT)

(72) Inventor: Erik BERGLUND, New York, NY (US)

(21) Appl. No.: 18/273,725

(22) PCT Filed: Jan. 20, 2022
(86) PCT No.: PCT/EP2022/051283

§ 371 Date: Jul. 21, 2023

Related U.S. Application Data
(60) Provisional application No. 63/141,023, filed on Jan. 25, 2021.

(51) Int. Cl. C07K 16/28; A61K 31/436; A61K 31/5355; A61K 31/573; A61K 31/675; A61K 35/407; A61K 39/395; A61P 37/06

(52) U.S. Cl. CPC C07K 16/2806; A61K 31/436; A61K 31/5355; A61K 31/573; A61K 31/675; A61K 35/407; A61K 39/3955; A61P 37/06; A61K 2039/505; C07K 2317/24; C07K 2317/565

(57) ABSTRACT

Provided herein are methods for inducing tolerance to an organ (e.g., liver) transplant in a patient. Compositions for use with these methods and kits are also disclosed.

### 1. CROSS-REFERENCE TO RELATED APPLICATIONS

This application claims the benefit of U.S. Ser. No. 63/141,023 filed Jan. 25, 2021, the disclosure of which is incorporated by reference herein in its entirety.

### 2. REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Jan. 20, 2022, is named 105848PC_SeqListing.txt and is 5,000 bytes in size.

### 3. FIELD OF THE INVENTION

Provided herein are methods for inducing tolerance to an organ (e.g., liver) transplant in a patient. Compositions for use with these methods and kits are also disclosed.

### 4. BACKGROUND

Long-term outcomes in solid organ transplantation, including liver transplantation, remain unsatisfactory. Chronic rejection of the donor graft and health issues associated with long-term immunosuppressant use continue to be serious complications of the procedure. These complications, however, could be overcome by inducing tolerance to the donor graft by the recipient's immune system. Tolerance avoids a destructive immune response following transplantation without the need for systemic immunosuppression.

The contemporary standard of care transplant regimen post liver transplantation is most frequently based on the calcineurin inhibitor tacrolimus in combination with the antiproliferative agent mycophenolic acid (mycophenolate) which blocks inosine monophosphate dehydrogenase. mTOR inhibitors such as sirolimus (rapamycin) or everolimus may also be used. In addition to the calcineurin inhibitor and antiproliferative agent and corticosteroids are usually started in the peri-transplant period and continued post-transplant in a triple therapy standard of care regimen. The most recent data (Kwong et al. (2020), Am J Transplant. 20 Suppl sl:193-299. doi: 10.1111/ajt.15674. PMID: 31898413) showed that 62.3% of liver recipients were taking a combination of tacrolimus, mycophenolate, and steroids, 18.6% were taking tacrolimus and mycophenolate and 5.6% were taking tacrolimus and steroids with 12.8% taking other regimens. In contrast to renal transplantation, induction agents were not used in the majority of liver transplants (69.4%) with 22.0% using interleukin-2 (IL-2) receptor antagonists and 8.9% using T-cell depleting agents.

Although initial outcomes of liver transplantation are good, long-term allograft survival with current immunosuppression is suboptimal; 55.0% and 57.4% at ten years post-transplant for European (Adam et al. (2018), Transpl. Int., 31, 1293-1317, doi:10.1111/tri.13358) and US (Kwong et al. (2020), Am J Transplant. 20 Suppl sl:193-299. doi: 10.1111/ajt.15674. PMID: 31898413) for deceased donor transplant populations respectively. The side effects of long-term immunosuppression, especially renal impairment, cardiovascular, neurological and infectious comorbidities, combined with chronic rejection, all negatively impact long term liver transplant outcomes.

Thus, there is an unmet need for tolerance approaches that reduce or eliminate the comorbidities of immunosuppression and thereby improve patient and allograft survival. Clinical spontaneous operational liver allograft tolerance (defined as discontinuation of all immunosuppression for one year while maintaining stable allograft status) in multicenter studies is presented as possible in 13-40% (Benitez et al. (2013), Hepatology 2013, 58, 1824-1835, doi:10.1002/hep.26426) and 38-60% (Feng S et al. (2012), JAMA 307, 283-293, doi:10.1001/jama.2011.2014); Bohne et al. (2012), J. Clin. Invest. 122, 368-382, doi:10.1172/JCI59411) of adult and pediatric liver transplant recipients, respectively. However, these seemingly encouraging numbers are from groups of highly selected recipients with stable blood chemistries and clean liver biopsies for several years post-transplant (Feng et al. (2017), Hepatology 65, 647-660, doi:10.1002/hep.28681; Benitez et al. (2013), Hepatology 2013, 58, 1824-1835, doi:10.1002/hep.26426). Notably, the probability of successful immunosuppression withdrawal is higher the longer after the liver transplantation that withdrawal is intentionally attempted. The limited success of standard immunosuppression withdrawal early post-transplant, the high accumulated immunosuppression exposure and associated side-effects, the relatively small subset of the overall liver transplant patients that can be weaned off immunosuppression (around 15%), and evidence of ongoing host allo-immune responses even in tolerant patients (Feng et al. (2017), Hepatology 65, 647-660, doi:10.1002/hep.28681; Ohe et al. (2014), Transplantation 98, 1105-1111, doi:10.1097/TP.0000000000000185; Shaked et al. (2019), Am J Transplant. 2019 May; 19(5):1397-1409. doi: 10.1111/ajt.15205), suggests that rapid tolerance induction is the best approach to protect liver allografts from immune-mediated damage while eliminating the significant side effects of immunosuppressive medications. These studies show that in a small number of highly selected patients, having normal chemistries and follow-up biopsies, at a significant time post-transplant that immunosuppression, withdrawal can be performed. Therefore, an approach which can generate tolerance in the broad population of liver transplant recipients is required.

Human CD2 (also known as LFA-2, for leukocyte function-associated antigen-2) is a monomeric transmembrane glycoprotein of 45-50 kDa. CD2 is expressed early during human thymocyte development and is found on about half of thymocytes, thymic B-cells, natural killer (NK) cells and almost all mature peripheral T-cells. CD2 functions as an intercellular adhesion molecule, binding to its ligand LFA-3 (CD58) in humans with high affinity. The CD2-CD58 complex functions as an intercellular adhesion complex, forming a conjugate and induced a conformation change. The formation of the conjugate gives the T-cell receptor (TCR) a longer interval to scan various peptide-major histocompatibility complex (pMHC) combinations presented by the antigen-presenting cells (APCs), determine if a match has been made, and, if so, complete the intracellular signaling and co-stimulation necessary for T-cell activation (Springer et al. (1987), Annu Rev Immunol 5:223-52.10.1146/annurev.iy.05.040187.001255; Davis et al. (1996), Immunol Today 17:177-87.10.1016/0167-5699(96)80617-7; Seed et al. (1987), Proc Natl Acad Sci USA 84:3365-9.10.1073/pnas.84.10.3365; Binder et al. (2020), Front. Immunol. 11:1090.doi: 10.3389/fimmu.2020.01090).

The interaction of CD58 with CD2 has been found to be essential for the activation of cellular immunity, such as CD8⁺ cytotoxic T-lymphocytes and NK cell-mediated cytotoxic reactions (Rolle et al. (2016), Eur J Immunol 46:2420-5.10.1002/eji.201646492; Leitner J et al. (2015), J Immunol 195:477-87.10.4049/jimmunol.1401917). In addition, CD2 is up-regulated on both activated and memory T-cells while CD58-ligation to CD2 activates NK and dendritic cells, lowers the threshold for T-cell activation and enhances T-cell responsiveness to pro-inflammatory cytokines such as IL-12 (Lo et al. (2011), Am J Transplant. 11(1):22-33. Doi: 10.1111/j.1600-6143.2010.03317.x). Furthermore, CD2-ligation of B-cell specific CD58 induces the upregulation of CD40 expression, suggesting that CD2 may also play a role in the stimulation and/or delivery of T-cell help to B-cells.

Siplizumab (TCD601) is a non-agonistic, humanized, anti-CD2 monoclonal antibody of the IgG1κ class. Siplizumab binds to a unique epitope on human CD2, distinct from the CD58 binding site, with high affinity (kd—5 nM), inhibiting co-stimulation and T-cell activation. In addition, the Fc portion of the siplizumab antibody binds to FcγR receptors on NK cells resulting in ADCC and antibody-dependent cellular phagocytosis (ADCP) mediated depletion of CD2+ lymphocytes. Siplizumab also demonstrates selective immunomodulatory activity with depletion of memory T-cells (Tmem; high CD2 expression) and sparing of regulatory T-cells (Treg; low CD2 expression) in vitro and in vivo due to the differential expression of CD2. Considering this activity, siplizumab is expected to modulate T-cell memory and immune reactivity in the setting of transplantation.

### 5. SUMMARY

Provided herein is a method of transplantation, the method comprising (i) transplanting a donor liver into a human recipient; and (ii) administering to the recipient an anti-CD2 antibody. In some embodiments, the anti-CD2 antibody comprises (a) a heavy chain variable region CDR 1 of SEQ ID NO:1; (b) a heavy chain variable region CDR 2 of SEQ ID NO:2; (c) a heavy chain variable region CDR 3 of SEQ ID NO:3; (d) a light chain variable region CDR 1 of SEQ ID NO:4; (e) a light chain variable region CDR 2 of SEQ ID NO:5; and (d) a light chain variable region CDR 3 of SEQ ID NO:6. In some embodiments, the antibody is a humanized antibody. In some embodiments, the antibody is siplizumab.

In some embodiments, the anti-CD2 antibody is administered to the recipient at least once within two weeks after the transplant. In some embodiments, the anti-CD2 antibody is administered to the recipient on the day of the transplant, on day 1 after the transplant and on day 4 after the transplant. In some embodiments, the anti-CD2 antibody is administered to the recipient at a dose of 0.1-5 mg/kg/dose. In some embodiments, the anti-CD2 antibody is administered to the recipient at a dose of about 0.6 mg/kg/dose. In some embodiments, the anti-CD2 antibody is administered to the recipient intravenously or subcutaneously.

In some embodiments, the recipient has undergone a splenectomy. In some embodiments, the recipient has not undergone a splenectomy.

In some aspects, a method of treatment provided herein method further comprises administering cyclophosphamide to the recipient. In some embodiments, the cyclophosphamide is administered to the recipient at least once during the 30 days after the transplant. In some embodiments, the cyclophosphamide is administered to the recipient on day 5 after the transplant. In some embodiments, the cyclophosphamide is administered to the recipient at a dose of 20-100 mg/kg/dose. In some embodiments, the cyclophosphamide is administered to the recipient at a dose of about 40 mg/kg/dose. In some embodiments, the cyclophosphamide is administered to the recipient intravenously.

In some embodiments, a method of treatment provided herein further comprises administering a calcineurin inhibitor to the recipient. In some embodiments, the calcineurin inhibitor is administered to the recipient once day or twice a day. In some embodiments, the calcineurin inhibitor is administered to the recipient within 24 hours of liver reperfusion. In some embodiments, the calcineurin inhibitor is administered to the recipient for at least 1 month, 2 months, 3 months, 4 months, 5 months, 6 months, 7 months, 8 months, 9 months, 10 months, 11 months, 12 months, 13 months, or at least 14 months post-transplant. In some embodiments, the calcineurin inhibitor is administered to the recipient at an amount sufficient to maintain serum through concentrations in the range of 2-15 ng/mL. In some embodiments, the calcineurin inhibitor is administered to the recipient at an amount sufficient to maintain serum through concentrations in the range of 4-11 ng/mL. In some embodiments, the calcineurin inhibitor is administered to the recipient orally. In some embodiments, the calcineurin inhibitor is administered to the recipient intravenously.

In some embodiments, the recipient is gradually weaned off the calcineurin inhibitor over 3-24 months after transplantation. In some embodiments, the recipient is gradually weaned off the calcineurin inhibitor over 6-18 months after transplantation. In some embodiments, the dose of the calcineurin inhibitor is reduced to three quarters of the daily dose once a day at six months after transplantation. In some embodiments, the dosing frequency of the calcineurin inhibitor is reduced three times a week at nine months after transplantation. In some embodiments, the dosing frequency of the calcineurin inhibitor is further reduced twice a week at twelve months after transplantation. In some embodiments, the dosing frequency of the calcineurin inhibitor is further reduced to once a week at 15 months after transplantation. In some embodiments, the administration of the calcineurin inhibitor is stopped 18 months after transplantation. In some embodiments, the calcineurin inhibitor is tacrolimus.

In some aspects, a method of treatment provided herein further comprises administering steroids to the recipient. In some embodiments, the steroid is prednisone. In some embodiments, the steroid is administered to the recipient on the day of the transplant. In some embodiments, the steroid is administered at a dose of 500-1500 mg. In some embodiments, the steroid is administered at a dose of 1000 mg. In some embodiments, the steroid is administered from day 2 after the transplant through three months after the transplant. In some embodiments, the steroid is administered from day 2 after the transplant through one month after the transplant. In some embodiments, the steroid is administered orally at a dose of 250 mg/day on day 2, a dose of 125 mg/day on day 3, a dose of 75 mg/day on day 4, and at a dose of 60 mg/day from day 5 through to one month after transplant.

In some aspects, a method of treatment provided herein further comprises administering standard-of-care antimetabolite therapy to the recipient. In some embodiments, the standard-of-care antimetabolite therapy is administered twice a day for a daily dose of 500-1500 mg/day. In some embodiments, the standard-of-care antimetabolite therapy is administered no later than 24 hours after graft reperfusion. In some embodiments, the standard-of-care antimetabolite therapy administration is stopped within six months post-transplant. In some embodiments, the standard-of-care antimetabolite therapy administration is stopped one month post-transplant. In some embodiments, standard-of-care antimetabolite therapy is mycophenolate.

### 6. BRIEF DESCRIPTION OF THE FIGURES

FIG. 1 shows an exemplary design of a clinical trial to evaluate the treatments described herein, e.g., the clinical trial described in Example 1.

### 7. DETAILED DESCRIPTION OF THE INVENTION

Described herein are methods for the induction of immunological tolerance of a liver transplant recipient's immune system towards the transplanted organ (e.g., liver) without the need for ongoing immunosuppressive therapy. Specifically, provided herein are methods for inducing tolerance to a transplanted organ (e.g., liver) using a combination of an anti-CD2 antibody, cyclophosphamide. In some embodiments, the method for inducing tolerance further comprises administering a splenectomy. In certain embodiments, the components of the regimen can be modified as described herein to achieve induction of tolerance of an organ transplant recipient's immune system towards the transplanted organ without the need for ongoing immunosuppressive therapy. In other embodiments, the components of the regimen can be modified as described herein to achieve induction of tolerance of an organ transplant recipient's immune system towards the transplanted organ without the need for splenectomy.

Recipients treated in accordance with the methods provided herein are described in Section 7.1. Transplants and donors are described in 7.2. Postoperative treatment regimens are described in Section 7.3. Methods to assess clinical outcomes are described in Section 7.4.

In a specific embodiment, an individual who has been selected to receive an organ (e.g., liver) transplant may be treated using the methods described herein. In a specific embodiment, a patient will receive a liver transplant and splenectomy and will then be treated with a standard-of-care immunosuppressive combination regimen, e.g. a calcineurin inhibitor (e.g., tacrolimus), standard-of-care antimetabolite (e.g. mycophenolate) therapy, with or without steroids. Use of other standard-of-care combination regimens which may include cyclosporine or sirolimus or everolimus or azathioprine may also be used as initial immunosuppression.

In specific embodiments, a liver transplant recipient who has undergone a splenectomy is treated with (i) 0.6 mg/kg Siplizumab given intravenously on the day of the liver transplant (Day 0), as well as on Days 1 and 4; (ii) 40 mg/kg cyclophosphamide given intravenously on Day 5; (iii) tacrolimus given orally twice a day at a dose sufficient to maintain serum trough concentrations of 4-11 ng/mL; (iv) 250-750 mg mycophenolate given orally twice daily until month 1; and (v) steroids administered at 2×500 mg immediately pre and post liver transplant and rapidly tapered down to 20 mg/day then stopped at Month 1. This treatment regimen can be modified as described below. This treatment regimen can be modified as described below. Specifically, route of administration, dose, and exact timing of the various active pharmaceutical ingredients can be modified to adjust to the specific circumstances of the transplantation to achieve induction of tolerance of an organ (e.g., liver) transplant recipient's immune system towards the transplanted organ (e.g., liver) without the need for ongoing immunosuppressive therapy.

### 7.1 Recipients and Indications

Individuals whose liver has been damaged by means including injury, disease, or birth defect may meet the criteria to receive an organ (e.g., liver) transplant and may be treated in accordance with the methods described herein. A recipient treated in accordance with the methods described herein may have required a liver transplant for any reason. Generally, a patient suffering from end-stage liver disease whose life expectancy is predicted to be extended by a liver transplant beyond the life expectancy without the liver transplant may be considered for a liver transplant. Examples of indications of a liver transplant are described, for example, in the EASL Clinical Practice Guidelines: Liver transplantation (J Hepatol. 2016 February; 64(2):433-485. doi: 10.1016/j.jhep.2015.10.006). In specific embodiments, a recipient treated in accordance with the methods described herein has received a liver transplant necessitated by cirrhosis of the liver.

In specific embodiments, a recipient treated in accordance with the methods described herein has received a liver transplant necessitated by a hepatic cancer, such as fibrolamellar carcinoma or epitheliod hemangioendothelioma. In specific embodiments, a recipient treated in accordance with the methods described herein has received a liver transplant necessitated by hepatocellular carcinoma. In specific embodiments, a recipient treated in accordance with the methods described herein has received a liver transplant necessitated by choloangiocarcinoma. In specific embodiments, a recipient treated in accordance with the methods described herein has received a liver transplant necessitated by hepatic metastases of a cancer.

In specific embodiments, a recipient treated in accordance with the methods described herein has received a liver transplant necessitated by acute liver failure. The acute liver failure may be due to any cause including, for example, infection with Hepatitis A virus, Hepatitis B virus, Hepatitis C virus, or exposure to toxic agents, alcohol, or drugs.

In specific embodiments, a recipient treated in accordance with the methods described herein has received a liver transplant necessitated by Hepatitis B-related liver disease. In specific embodiments, a recipient treated in accordance with the methods described herein has received a liver transplant necessitated by Hepatitis C-related liver disease. In specific embodiments, a recipient treated in accordance with the methods described herein has received a liver transplant necessitated by alcoholic liver disease.

In specific embodiments, a recipient treated in accordance with the methods described herein has received a liver transplant necessitated by non-alcoholic fatty liver disease (NAFLD). In specific embodiments, a recipient treated in accordance with the methods described herein has received a liver transplant necessitated by non-alcoholic steatohepatitis (NASH). In specific embodiments, a recipient treated in accordance with the methods described herein has received a liver transplant necessitated by primarily biliary cholangitis (PBC). In specific embodiments, a recipient treated in accordance with the methods described herein has received a liver transplant necessitated by autoimmune hepatitis (AIH).

In specific embodiments, a recipient treated in accordance with the methods described herein has received a liver transplant necessitated by a genetic disease, such as genetic cholestatic disorders, Wilson's disease, hereditary haemochromatosis, tyrosinemia, alpha-1-antitrypsin deficiency, urea cycle disorders, Crigler-Najjar syndrome, familial amyloid polyneuropathy, primary hyperoxaluria type 1, or atypical hemolytic uremic syndrome-1. In some embodiments, a recipient treated in accordance with the methods described herein has received a liver transplant necessitated by a genetic cholestatic disorder. In some embodiments, a recipient treated in accordance with the methods described herein has received a liver transplant necessitated by Wilson's disease. In some embodiments, a recipient treated in accordance with the methods described herein has received a liver transplant necessitated by hereditary haemochromatosis. In some embodiments, a recipient treated in accordance with the methods described herein has received a liver transplant necessitated by tyrosinemia, alpha-1-antitrypsin deficiency. In some embodiments, a recipient treated in accordance with the methods described herein has received a liver transplant necessitated by an urea cycle disorder. In some embodiments, a recipient treated in accordance with the methods described herein has received a liver transplant necessitated by Crigler-Najjar syndrome. In some embodiments, a recipient treated in accordance with the methods described herein has received a liver transplant necessitated by familial amyloid polyneuropathy. In some embodiments, a recipient treated in accordance with the methods described herein has received a liver transplant necessitated by, primary hyperoxaluria type 1. In some embodiments, a recipient treated in accordance with the methods described herein has received a liver transplant necessitated by atypical hemolytic uremic syndrome-1.

In specific embodiments, the liver transplant is a living donor liver transplant. In specific embodiments, the liver transplant is a deceased donor liver transplant.

In some embodiments, the liver transplant may be an ABO compatible transplant. In some embodiments, the liver transplant may be an ABO incompatible transplant.

In specific embodiments, a recipient treated in accordance with the methods described herein can have a Model for End-Stage Liver Disease (MELD) score of less than 30, less than 20, or less than 10. In some embodiments, a recipient treated in accordance with the methods described herein can have a MELD score of less than 30. In some embodiments, a recipient treated in accordance with the methods described herein can have a MELD score of less than 20. In some embodiments, a recipient treated in accordance with the methods described herein can have a MELD score of less than 10. In some embodiments, a recipient treated in accordance with the methods described herein can be seropositive for Epstein-Barr Virus (EBV).

In specific embodiments, a recipient treated in accordance with the methods described herein does not have end-stage liver disease of autoimmune origin (including autoimmune hepatitis, primary biliary cholangitis or primary sclerosing cholangitis). In specific embodiments, a recipient treated in accordance with the methods described herein does not have leukopenia (defined as white blood cell counts of less than 2,000/mm³) or thrombocytopenia (defined as platelet count of less than 100,000/mm³) at the time of the transplant. In specific embodiments, a recipient treated in accordance with the methods described herein is not seropositive for Human Immunodeficiency Virus 1 (HIV-1) or Hepatitis B surface antigen (HBsAg). In specific embodiments, a recipient treated in accordance with the methods described herein does not have latent tuberculosis (TB) infection as detected by Quantiferon Gold Plus IGRA or interferon gamma release assay. In some embodiments, a recipient treated in accordance with the methods described herein does not have any extrahepatic malignancy or history extrahepatic malignancy other than basal cell carcinoma of the skin or carcinoma in situ of the cervix. In specific embodiments, a recipient treated in accordance with the methods described herein has not received any live-attenuated vaccine within two months of the transplant.

In certain embodiments, the recipient has undergone a splenectomy prior to receiving the transplant. In certain embodiments, the recipient has not undergone a splenectomy prior to receiving the transplant.

The terms “subject,” “recipient,” and “patient” are used interchangeably herein. In certain embodiments, the recipient treated in accordance with a method described herein is human.

### 7.2 Transplant and Donors

As used herein, the donor is the individual from which the organ (e.g., liver) to be transplanted is taken. The donor can be of the same species as the recipient and the donor can be alive or deceased. The donor can be related to the recipient or not related to the recipient. As used herein, the recipient is the individual which will receive the transplanted organ (e.g., liver). The recipient can be related or not related to the donor. The recipient can be HLA-matched or HLA-mismatched with the donor. In specific embodiments, a liver is transplanted from a deceased donor to a recipient.

In some embodiments, the transplanted organ (e.g., liver) may be whole organ, a part of an organ, or cells derived from an organ. In specific embodiments, a whole liver is transplanted. In specific embodiments, only a partial liver is transplanted.

### 7.3 Treatments and Medications

The methods provided herein can include administration of one or more of the treatments below. A recipient treated in accordance with the methods described herein may have undergone a splenectomy before receiving the transplant. Any of the medications described below may be administered to a splenectomized patient.

The procedure for obtaining and implanting the organ (e.g., liver) is well-known to the skilled artisan. Any procedure for the surgical removal from the donor and the surgical implantation in the recipient can be used with the methods provided herein. In certain embodiments, the organ (e.g., liver) can be treated between removal and implantation.

### 7.3.1 Anti-CD2 Antibody

In certain embodiments, the methods described herein include administering an anti-CD2 antibody to the recipient. In specific embodiments, an anti-CD2 antibody for use with the present methods and compositions can have the CDR sequences of rat anti-CD2 monoclonal antibody BTI-322. In specific embodiments, an anti-CD2 antibody can be a humanized IgG1 version of BTI-322 (siplizumab).

In certain embodiments, administration of the anti-CD2 antibody used in the methods described herein does not result in target cell depletion. In certain embodiments, the anti-CD2 antibody used in the methods described herein exhibits immunomodulatory activity. In a specific embodiment, the ability of the anti-CD2 antibody used in the methods described herein to not cause target cell depletion while retaining immunomodulatory activity is accomplished by eliminating glycosylation of Fc region.

In certain embodiments, anti-CD2 antibody used in the methods described herein has no or reduced antibody-dependent cellular cytotoxicity (“ADCC”) activity. Said anti-CD2 antibody can be generated as to exhibit reduced or absent ADCC using methods including, but not limited to, Fc silencing, subclass switching, deglycosylation, and other mutations or modifications of the Fc region. These methods are described, for example, in U.S. Provisional Application No. 63/135,381, which is incorporated herein by reference in its entirety for non-limiting examples of anti-CD2 antibodies that may be used in the methods described herein.

ADCC activity can be determined by any commercially available kit (see, e.g. Promega ADCC Reporter Bioassay, Core Kit (Cat. #G7010, G7018), or any appropriate assay. Such assays can include, but are not limited to, a flow cytometry-based assay, a fluorometric assay, or a bioluminescent reporter assay.

In certain embodiments, the anti-CD2 antibody used in the methods described herein exhibits at most 90% of the ADCC activity of siplizumab in an in vitro assay. An example of such an assay is described in the methods of Golay et al., Haematologica. January 2003; 88:1002-1012. Specifically, the anti-CD2 antibody used in the methods provided herein may exhibit at most 0%, 5%, 10%, 20%, 30%, 40%, 50%, 60%, 70%, 80%, or at most 90% of the ADCC activity as compared to siplizumab. In some embodiments, the anti-CD2 antibody used in the methods provided herein may exhibit no ADCC activity as compared to siplizumab. In some embodiments, the anti-CD2 antibody used in the methods provided herein may exhibit at most 5% of the ADCC activity as compared to siplizumab. In some embodiments, the anti-CD2 antibody used in the methods provided herein may exhibit at most 10% of the ADCC activity as compared to siplizumab. In some embodiments, the anti-CD2 antibody used in the methods provided herein may exhibit at most 20% of the ADCC activity as compared to siplizumab. In some embodiments, the anti-CD2 antibody used in the methods provided herein may exhibit at most 30% of the ADCC activity as compared to siplizumab. In some embodiments, the anti-CD2 antibody used in the methods provided herein may exhibit at most 40% of the ADCC activity as compared to siplizumab. In some embodiments, the anti-CD2 antibody used in the methods provided herein may exhibit at most 50% of the ADCC activity as compared to siplizumab. In some embodiments, the anti-CD2 antibody used in the methods provided herein may exhibit at most 60% of the ADCC activity as compared to siplizumab. In some embodiments, the anti-CD2 antibody used in the methods provided herein may exhibit at most 70% of the ADCC activity as compared to siplizumab. In some embodiments, the anti-CD2 antibody used in the methods provided herein may exhibit at most 80% of the ADCC activity as compared to siplizumab. In some embodiments, the anti-CD2 antibody used in the methods provided herein may exhibit at most 90% of the ADCC activity as compared to siplizumab.

In certain embodiments, in vivo administration of the anti-CD2 antibody used in the methods provided herein exhibits at most 90% of the ADCC activity as compared to in vivo administration of siplizumab in a humanized mouse model or a human subject in a clinical setting. Specifically, in vivo administration of the anti-CD2 antibody used in the methods provided herein may exhibit at most 0%, 5%, 10%, 20%, 30%, 40%, 50%, 60%, 70%, 80%, or at most 90% of the ADCC activity as in vivo administration of siplizumab in a humanized mouse model or a human subject in a clinical setting. In some embodiments, in vivo administration of the anti-CD2 antibody used in the methods provided herein may exhibit no ADCC activity. In some embodiments, in vivo administration of the anti-CD2 antibody used in the methods provided herein may exhibit at most 5% of the ADCC activity as in vivo administration of siplizumab in a humanized mouse model or a human subject in a clinical setting. In some embodiments, in vivo administration of the anti-CD2 antibody used in the methods provided herein may exhibit at most 10% of the ADCC activity as in vivo administration of siplizumab in a humanized mouse model or a human subject in a clinical setting. In some embodiments, in vivo administration of the anti-CD2 antibody used in the methods provided herein may exhibit at most 20% of the ADCC activity as in vivo administration of siplizumab in a humanized mouse model or a human subject in a clinical setting. In some embodiments, in vivo administration of the anti-CD2 antibody used in the methods provided herein may exhibit at most 30% of the ADCC activity as in vivo administration of siplizumab in a humanized mouse model or a human subject in a clinical setting. In some embodiments, in vivo administration of the anti-CD2 antibody used in the methods provided herein may exhibit at most 40% of the ADCC activity as in vivo administration of siplizumab in a humanized mouse model or a human subject in a clinical setting. In some embodiments, in vivo administration of the anti-CD2 antibody used in the methods provided herein may exhibit at most 50% of the ADCC activity as in vivo administration of siplizumab in a humanized mouse model or a human subject in a clinical setting. In some embodiments, in vivo administration of the anti-CD2 antibody used in the methods provided herein may exhibit at most 60% of the ADCC activity as in vivo administration of siplizumab in a humanized mouse model or a human subject in a clinical setting. In some embodiments, in vivo administration of the anti-CD2 antibody used in the methods provided herein may exhibit at most 70% of the ADCC activity as in vivo administration of siplizumab in a humanized mouse model or a human subject in a clinical setting. In some embodiments, in vivo administration of the anti-CD2 antibody used in the methods provided herein may exhibit at most 80% of the ADCC activity as in vivo administration of siplizumab in a humanized mouse model or a human subject in a clinical setting. In some embodiments, in vivo administration of the anti-CD2 antibody used in the methods provided herein may exhibit at most 90% of the ADCC activity as in vivo administration of siplizumab in a humanized mouse model or a human subject in a clinical setting.

In certain embodiments, an anti-CD2 antibody for use with the present methods and compositions comprises 1, 2, or 3 of the heavy chain CDRs of BTI-322 or of siplizumab. In some embodiments, an anti-CD2 antibody for use with the present methods and compositions comprises one of the heavy chain CDRs of BTI-322. In some embodiments, an anti-CD2 antibody for use with the present methods and compositions comprises one of the heavy chain CDRs of siplizumab. In some embodiments, an anti-CD2 antibody for use with the present methods and compositions comprises two of the heavy chain CDRs of BTI-322. In some embodiments, an anti-CD2 antibody for use with the present methods and compositions comprises two of the heavy chain CDRs of siplizumab. In some embodiments, an anti-CD2 antibody for use with the present methods and compositions comprises three of the heavy chain CDRs of BTI-322. In some embodiments, an anti-CD2 antibody for use with the present methods and compositions comprises three of the heavy chain CDRs of siplizumab.

In certain embodiments, an anti-CD2 antibody for use with the present methods and compositions comprises 1, 2, or 3 of the light chain CDRs of BTI-322 or of siplizumab. In some embodiments, an anti-CD2 antibody for use with the present methods and compositions comprises one of the light chain CDRs of BTI-322. In some embodiments, an anti-CD2 antibody for use with the present methods and compositions comprises one of the light chain CDRs of siplizumab. In some embodiments, an anti-CD2 antibody for use with the present methods and compositions comprises two of the light chain CDRs of BTI-322. In some embodiments, an anti-CD2 antibody for use with the present methods and compositions comprises two of the light chain CDRs of siplizumab. In some embodiments, an anti-CD2 antibody for use with the present methods and compositions comprises three of the light chain CDRs of BTI-322. In some embodiments, an anti-CD2 antibody for use with the present methods and compositions comprises three of the light chain CDRs of siplizumab.

In certain embodiments, an anti-CD2 antibody for use with the present methods and compositions comprises 1, 2, 3, 4, 5, or all 6 of the CDRs of BTI-322 or of siplizumab. In some embodiments, an anti-CD2 antibody for use with the present methods and compositions comprises one of the CDRs of BTI-322. In some embodiments, an anti-CD2 antibody for use with the present methods and compositions comprises two of the CDRs of BTI-322. In some embodiments, an anti-CD2 antibody for use with the present methods and compositions comprises three of the CDRs of BTI-322. In some embodiments, an anti-CD2 antibody for use with the present methods and compositions comprises four of the CDRs of BTI-322. In some embodiments, an anti-CD2 antibody for use with the present methods and compositions comprises five of the CDRs of BTI-322. In some embodiments, an anti-CD2 antibody for use with the present methods and compositions comprises six of the CDRs of BTI-322. In some embodiments, an anti-CD2 antibody for use with the present methods and compositions comprises one of the CDRs of siplizumab. In some embodiments, an anti-CD2 antibody for use with the present methods and compositions comprises two of the CDRs of siplizumab. In some embodiments, an anti-CD2 antibody for use with the present methods and compositions comprises three of the CDRs of siplizumab. In some embodiments, an anti-CD2 antibody for use with the present methods and compositions comprises four of the CDRs of siplizumab. In some embodiments, an anti-CD2 antibody for use with the present methods and compositions comprises five of the CDRs of siplizumab. In some embodiments, an anti-CD2 antibody for use with the present methods and compositions comprises six of the CDRs of siplizumab.

TABLE 1
CDR sequences of siplizumab

| CDR | | |
|---|---|---|
| No. | Heavy Chain | Light Chain |
| 1 | EYYMY | RSSQSLLHSSGNTYLN |
| | (SEQ ID NO: 1) | (SEQ ID NO: 4) |
| 2 | RIDPEDGSIDYVEKFKK | LVSKLES |
| | (SEQ ID NO: 2) | (SEQ ID NO: 5) |
| 3 | GKFNYRFAY | MQFTHYPYT |
| | (SEQ ID NO: 3) | (SEQ ID NO: 6) |

In certain embodiments, the anti-CD2 antibody for use with the methods described herein 1, 2, 3, 4, 5, or all 6 of the CDRs set forth in Table 1. In certain embodiment, 1, 2, 3, 4, 5, or all 6 have 1, 2, 3, 4, 5, 6, 7, 8, 9, or 10 amino acid substitutions. In some embodiments, the CDR set forth in Table 1 has one amino acid substitution. In some embodiments, the CDR set forth in Table 1 has two amino acid substitutions. In some embodiments, the CDR set forth in Table 1 has three amino acid substitutions. In some embodiments, the CDR set forth in Table 1 has four amino acid substitutions. In some embodiments, the CDR set forth in Table 1 has five amino acid substitutions. In some embodiments, the CDR set forth in Table 1 has six amino acid substitutions. In some embodiments, the CDR set forth in Table 1 has seven amino acid substitutions. In some embodiments, the CDR set forth in Table 1 has eight amino acid substitutions. In some embodiments, the CDR set forth in Table 1 has nine amino acid substitutions. In some embodiments, the CDR set forth in Table 1 has ten amino acid substitutions. In a more specific embodiment, such an amino acid substitution is a conservative amino acid substitution.

In certain embodiments, the anti-CD2 antibody for use with the methods provided herein has a heavy chain variable region comprising an amino acid sequence of at least 80%, 85%, 90%, 95%, 98%, at least 99% or 100% identity to SEQ ID NO:7 or SEQ ID NO:9. In some embodiments, the anti-CD2 antibody for use with the methods provided herein has a heavy chain variable region comprising an amino acid sequence of at least 80% identity to SEQ ID NO:7. In some embodiments, the anti-CD2 antibody for use with the methods provided herein has a heavy chain variable region comprising an amino acid sequence of at least 85% identity to SEQ ID NO:7. In some embodiments, the anti-CD2 antibody for use with the methods provided herein has a heavy chain variable region comprising an amino acid sequence of at least 90% identity to SEQ ID NO:7. In some embodiments, the anti-CD2 antibody for use with the methods provided herein has a heavy chain variable region comprising an amino acid sequence of at least 95% identity to SEQ ID NO:7. In some embodiments, the anti-CD2 antibody for use with the methods provided herein has a heavy chain variable region comprising an amino acid sequence of at least 98% identity to SEQ ID NO:7. In some embodiments, the anti-CD2 antibody for use with the methods provided herein has a heavy chain variable region comprising an amino acid sequence of at least 99% identity to SEQ ID NO:7. In some embodiments, the anti-CD2 antibody for use with the methods provided herein has a heavy chain variable region comprising an amino acid sequence of at least 100% identity to SEQ ID NO:7. In some embodiments, the anti-CD2 antibody for use with the methods provided herein has a heavy chain variable region comprising an amino acid sequence of at least 80% identity to SEQ ID NO:9. In some embodiments, the anti-CD2 antibody for use with the methods provided herein has a heavy chain variable region comprising an amino acid sequence of at least 85% identity to SEQ ID NO:9. In some embodiments, the anti-CD2 antibody for use with the methods provided herein has a heavy chain variable region comprising an amino acid sequence of at least 90% identity to SEQ ID NO:9. In some embodiments, the anti-CD2 antibody for use with the methods provided herein has a heavy chain variable region comprising an amino acid sequence of at least 95% identity to SEQ ID NO:9. In some embodiments, the anti-CD2 antibody for use with the methods provided herein has a heavy chain variable region comprising an amino acid sequence of at least 98% identity to SEQ ID NO:9. In some embodiments, the anti-CD2 antibody for use with the methods provided herein has a heavy chain variable region comprising an amino acid sequence of at least 99% identity to SEQ ID NO:9. In some embodiments, the anti-CD2 antibody for use with the methods provided herein has a heavy chain variable region comprising an amino acid sequence of at least 100% identity to SEQ ID NO:9.

In certain embodiments, the anti-CD2 antibody for use with the methods and compositions provided herein has a light chain variable region comprising an amino acid sequence of at least 80%, 85%, 90%, 95%, 98%, at least 99% or 100% identity to SEQ ID NO:8. In some embodiments, the anti-CD2 antibody for use with the methods provided herein has a light chain variable region comprising an amino acid sequence of at least 80% identity to SEQ ID NO:8. In some embodiments, the anti-CD2 antibody for use with the methods provided herein has a light chain variable region comprising an amino acid sequence of at least 85% identity to SEQ ID NO:8. In some embodiments, the anti-CD2 antibody for use with the methods provided herein has a light chain variable region comprising an amino acid sequence of at least 90% identity to SEQ ID NO:8. In some embodiments, the anti-CD2 antibody for use with the methods provided herein has a light chain variable region comprising an amino acid sequence of at least 95% identity to SEQ ID NO:8. In some embodiments, the anti-CD2 antibody for use with the methods provided herein has a light chain variable region comprising an amino acid sequence of at least 98% identity to SEQ ID NO:8. In some embodiments, the anti-CD2 antibody for use with the methods provided herein has a light chain variable region comprising an amino acid sequence of at least 99% identity to SEQ ID NO:8. In some embodiments, the anti-CD2 antibody for use with the methods provided herein has a light chain variable region comprising an amino acid sequence of at least 100% identity to SEQ ID NO:8.

In certain embodiments, the anti-CD2 antibody for use with the methods provided herein has a heavy chain variable region comprising VH CDRs of SEQ ID NO Nos 1-3, respectively, and a VL of SEQ ID NO: 8. In certain embodiments, the anti-CD2 antibody for use with the methods provided herein has a heavy chain variable region comprising VL CDRs of SEQ ID NOs: 4-6, respectively, and a VH of SEQ ID NO: 7.

In certain embodiments, the anti-CD2 antibody binds specifically to the same epitope in human CD2 as siplizumab.

In certain embodiments, the anti-CD2 antibody can be an animal-specific antibody, a human-specific antibody, a chimeric antibody, a humanized antibody, a be full length antibody, an antibody fragment, a single chain variable fragment (scFv), a naturally occurring antibody, a synthetic antibody, an engineered antibody, or a combination of these. In a specific embodiment, the anti-CD2 antibody can be a humanized anti-CD2 monoclonal antibody.

In certain embodiments, a method of treatment provided herein comprises administering an anti-CD2 antibody to a transplant recipient prior to transplant. In some embodiments, an anti-CD2 antibody can be administered to the recipient 1 day prior to transplant, 2 days prior to transplant, 3 days prior to transplant, 4 days prior to transplant, 1 day and 2 days prior to transplant, 2 days and 3 days prior to transplant, 3 days and 4 days prior to transplant, 1 day prior and 2 days and 3 days prior to transplant, or 1 day prior and 2 days prior and 3 days prior and 4 days prior to transplant. In some embodiments, an anti-CD2 antibody is administered to the recipient 1 day prior to the transplant. In some embodiments, an anti-CD2 antibody is administered to the recipient 2 days prior to the transplant. In some embodiments, an anti-CD2 antibody is administered to the recipient 3 days prior to the transplant. In some embodiments, an anti-CD2 antibody is administered to the recipient 4 days prior to the transplant. In a specific embodiment, the anti-CD2 antibody can be administered to a recipient 1 day prior and 2 days prior to transplant. In some embodiments, the anti-CD2 antibody can be administered to a recipient 2 days prior and 3 days prior to transplant. In some embodiments, the anti-CD2 antibody can be administered to a recipient 3 days prior and 4 days prior to transplant. In some embodiments, the anti-CD2 antibody can be administered to a recipient 1 day prior and 2 days and 3 days prior to transplant. In some embodiments, the anti-CD2 antibody can be administered to a recipient 1 day prior and 2 days prior and 3 days prior and 4 days prior to transplant.

In particular embodiments, an anti-CD2 antibody is administered on the day of the transplant surgery.

In certain embodiments, a method of treatment provided herein comprises administering an anti-CD2 antibody to a transplant recipient after the transplant. In some embodiments, an anti-CD2 antibody can be administered to the recipient 1 day after transplant, 2 days after transplant, 3 days after transplant, 4 days after transplant, 5 days after transplant, 6 days after transplant, 7 days after transplant, 8 days after transplant, 9 days after transplant, 10 days after transplant, 11 days after transplant, 12 days after transplant, 13 days after transplant, 14 days after transplant, 15 days after transplant, 16 days after transplant, 17 days after transplant, 18 days after transplant, 19 days after transplant, 20 days after transplant, 21 days after transplant, 22 days after transplant, 23 days after transplant, 24 days after transplant, 25 days after transplant, 26 days after transplant, 27 days after transplant, 28 days after transplant, 29 days after transplant, and/or 30 days after transplant. In specific embodiments, an anti-CD2 antibody is administered on the day of transplant, 1 day after transplant, 2 days after transplant and/or 4 days after transplant. In some embodiments, an anti-CD2 antibody is administered on the day of transplant. In some embodiments, an anti-CD2 antibody is administered to the recipient 1 day after transplant. In some embodiments, an anti-CD2 antibody is administered to the recipient 2 days after transplant. In some embodiments, an anti-CD2 antibody is administered to the recipient 3 days after transplant. In some embodiments, an anti-CD2 antibody is administered to the recipient 4 days after transplant. In some embodiments, an anti-CD2 antibody is administered to the recipient 5 days after transplant. In some embodiments, an anti-CD2 antibody is administered to the recipient 6 days after transplant. In some embodiments, an anti-CD2 antibody is administered to the recipient 7 days after transplant. In some embodiments, an anti-CD2 antibody is administered to the recipient 8 days after transplant. In some embodiments, an anti-CD2 antibody is administered to the recipient 9 days after transplant. In some embodiments, an anti-CD2 antibody is administered to the recipient 10 days after transplant. In some embodiments, an anti-CD2 antibody is administered to the recipient 11 days after transplant. In some embodiments, an anti-CD2 antibody is administered to the recipient 12 days after transplant. In some embodiments, an anti-CD2 antibody is administered to the recipient 14 days after transplant. In some embodiments, an anti-CD2 antibody is administered to the recipient 15 days after transplant. In some embodiments, an anti-CD2 antibody is administered to the recipient 16 days after transplant. In some embodiments, an anti-CD2 antibody is administered to the recipient 17 days after transplant. In some embodiments, an anti-CD2 antibody is administered to the recipient 18 days after transplant. In some embodiments, an anti-CD2 antibody is administered to the recipient 19 days after transplant. In some embodiments, an anti-CD2 antibody is administered to the recipient 20 days after transplant. In some embodiments, an anti-CD2 antibody is administered to the recipient 21 days after transplant. In some embodiments, an anti-CD2 antibody is administered to the recipient 22 days after transplant. In some embodiments, an anti-CD2 antibody is administered to the recipient 23 days after transplant. In some embodiments, an anti-CD2 antibody is administered to the recipient 24 days after transplant. In some embodiments, an anti-CD2 antibody is administered to the recipient 25 days after transplant. In some embodiments, an anti-CD2 antibody is administered to the recipient 26 days after transplant. In some embodiments, an anti-CD2 antibody is administered to the recipient 27 days after transplant. In some embodiments, an anti-CD2 antibody is administered to the recipient 28 days after transplant. In some embodiments, an anti-CD2 antibody is administered to the recipient 29 days after transplant. In some embodiments, an anti-CD2 antibody is administered to the recipient 30 days after transplant.

In certain embodiments, a test dose of the anti-CD2 antibody can be administered. In certain embodiments, the administration of the test dose is optional.

In certain embodiments, the anti-CD2 antibody can be administered to a transplant recipient at a dose amount of 0.05 mg/kg/dose, 0.1 mg/kg/dose, 0.15 mg/kg/dose, 0.2 mg/kg/dose, 0.25 mg/kg/dose, 0.3 mg/kg/dose, 0.35 mg/kg/dose, 0.4 mg/kg/dose, 0.45 mg/kg/dose, 0.5 mg/kg/dose, 0.55 mg/kg/dose, 0.6 mg/kg/dose, 0.65 mg/kg/dose, 0.7 mg/kg/dose, 0.75 mg/kg/dose, 0.8 mg/kg/dose, 0.85 mg/kg/dose, 0.9 mg/kg/dose, 0.95 mg/kg/dose, 1.0 mg/kg/dose, 1.5 mg/kg/dose, 2 mg/kg/dose, 2.5 mg/kg/dose, 3 mg/kg/dose, 3.5 mg/kg/dose, 4 mg/kg/dose, 4.5 mg/kg/dose, 5 mg/kg/dose, 5.5 mg/kg/dose, 6 mg/kg/dose, 6.5 mg/kg/dose, 7 mg/kg/dose, 7.5 mg/kg/dose, 8 mg/kg/dose, 8.5 mg/kg/dose, 9 mg/kg/dose, 9.5 mg/kg/dose, or 10 mg/kg/dose. In some embodiments, the anti-CD2 antibody is administered to a transplant recipient at a dose of 0.05 mg/kg/dose. In some embodiments, the anti-CD2 antibody is administered to a transplant recipient at a dose of 0.1 mg/kg/dose. In some embodiments, the anti-CD2 antibody is administered to a transplant recipient at a dose of 0.15 mg/kg/dose. In some embodiments, the anti-CD2 antibody is administered to a transplant recipient at a dose of 0.2 mg/kg/dose. In some embodiments, the anti-CD2 antibody is administered to a transplant recipient at a dose of 0.25 mg/kg/dose. In some embodiments, the anti-CD2 antibody is administered to a transplant recipient at a dose of 0.3 mg/kg/dose. In some embodiments, the anti-CD2 antibody is administered to a transplant recipient at a dose of 0.35 mg/kg/dose. In some embodiments, the anti-CD2 antibody is administered to a transplant recipient at a dose of 0.4 mg/kg/dose. In some embodiments, the anti-CD2 antibody is administered to a transplant recipient at a dose of 0.45 mg/kg/dose. In some embodiments, the anti-CD2 antibody is administered to a transplant recipient at a dose of 0.5 mg/kg/dose. In some embodiments, the anti-CD2 antibody is administered to a transplant recipient at a dose of 0.55 mg/kg/dose. In some embodiments, the anti-CD2 antibody is administered to a transplant recipient at a dose of 0.6 mg/kg/dose. In some embodiments, the anti-CD2 antibody is administered to a transplant recipient at a dose of 0.65 mg/kg/dose. In some embodiments, the anti-CD2 antibody is administered to a transplant recipient at a dose of 0.7 mg/kg/dose. In some embodiments, the anti-CD2 antibody is administered to a transplant recipient at a dose of 0.75 mg/kg/dose. In some embodiments, the anti-CD2 antibody is administered to a transplant recipient at a dose of 0.8 mg/kg/dose. In some embodiments, the anti-CD2 antibody is administered to a transplant recipient at a dose of 0.85 mg/kg/dose. In some embodiments, the anti-CD2 antibody is administered to a transplant recipient at a dose of 0.9 mg/kg/dose. In some embodiments, the anti-CD2 antibody is administered to a transplant recipient at a dose of 0.95 mg/kg/dose. In some embodiments, the anti-CD2 antibody is administered to a transplant recipient at a dose of 1.0 mg/kg/dose. In some embodiments, the anti-CD2 antibody is administered to a transplant recipient at a dose of 1.5 mg/kg/dose. In some embodiments, the anti-CD2 antibody is administered to a transplant recipient at a dose of 2 mg/kg/dose. In some embodiments, the anti-CD2 antibody is administered to a transplant recipient at a dose of 2.5 mg/kg/dose. In some embodiments, the anti-CD2 antibody is administered to a transplant recipient at a dose of 3 mg/kg/dose. In some embodiments, the anti-CD2 antibody is administered to a transplant recipient at a dose of 3.5 mg/kg/dose. In some embodiments, the anti-CD2 antibody is administered to a transplant recipient at a dose of 4 mg/kg/dose. In some embodiments, the anti-CD2 antibody is administered to a transplant recipient at a dose of 4.5 mg/kg/dose. In some embodiments, the anti-CD2 antibody is administered to a transplant recipient at a dose of 5 mg/kg/dose. In some embodiments, the anti-CD2 antibody is administered to a transplant recipient at a dose of 5.5 mg/kg/dose. In some embodiments, the anti-CD2 antibody is administered to a transplant recipient at a dose of 6 mg/kg/dose. In some embodiments, the anti-CD2 antibody is administered to a transplant recipient at a dose of 6.5 mg/kg/dose. In some embodiments, the anti-CD2 antibody is administered to a transplant recipient at a dose of 7 mg/kg/dose. In some embodiments, the anti-CD2 antibody is administered to a transplant recipient at a dose of 7.5 mg/kg/dose. In some embodiments, the anti-CD2 antibody is administered to a transplant recipient at a dose of 8 mg/kg/dose. In some embodiments, the anti-CD2 antibody is administered to a transplant recipient at a dose of 8.5 mg/kg/dose. In some embodiments, the anti-CD2 antibody is administered to a transplant recipient at a dose of 9 mg/kg/dose. In some embodiments, the anti-CD2 antibody is administered to a transplant recipient at a dose of 9.5 mg/kg/dose. In some embodiments, the anti-CD2 antibody is administered to a transplant recipient at a dose of 10 mg/kg/dose.

In certain embodiments, the anti-CD2 antibody can be administered to a transplant recipient at dose ranges of 0.1-0.3 mg/kg/dose, 0.2-0.4 mg/kg/dose, 0.3-0.5 mg/kg/dose, 0.4-0.6 mg/kg/dose, 0.45-0.65 mg/kg/dose, 0.5-0.7 mg/kg, 0.55-0.75 mg/kg/dose, 0.6-0.8 mg/kg/dose, 0.65-0.85 mg/kg/dose, 0.7-0.9 mg/kg/dose, 0.8-1.0 mg/kg/dose, 1-1.5 mg/kg/dose, 1.5-2 mg/kg/dose, 2-2.5 mg/kg/dose, 2.5-3 mg/kg/dose, 3-3.5 mg/kg/dose, 3.5-4 mg/kg/dose, 4-4.5 mg/kg/dose, or 4.5-5 mg/kg/dose. In a specific embodiment, the anti-CD2 antibody can be administered to a transplant recipient at a dose amount of 0.6 mg/kg/dose. In some embodiments, the anti-CD2 antibody is administered to a transplant recipient at a dose of 0.1-5 mg/kg/dose. In some embodiments, the anti-CD2 antibody is administered to a transplant recipient at a dose of 0.2-4.5 mg/kg/dose. In some embodiments, the anti-CD2 antibody is administered to a transplant recipient at a dose of 0.3-4 mg/kg/dose. In some embodiments, the anti-CD2 antibody is administered to a transplant recipient at a dose of 0.4-3.5 mg/kg/dose. In some embodiments, the anti-CD2 antibody is administered to a transplant recipient at a dose of 0.1-1.0 mg/kg/dose. In some embodiments, the anti-CD2 antibody is administered to a transplant recipient at a dose of 0.2-0.9 mg/kg/dose. In some embodiments, the anti-CD2 antibody is administered to a transplant recipient at a dose of 0.3-0.8 mg/kg/dose. In some embodiments, the anti-CD2 antibody is administered to a transplant recipient at a dose of 0.4-0.7 mg/kg/dose. In some embodiments, the anti-CD2 antibody is administered to a transplant recipient at a dose of 0.1-0.3 mg/kg/dose. In some embodiments, the anti-CD2 antibody is administered to a transplant recipient at a dose of 0.2-0.4 mg/kg/dose. In some embodiments, the anti-CD2 antibody is administered to a transplant recipient at a dose of 0.3-0.5 mg/kg/dose. In some embodiments, the anti-CD2 antibody is administered to a transplant recipient at a dose of 0.4-0.6 mg/kg/dose. In some embodiments, the anti-CD2 antibody is administered to a transplant recipient at a dose of 0.45-0.65 mg/kg/dose. In some embodiments, the anti-CD2 antibody is administered to a transplant recipient at a dose of 0.5-0.7 mg/kg. In some embodiments, the anti-CD2 antibody is administered to a transplant recipient at a dose of 0.55-0.75 mg/kg/dose. In some embodiments, the anti-CD2 antibody is administered to a transplant recipient at a dose of 0.6-0.8 mg/kg/dose. In some embodiments, the anti-CD2 antibody is administered to a transplant recipient at a dose of 0.65-0.85 mg/kg/dose. In some embodiments, the anti-CD2 antibody is administered to a transplant recipient at a dose of 0.7-0.9 mg/kg/dose. In some embodiments, the anti-CD2 antibody is administered to a transplant recipient at a dose of 0.8-1.0 mg/kg/dose. In some embodiments, the anti-CD2 antibody is administered to a transplant recipient at a dose of 1.0-1.5 mg/kg/dose. In some embodiments, the anti-CD2 antibody is administered to a transplant recipient at a dose of 1.5-2 mg/kg/dose. In some embodiments, the anti-CD2 antibody is administered to a transplant recipient at a dose of 2-2.5 mg/kg/dose. In some embodiments, the anti-CD2 antibody is administered to a transplant recipient at a dose of 2.5-3 mg/kg/dose. In some embodiments, the anti-CD2 antibody is administered to a transplant recipient at a dose of 3-3.5 mg/kg/dose. In some embodiments, the anti-CD2 antibody is administered to a transplant recipient at a dose of 3.5-4 mg/kg/dose. In some embodiments, the anti-CD2 antibody is administered to a transplant recipient at a dose of 4-4.5 mg/kg/dose. In some embodiments, the anti-CD2 antibody is administered to a transplant recipient at a dose of 4.5-5 mg/kg/dose.

In certain embodiments, the anti-CD2 antibody can be administered to a transplant recipient in a convenient manner known in the art including subcutaneously, intravenously, intravascularly, topically, intra-arterially, intra-cranially, intramuscularly, orally, intra-orbitally, by inhalation, transdermally, intra-peritoneally, or through a route of administration which allows for the depletion of T-cells in the recipient. In a specific embodiment, the anti-CD2 antibody is administered intravenously. In some embodiments, the anti-CD2 antibody is administered subcutaneously. In some embodiments, the anti-CD2 antibody is administered intravascularly. In some embodiments, the anti-CD2 antibody is administered topically. In some embodiments, the anti-CD2 antibody is administered intra-arterially. In some embodiments, the anti-CD2 antibody is administered intra-cranially. In some embodiments, the anti-CD2 antibody is administered intramuscularly. In some embodiments, the anti-CD2 antibody is administered orally. In some embodiments, the anti-CD2 antibody is administered intra-orbitally. In some embodiments, the anti-CD2 antibody is administered by inhalation. In some embodiments, the anti-CD2 antibody is administered transdermally. In some embodiments, the anti-CD2 antibody is administered intra-peritoneally. In some embodiments, the anti-CD2 antibody is administered through a route of administration which allows for the depletion of T-cells in the recipient.

In certain embodiments, the anti-CD2 antibody is a humanized monoclonal antibody. In a specific embodiment, the anti-CD2 antibody can be Siplizumab (MEDI-507). In certain embodiments, the administration of the anti-CD2 antibody can be modified as described herein. In some embodiments, methylprednisolone is given prior to the administration of the anti-CD2 antibody. In some embodiments, acetaminophen (e.g., 1000 mg) and/or an antihistamine (e.g., 25 mg of an H1-antagonist such as diphenhydramine) is administered prior to the administration of the anti-CD2 antibody. In some embodiments, acetaminophen (e.g., 1000 mg) is administered prior to the administration of the anti-CD2 antibody. In some embodiments, an antihistamine (e.g., 25 mg of an H1-antagonist such as diphenhydramine) is administered prior to the administration of the anti-CD2 antibody. In some embodiments, acetaminophen (e.g., 1000 mg) and an antihistamine (e.g., 25 mg of an H1-antagonist such as diphenhydramine) are administered prior to the administration of the anti-CD2 antibody.

Without being bound by theory, the anti-CD2 antibody increases the level of regulatory T cells in the recipient. Specifically, the anti-CD2 antibody can increase the level of FOXP3⁺ regulatory T cells in the recipient, e.g., by at least 10%, 20%, 30%, 40%, 50%, 60%, 70%, 80%, 90%, 100%, 125%, 150%, 175%, 200%, or by at least 250% relative to FOXP3+ regulatory T cells without treatment with the anti-CD2 antibody. In some embodiments, the anti-CD2 antibody can increase the level of FOXP3⁺ regulatory T cells in the recipient by at least 10%, relative to FOXP3⁺ regulatory T cells without treatment with the anti-CD2 antibody. In some embodiments, the anti-CD2 antibody can increase the level of FOXP3⁺ regulatory T cells in the recipient by at least 20%, relative to FOXP3⁺ regulatory T cells without treatment with the anti-CD2 antibody. In some embodiments, the anti-CD2 antibody can increase the level of FOXP3⁺ regulatory T cells in the recipient by at least 30%, relative to FOXP3⁺ regulatory T cells without treatment with the anti-CD2 antibody. In some embodiments, the anti-CD2 antibody can increase the level of FOXP3⁺ regulatory T cells in the recipient by at least 40%, relative to FOXP3⁺ regulatory T cells without treatment with the anti-CD2 antibody. In some embodiments, the anti-CD2 antibody can increase the level of FOXP3⁺ regulatory T cells in the recipient by at least 50%, relative to FOXP3⁺ regulatory T cells without treatment with the anti-CD2 antibody. In some embodiments, the anti-CD2 antibody can increase the level of FOXP3⁺ regulatory T cells in the recipient by at least 60%, relative to FOXP3⁺ regulatory T cells without treatment with the anti-CD2 antibody. In some embodiments, the anti-CD2 antibody can increase the level of FOXP3⁺ regulatory T cells in the recipient by at least 70%, relative to FOXP3⁺ regulatory T cells without treatment with the anti-CD2 antibody. In some embodiments, the anti-CD2 antibody can increase the level of FOXP3⁺ regulatory T cells in the recipient by at least 80%, relative to FOXP3⁺ regulatory T cells without treatment with the anti-CD2 antibody. In some embodiments, the anti-CD2 antibody can increase the level of FOXP3Y regulatory T cells in the recipient by at least 90%, relative to FOXP3⁺ regulatory T cells without treatment with the anti-CD2 antibody. In some embodiments, the anti-CD2 antibody can increase the level of FOXP3+ regulatory T cells in the recipient by at least 100%, relative to FOXP3⁺ regulatory T cells without treatment with the anti-CD2 antibody. In some embodiments, the anti-CD2 antibody can increase the level of FOXP3⁺ regulatory T cells in the recipient by at least 125%, relative to FOXP3⁺ regulatory T cells without treatment with the anti-CD2 antibody. In some embodiments, the anti-CD2 antibody can increase the level of FOXP3⁺ regulatory T cells in the recipient by at least 150%, relative to FOXP3⁺ regulatory T cells without treatment with the anti-CD2 antibody. In some embodiments, the anti-CD2 antibody can increase the level of FOXP3⁺ regulatory T cells in the recipient by at least 175%, relative to FOXP3⁺ regulatory T cells without treatment with the anti-CD2 antibody. In some embodiments, the anti-CD2 antibody can increase the level of FOXP3⁺ regulatory T cells in the recipient by at least 200%, relative to FOXP3⁺ regulatory T cells without treatment with the anti-CD2 antibody. In some embodiments, the anti-CD2 antibody can increase the level of FOXP3⁺ regulatory T cells in the recipient by at least 250%, relative to FOXP3⁺ regulatory T cells without treatment with the anti-CD2 antibody.

### 7.3.2 Cyclophosphamide

Cyclophosphamide, as used herein, is a compound administered to the recipient to suppress the immune system. Cyclophosphamide (2-[bis(2-chloroethyl)amino]tetrahydro-2H-1,3,2-oxazaphosphorine 2-oxide monohydrate) is administered to induce tolerance towards the transplanted organ (e.g., liver) and brand names of cyclophosphamide include Cytoxan®, Neosar®, and Endoxan®.

In certain embodiments, the conditioning regimen provided herein comprises administering cyclophosphamide to a transplant recipient prior to transplant. In some embodiments, the cyclophosphamide is administered to the recipient at least once during the 30 days after the transplant. In some embodiments, the cyclophosphamide can be administered to the recipient three days after transplant, four days after transplant, five days after transplant, six days after transplant, three days and four days after transplant, five days and six days after transplant, three days and four days and five days after transplant, or four days and five days and six days after transplant. In a specific embodiment, cyclophosphamide can be administered to a recipient four days and five days after transplant. In specific embodiments, the cyclophosphamide is first administered to a recipient three days, four days, five days, six days, or seven days after the transplant. In some embodiments, the cyclophosphamide can be administered to the recipient three days after transplant. In some embodiments, the cyclophosphamide can be administered to the recipient four days after transplant. In some embodiments, the cyclophosphamide can be administered to the recipient five days after transplant. In some embodiments, the cyclophosphamide can be administered to the recipient six days after transplant. In some embodiments, the cyclophosphamide can be administered to the recipient three days and four days after transplant. In some embodiments, the cyclophosphamide can be administered to the recipient five days and six days after transplant. In some embodiments, the cyclophosphamide can be administered to the recipient three days and four days and five days after transplant. In some embodiments, the cyclophosphamide can be administered to the recipient four days and five days and six days after transplant. In a specific embodiment, cyclophosphamide can be administered to a recipient four days and five days after transplant. In specific embodiments, the cyclophosphamide is first administered to a recipient three days, four days, five days, six days, or seven days after the transplant.

In particular embodiments, a recipient treated in accordance with a method described herein has a MELD score of 30 or higher and the method comprises first administering cyclophosphamide to the recipient 1-2 days after the transplant, 2-4 days after the transplant, 4-6 days after the transplant, 6-8 days after the transplant, 8-10 days after the transplant, 10-12 days after the transplant, 12-14 days after the transplant, 14-16 days after the transplant, 16-18 days after the transplant, 18-20 days after the transplant, 20-22 days after the transplant, 22-24 days after the transplant, 24-26 days after the transplant, 26-28 days after the transplant, 28-30 days after the transplant or more than 30 days after the transplant. In some embodiments, a recipient treated in accordance with a method described herein has a MELD score of 30 or higher and the method comprises first administering cyclophosphamide to the recipient 1-2 days after the transplant. In some embodiments, a recipient treated in accordance with a method described herein has a MELD score of 30 or higher and the method comprises first administering cyclophosphamide to the recipient 2-4 days after the transplant. In some embodiments, a recipient treated in accordance with a method described herein has a MELD score of 30 or higher and the method comprises first administering cyclophosphamide to the recipient 4-6 days after the transplant. In some embodiments, a recipient treated in accordance with a method described herein has a MELD score of 30 or higher and the method comprises first administering cyclophosphamide to the recipient 6-8 days after the transplant. In some embodiments, a recipient treated in accordance with a method described herein has a MELD score of 30 or higher and the method comprises first administering cyclophosphamide to the recipient 8-10 days after the transplant. In some embodiments, a recipient treated in accordance with a method described herein has a MELD score of 30 or higher and the method comprises first administering cyclophosphamide to the recipient 10-12 days after the transplant. In some embodiments, a recipient treated in accordance with a method described herein has a MELD score of 30 or higher and the method comprises first administering cyclophosphamide to the recipient 12-14 days after the transplant. In some embodiments, a recipient treated in accordance with a method described herein has a MELD score of 30 or higher and the method comprises first administering cyclophosphamide to the recipient 14-16 days after the transplant. In some embodiments, a recipient treated in accordance with a method described herein has a MELD score of 30 or higher and the method comprises first administering cyclophosphamide to the recipient 16-18 days after the transplant. In some embodiments, a recipient treated in accordance with a method described herein has a MELD score of 30 or higher and the method comprises first administering cyclophosphamide to the recipient 18-20 days after the transplant. In some embodiments, a recipient treated in accordance with a method described herein has a MELD score of 30 or higher and the method comprises first administering cyclophosphamide to the recipient 20-22 days after the transplant. In some embodiments, a recipient treated in accordance with a method described herein has a MELD score of 30 or higher and the method comprises first administering cyclophosphamide to the recipient 22-24 days after the transplant. In some embodiments, a recipient treated in accordance with a method described herein has a MELD score of 30 or higher and the method comprises first administering cyclophosphamide to the recipient 24-26 days after the transplant. In some embodiments, a recipient treated in accordance with a method described herein has a MELD score of 30 or higher and the method comprises first administering cyclophosphamide to the recipient 26-28 days after the transplant. In some embodiments, a recipient treated in accordance with a method described herein has a MELD score of 30 or higher and the method comprises first administering cyclophosphamide to the recipient 28-30 days after the transplant. In some embodiments, a recipient treated in accordance with a method described herein has a MELD score of 30 or higher and the method comprises first administering cyclophosphamide to the recipient more than 30 days after the transplant.

In particular embodiments, a recipient treated in accordance with a method described herein has a MELD score of 30 or less and the method comprises first administering cyclophosphamide to the recipient 1 day after the transplant, 2 days after the transplant, 3 days after the transplant, 4 days after the transplant, 5 days after the transplant, 6 days after the transplant, 7 days after the transplant, 8 days after the transplant, 9 days after the transplant, 10 days after the transplant, 11 days after the transplant, 12 days after the transplant, 13 days after the transplant, 14 days after the transplant, or more than 14 days after the transplant. In some embodiments, a recipient treated in accordance with a method described herein has a MELD score of 30 or less and the method comprises first administering cyclophosphamide to the recipient 1 day after the transplant. In some embodiments, a recipient treated in accordance with a method described herein has a MELD score of 30 or less and the method comprises first administering cyclophosphamide to the recipient 2 days after the transplant. In some embodiments, a recipient treated in accordance with a method described herein has a MELD score of 30 or less and the method comprises first administering cyclophosphamide to the recipient 3 days after the transplant. In some embodiments, a recipient treated in accordance with a method described herein has a MELD score of 30 or less and the method comprises first administering cyclophosphamide to the recipient 4 days after the transplant. In some embodiments, a recipient treated in accordance with a method described herein has a MELD score of 30 or less and the method comprises first administering cyclophosphamide to the recipient 5 days after the transplant. In some embodiments, a recipient treated in accordance with a method described herein has a MELD score of 30 or less and the method comprises first administering cyclophosphamide to the recipient 6 days after the transplant. In some embodiments, a recipient treated in accordance with a method described herein has a MELD score of 30 or less and the method comprises first administering cyclophosphamide to the recipient 7 days after the transplant. In some embodiments, a recipient treated in accordance with a method described herein has a MELD score of 30 or less and the method comprises first administering cyclophosphamide to the recipient 8 days after the transplant. In some embodiments, a recipient treated in accordance with a method described herein has a MELD score of 30 or less and the method comprises first administering cyclophosphamide to the recipient 9 days after the transplant. In some embodiments, a recipient treated in accordance with a method described herein has a MELD score of 30 or less and the method comprises first administering cyclophosphamide to the recipient 10 days after the transplant. In some embodiments, a recipient treated in accordance with a method described herein has a MELD score of 30 or less and the method comprises first administering cyclophosphamide to the recipient 11 days after the transplant. In some embodiments, a recipient treated in accordance with a method described herein has a MELD score of 30 or less and the method comprises first administering cyclophosphamide to the recipient 12 days after the transplant. In some embodiments, a recipient treated in accordance with a method described herein has a MELD score of 30 or less and the method comprises first administering cyclophosphamide to the recipient 13 days after the transplant. In some embodiments, a recipient treated in accordance with a method described herein has a MELD score of 30 or less and the method comprises first administering cyclophosphamide to the recipient 14 days after the transplant. In some embodiments, a recipient treated in accordance with a method described herein has a MELD score of 30 or less and the method comprises first administering cyclophosphamide to the recipient more than 14 days after the transplant.

In certain embodiments, the cyclophosphamide can be administered to a transplant recipient at a dose amount of 20 mg/kg/dose, 25 mg/kg/dose, 30 mg/kg/dose, 35 mg/kg/dose, 40 mg/kg/dose, 45 mg/kg/dose, 50 mg/kg/dose, 55 mg/kg/dose, 56 mg/kg/dose, 57 mg/kg/dose, 58 mg/kg/dose, 59 mg/kg/dose, 60 mg/kg/dose, 61 mg/kg/dose, 62 mg/kg/dose, 63 mg/kg/dose, 64 mg/kg/dose, 65 mg/kg/dose, 70 mg/kg/dose, 75 mg/kg/dose, 80 mg/kg/dose, 85 mg/kg/dose, or 90 mg/kg/dose. In some embodiments, the cyclophosphamide is administered to a transplant recipient at a dose of 20 mg/kg/dose. In some embodiments, the cyclophosphamide is administered to a transplant recipient at a dose of 25 mg/kg/dose. In some embodiments, the cyclophosphamide is administered to a transplant recipient at a dose of 30 mg/kg/dose. In some embodiments, the cyclophosphamide is administered to a transplant recipient at a dose of 35 mg/kg/dose. In some embodiments, the cyclophosphamide is administered to a transplant recipient at a dose of 40 mg/kg/dose. In some embodiments, the cyclophosphamide is administered to a transplant recipient at a dose of 45 mg/kg/dose. In some embodiments, the cyclophosphamide is administered to a transplant recipient at a dose of 50 mg/kg/dose. In some embodiments, the cyclophosphamide is administered to a transplant recipient at a dose of 55 mg/kg/dose. In some embodiments, the cyclophosphamide is administered to a transplant recipient at a dose of 56 mg/kg/dose. In some embodiments, the cyclophosphamide is administered to a transplant recipient at a dose of 57 mg/kg/dose. In some embodiments, the cyclophosphamide is administered to a transplant recipient at a dose of 58 mg/kg/dose. In some embodiments, the cyclophosphamide is administered to a transplant recipient at a dose of 59 mg/kg/dose. In some embodiments, the cyclophosphamide is administered to a transplant recipient at a dose of 60 mg/kg/dose. In some embodiments, the cyclophosphamide is administered to a transplant recipient at a dose of 61 mg/kg/dose. In some embodiments, the cyclophosphamide is administered to a transplant recipient at a dose of 62 mg/kg/dose. In some embodiments, the cyclophosphamide is administered to a transplant recipient at a dose of 63 mg/kg/dose. In some embodiments, the cyclophosphamide is administered to a transplant recipient at a dose of 64 mg/kg/dose. In some embodiments, the cyclophosphamide is administered to a transplant recipient at a dose of 65 mg/kg/dose. In some embodiments, the cyclophosphamide is administered to a transplant recipient at a dose of 70 mg/kg/dose. In some embodiments, the cyclophosphamide is administered to a transplant recipient at a dose of 75 mg/kg/dose. In some embodiments, the cyclophosphamide is administered to a transplant recipient at a dose of 80 mg/kg/dose. In some embodiments, the cyclophosphamide is administered to a transplant recipient at a dose of 85 mg/kg/dose. In some embodiments, the cyclophosphamide is administered to a transplant recipient at a dose of 90 mg/kg/dose.

In certain embodiments, the cyclophosphamide can be administered to a transplant recipient at dose ranges of 20-30 mg/kg/dose, 25-35 mg/kg/dose, 30-40 mg/kg/dose, 35-45 mg/kg/dose, 40-50 mg/kg/dose, 45-55 mg/kg/dose, 50-60 mg/kg, 55-65 mg/kg/dose, 60-70 mg/kg/dose, 65-75 mg/kg/dose, 70-80 mg/kg/dose, 75-85 mg/kg/dose, or 80-90 mg/kg/dose. In a specific embodiment, the cyclophosphamide can be administered to a transplant recipient at a dose amount of 40 mg/kg/dose. In some embodiments, the cyclophosphamide is administered to a transplant recipient at a dose range of 20-100 mg/kg/dose. In some embodiments, the cyclophosphamide is administered to a transplant recipient at a dose range of 25-90 mg/kg/dose. In some embodiments, the cyclophosphamide is administered to a transplant recipient at a dose range of 30-70 mg/kg/dose. In some embodiments, the cyclophosphamide is administered to a transplant recipient at a dose range of 35-60 mg/kg/dose. In some embodiments, the cyclophosphamide is administered to a transplant recipient at a dose range of 40-50 mg/kg/dose. In some embodiments, the cyclophosphamide is administered to a transplant recipient at a dose range of 20-30 mg/kg/dose. In some embodiments, the cyclophosphamide is administered to a transplant recipient at a dose range of 25-35 mg/kg/dose. In some embodiments, the cyclophosphamide is administered to a transplant recipient at a dose range of 30-40 mg/kg/dose. In some embodiments, the cyclophosphamide is administered to a transplant recipient at a dose range of 35-45 mg/kg/dose. In some embodiments, the cyclophosphamide is administered to a transplant recipient at a dose range of 40-50 mg/kg/dose. In some embodiments, the cyclophosphamide is administered to a transplant recipient at a dose range of 45-55 mg/kg/dose. In some embodiments, the cyclophosphamide is administered to a transplant recipient at a dose range of 50-60 mg/kg. In some embodiments, the cyclophosphamide is administered to a transplant recipient at a dose range of 55-65 mg/kg/dose. In some embodiments, the cyclophosphamide is administered to a transplant recipient at a dose range of 60-70 mg/kg/dose. In some embodiments, the cyclophosphamide is administered to a transplant recipient at a dose range of 65-75 mg/kg/dose. In some embodiments, the cyclophosphamide is administered to a transplant recipient at a dose range of 70-80 mg/kg/dose. In some embodiments, the cyclophosphamide is administered to a transplant recipient at a dose range of 75-85 mg/kg/dose. In some embodiments, the cyclophosphamide is administered to a transplant recipient at a dose range of 80-90 mg/kg/dose. In a specific embodiment, the cyclophosphamide can be administered to a transplant recipient at a dose amount of 40 mg/kg/dose.

In certain embodiments, the cyclophosphamide can be administered to a transplant recipient in a convenient manner known in the art including subcutaneously, intravenously, intravascularly, topically, intraarterially, intracranially, intramuscularly, orally, intraorbitally, by inhalation, transdermally, intraperitoneally, or through a route of administration which allows for the proper action of the cyclophosphamide by the recipient. In a specific embodiment, the cyclophosphamide is administration intravenously. In particular embodiments, the cyclophosphamide is administered intravenously over a one hour. In some embodiments, the cyclophosphamide is administered subcutaneously. In some embodiments, the cyclophosphamide is administered intravascularly. In some embodiments, the cyclophosphamide is administered topically. In some embodiments, the cyclophosphamide is administered intra-arterially. In some embodiments, the cyclophosphamide is administered intra-cranially. In some embodiments, the cyclophosphamide is administered intramuscularly. In some embodiments, the cyclophosphamide is administered orally. In some embodiments, the cyclophosphamide is administered intra-orbitally. In some embodiments, the cyclophosphamide is administered by inhalation. In some embodiments, the cyclophosphamide is administered transdermally. In some embodiments, the cyclophosphamide is administered intra-peritoneally. In some embodiments, the cyclophosphamide is administered through a route of administration which allows for the proper action of the cyclophosphamide by the recipient. 7.3.3 Calcineurin Inhibitors (e.g., Tacrolimus)

In some embodiments, a method of treatment described herein comprises administering a calcineurin inhibitor to a recipient. In specific embodiments, the calcineurin inhibitor is tacrolimus. Tacrolimus, as used herein, is a macrolide antibiotic which may be administered to a recipient to suppress the immune system. Tacrolimus has a mode of action similar to CyA (Cyclosporine A, another calcineurin inhibitor), and brand names of tacrolimus include Prograf®, Adport®, Advagraf®, Protopic®, Astagraf XL®, Modigraf®, and Envarsus XR®. A calcineurin inhibitor (e.g., tacrolimus) can be included in the postoperative treatment regimen to suppress the immune system and inhibit the development of Graft versus Host disease in the recipient. The postoperative treatment regimen can include a constant course followed by a tapering course of the calcineurin inhibitor (e.g., tacrolimus) administration to the recipient.

In certain embodiments, the conditioning regimen provided herein comprises administering a calcineurin inhibitor (e.g., tacrolimus) to a transplant recipient. In certain embodiments, a calcineurin inhibitor (e.g., tacrolimus) can be first administered 1 day before the transplant, 2 days before the transplant, 3 days before the transplant, 1 day and 2 days before the transplant, 1 day and 3 days before transplant, or 1 day and 2 days and 3 days before the transplant. In a specific embodiment, a calcineurin inhibitor (e.g., tacrolimus) can be first administered to a recipient 1 day before the transplant. In a specific embodiment, a calcineurin inhibitor (e.g., tacrolimus) is first administered to a recipient 2 days before the transplant. In a specific embodiment, a calcineurin inhibitor (e.g., tacrolimus) is first administered to a recipient 3 days before the transplant. In a specific embodiment, a calcineurin inhibitor (e.g., tacrolimus) is first administered to a recipient 1 day and 2 days before the transplant. In a specific embodiment, a calcineurin inhibitor (e.g., tacrolimus) is first administered to a recipient 1 day and 3 days before transplant. In a specific embodiment, a calcineurin inhibitor (e.g., tacrolimus) is first administered to a recipient 1 day and 2 days and 3 days before the transplant.

In specific embodiments, a calcineurin inhibitor (e.g., tacrolimus) is administered as soon as possible in the peri-transplant period (i.e., around the time of the transplant surgery). In specific embodiments, a calcineurin inhibitor (e.g., tacrolimus) is administered no later than 24 hours after reperfusion of the liver transplant. In certain embodiments, the postoperative treatment regimen provided herein comprises administering a calcineurin inhibitor (e.g., tacrolimus) to a transplant recipient. In certain embodiments, a calcineurin inhibitor (e.g., tacrolimus) can be administered on the day of the transplant, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, 16 days after, 17 days after, 18 days after, 19 days after, 20 days after, 21 days after, 22 days after, 23 days after, 24 days after, 25 days after, 26 days after, 27 days after, 28 days after, 29 days after, 30 days after, 1 month after, 2 months after, 3 months after, 4 months after, 5 months after, 6 months after, 7 months after, 8 months after, 9 months after, 10 months after, 11 months after, 12 months after, 13 months after, 14 months after, 15 months after, 16 months after, 17 months after, or 18 months after the transplant surgery. In some embodiments, a calcineurin inhibitor (e.g., tacrolimus) is administered on the day of the transplant. In some embodiments, a calcineurin inhibitor (e.g., tacrolimus) is administered 1 day after the transplant surgery. In some embodiments, a calcineurin inhibitor (e.g., tacrolimus) is administered 2 days after the transplant surgery. In some embodiments, a calcineurin inhibitor (e.g., tacrolimus) is administered 3 days after the transplant surgery. In some embodiments, a calcineurin inhibitor (e.g., tacrolimus) is administered 4 days after the transplant surgery. In some embodiments, a calcineurin inhibitor (e.g., tacrolimus) is administered 5 days after the transplant surgery. In some embodiments, a calcineurin inhibitor (e.g., tacrolimus) is administered 6 days after the transplant surgery. In some embodiments, a calcineurin inhibitor (e.g., tacrolimus) is administered 7 days after the transplant surgery. In some embodiments, a calcineurin inhibitor (e.g., tacrolimus) is administered 8 days after the transplant surgery. In some embodiments, a calcineurin inhibitor (e.g., tacrolimus) is administered 9 days after the transplant surgery. In some embodiments, a calcineurin inhibitor (e.g., tacrolimus) is administered 10 days after the transplant surgery. In some embodiments, a calcineurin inhibitor (e.g., tacrolimus) is administered 11 days after the transplant surgery. In some embodiments, a calcineurin inhibitor (e.g., tacrolimus) is administered 12 days after the transplant surgery. In some embodiments, a calcineurin inhibitor (e.g., tacrolimus) is administered 13 days after the transplant surgery. In some embodiments, a calcineurin inhibitor (e.g., tacrolimus) is administered 14 days after the transplant surgery. In some embodiments, a calcineurin inhibitor (e.g., tacrolimus) is administered 15 days after the transplant surgery. In some embodiments, a calcineurin inhibitor (e.g., tacrolimus) is administered 16 days after the transplant surgery. In some embodiments, a calcineurin inhibitor (e.g., tacrolimus) is administered 17 days after the transplant surgery. In some embodiments, a calcineurin inhibitor (e.g., tacrolimus) is administered 18 days after the transplant surgery. In some embodiments, a calcineurin inhibitor (e.g., tacrolimus) is administered 19 days after the transplant surgery. In some embodiments, a calcineurin inhibitor (e.g., tacrolimus) is administered 20 days after the transplant surgery. In some embodiments, a calcineurin inhibitor (e.g., tacrolimus) is administered 21 days after the transplant surgery. In some embodiments, a calcineurin inhibitor (e.g., tacrolimus) is administered 22 days after the transplant surgery. In some embodiments, a calcineurin inhibitor (e.g., tacrolimus) is administered 23 days after the transplant surgery. In some embodiments, a calcineurin inhibitor (e.g., tacrolimus) is administered 24 days after the transplant surgery. In some embodiments, a calcineurin inhibitor (e.g., tacrolimus) is administered 25 days after the transplant surgery. In some embodiments, a calcineurin inhibitor (e.g., tacrolimus) is administered 26 days after the transplant surgery. In some embodiments, a calcineurin inhibitor (e.g., tacrolimus) is administered 27 days after the transplant surgery. In some embodiments, a calcineurin inhibitor (e.g., tacrolimus) is administered 28 days after the transplant surgery. In some embodiments, a calcineurin inhibitor (e.g., tacrolimus) is administered 29 days after the transplant surgery. In some embodiments, a calcineurin inhibitor (e.g., tacrolimus) is administered 30 days after. In some embodiments, a calcineurin inhibitor (e.g., tacrolimus) is administered 1 month after the transplant surgery. In some embodiments, a calcineurin inhibitor (e.g., tacrolimus) is administered 2 months after the transplant surgery. In some embodiments, a calcineurin inhibitor (e.g., tacrolimus) is administered 3 months after the transplant surgery. In some embodiments, a calcineurin inhibitor (e.g., tacrolimus) is administered 4 months after the transplant surgery. In some embodiments, a calcineurin inhibitor (e.g., tacrolimus) is administered 5 months after the transplant surgery. In some embodiments, a calcineurin inhibitor (e.g., tacrolimus) is administered 6 months after the transplant surgery. In some embodiments, a calcineurin inhibitor (e.g., tacrolimus) is administered 7 months after the transplant surgery. In some embodiments, a calcineurin inhibitor (e.g., tacrolimus) is administered 8 months after the transplant surgery. In some embodiments, a calcineurin inhibitor (e.g., tacrolimus) is administered 9 months after the transplant surgery. In some embodiments, a calcineurin inhibitor (e.g., tacrolimus) is administered 10 months after the transplant surgery. In some embodiments, a calcineurin inhibitor (e.g., tacrolimus) is administered 11 months after the transplant surgery. In some embodiments, a calcineurin inhibitor (e.g., tacrolimus) is administered 12 months after the transplant surgery. In some embodiments, a calcineurin inhibitor (e.g., tacrolimus) is administered 13 months after the transplant surgery. In some embodiments, a calcineurin inhibitor (e.g., tacrolimus) is administered 14 months after the transplant surgery. In some embodiments, a calcineurin inhibitor (e.g., tacrolimus) is administered 15 months after the transplant surgery. In some embodiments, a calcineurin inhibitor (e.g., tacrolimus) is administered 16 months after the transplant surgery. In some embodiments, a calcineurin inhibitor (e.g., tacrolimus) is administered 17 months after the transplant surgery. In some embodiments, a calcineurin inhibitor (e.g., tacrolimus) is administered 18 months after the transplant surgery.

In certain embodiments, a single dose amount of tacrolimus can be administered. In certain embodiments, multiple dose amounts of a calcineurin inhibitor (e.g., tacrolimus) can be administered. In certain embodiments, a constant dose of a calcineurin inhibitor (e.g., tacrolimus) can be administered. In certain embodiments, a tapering course of the calcineurin inhibitor (e.g., tacrolimus) can be administered. In certain embodiments, a constant dose of the calcineurin inhibitor (e.g., tacrolimus) followed by a tapering course of the calcineurin inhibitor (e.g., tacrolimus) can be administered.

In specific embodiments, a calcineurin inhibitor (e.g., tacrolimus) may be administered at a frequency of once a day. In certain embodiments, tacrolimus can be administered to a transplant recipient at a frequency of twice a day. In certain embodiments, tacrolimus can be administered to a transplant recipient at a frequency of twice a day, beginning on the day of the transplant. In certain embodiments, a calcineurin inhibitor (e.g., tacrolimus) can be administered twice a day at a dose amount of 0.1 mg/kg/dose, 0.2 mg/kg/dose, 0.3 mg/kg/dose, 0.4 mg/kg/dose, 0.5 mg/kg/dose, 0.6 mg/kg/dose, 0.7 mg/kg/dose, 0.8 mg/kg/dose, 0.9 mg/kg/dose, 1 mg/kg/dose, 0.1-0.5 mg/kg/dose, 0.5-1 mg/kg/dose, 0.2-0.6 mg/kg/dose, 0.3-0.7 mg/kg/dose, 0.4-0.8 mg/kg/dose, or 0.1-1 mg/kg/dose. In a specific embodiment, the calcineurin inhibitor is tacrolimus and is administered to a transplant recipient orally twice a day at a dose amount of at least 0.5 mg/kg/dose. In certain embodiments, a calcineurin inhibitor (e.g., tacrolimus) is administered twice a day at a dose amount of 0.1 mg/kg/dose. In certain embodiments, a calcineurin inhibitor (e.g., tacrolimus) is administered twice a day at a dose amount of 0.2 mg/kg/dose. In certain embodiments, a calcineurin inhibitor (e.g., tacrolimus) is administered twice a day at a dose amount of 0.3 mg/kg/dose. In certain embodiments, a calcineurin inhibitor (e.g., tacrolimus) is administered twice a day at a dose amount of 0.4 mg/kg/dose. In certain embodiments, a calcineurin inhibitor (e.g., tacrolimus) is administered twice a day at a dose amount of 0.5 mg/kg/dose. In certain embodiments, a calcineurin inhibitor (e.g., tacrolimus) is administered twice a day at a dose amount of 0.6 mg/kg/dose. In certain embodiments, a calcineurin inhibitor (e.g., tacrolimus) is administered twice a day at a dose amount of 0.7 mg/kg/dose. In certain embodiments, a calcineurin inhibitor (e.g., tacrolimus) is administered twice a day at a dose amount of 0.8 mg/kg/dose. In certain embodiments, a calcineurin inhibitor (e.g., tacrolimus) is administered twice a day at a dose amount of 0.9 mg/kg/dose. In certain embodiments, a calcineurin inhibitor (e.g., tacrolimus) is administered twice a day at a dose amount of 1 mg/kg/dose. In certain embodiments, a calcineurin inhibitor (e.g., tacrolimus) is administered twice a day at a dose amount of 0.1-0.5 mg/kg/dose. In certain embodiments, a calcineurin inhibitor (e.g., tacrolimus) is administered twice a day at a dose amount of 0.5-1 mg/kg/dose. In certain embodiments, a calcineurin inhibitor (e.g., tacrolimus) is administered twice a day at a dose amount of 0.2-0.6 mg/kg/dose. In certain embodiments, a calcineurin inhibitor (e.g., tacrolimus) is administered twice a day at a dose amount of 0.3-0.7 mg/kg/dose. In certain embodiments, a calcineurin inhibitor (e.g., tacrolimus) is administered twice a day at a dose amount of 0.4-0.8 mg/kg/dose. In certain embodiments, a calcineurin inhibitor (e.g., tacrolimus) is administered twice a day at a dose amount of 0.1-1 mg/kg/dose.

In certain embodiments, a calcineurin inhibitor (e.g., tacrolimus) can be administered to a recipient at a sufficient dose amount to obtain the target trough blood levels of 1-5 ng/ml, 5-10 ng/ml, 10-15 ng/ml, 1-11 ng/ml, 2-12 ng/ml, 3-13 ng/ml, 4-14 ng/ml, 5-15 ng/ml, 6-16 ng/ml, 7-17 ng/ml, 8-18 ng/ml, 9-19 ng/ml, 10-20 ng/ml, or 15-20 ng/ml. In a specific embodiment, the target trough blood levels can be 10-15 ng/ml. In some embodiments, a calcineurin inhibitor (e.g., tacrolimus) is administered to a recipient at a sufficient dose amount to obtain the target trough blood levels of 1-5 ng/ml. In some embodiments, a calcineurin inhibitor (e.g., tacrolimus) is administered to a recipient at a sufficient dose amount to obtain the target trough blood levels of 5-10 ng/ml. In some embodiments, a calcineurin inhibitor (e.g., tacrolimus) is administered to a recipient at a sufficient dose amount to obtain the target trough blood levels of 10-15 ng/ml. In some embodiments, a calcineurin inhibitor (e.g., tacrolimus) is administered to a recipient at a sufficient dose amount to obtain the target trough blood levels of 1-11 ng/ml. In some embodiments, a calcineurin inhibitor (e.g., tacrolimus) is administered to a recipient at a sufficient dose amount to obtain the target trough blood levels of 2-12 ng/ml. In some embodiments, a calcineurin inhibitor (e.g., tacrolimus) is administered to a recipient at a sufficient dose amount to obtain the target trough blood levels of 3-13 ng/ml. In some embodiments, a calcineurin inhibitor (e.g., tacrolimus) is administered to a recipient at a sufficient dose amount to obtain the target trough blood levels of 4-14 ng/ml. In some embodiments, a calcineurin inhibitor (e.g., tacrolimus) is administered to a recipient at a sufficient dose amount to obtain the target trough blood levels of 5-15 ng/ml. In some embodiments, a calcineurin inhibitor (e.g., tacrolimus) is administered to a recipient at a sufficient dose amount to obtain the target trough blood levels of 6-16 ng/ml. In some embodiments, a calcineurin inhibitor (e.g., tacrolimus) is administered to a recipient at a sufficient dose amount to obtain the target trough blood levels of 7-17 ng/ml. In some embodiments, a calcineurin inhibitor (e.g., tacrolimus) is administered to a recipient at a sufficient dose amount to obtain the target trough blood levels of 8-18 ng/ml. In some embodiments, a calcineurin inhibitor (e.g., tacrolimus) is administered to a recipient at a sufficient dose amount to obtain the target trough blood levels of 9-19 ng/ml. In some embodiments, a calcineurin inhibitor (e.g., tacrolimus) is administered to a recipient at a sufficient dose amount to obtain the target trough blood levels of 10-20 ng/ml. In some embodiments, a calcineurin inhibitor (e.g., tacrolimus) is administered to a recipient at a sufficient dose amount to obtain the target trough blood levels of or 15-20 ng/ml. In a specific embodiment, the target trough blood levels can be 10-15 ng/ml. In some embodiments, the calcineurin inhibitor is tacrolimus and is administered to a recipient at a dose sufficient to maintain serum trough concentrations in the range of 2-15 ng/mL. In some embodiments, the calcineurin inhibitor is tacrolimus and is administered to a recipient at a dose sufficient to maintain serum trough concentrations in the range of 4-11 ng/mL. In certain embodiments, a calcineurin inhibitor (e.g., tacrolimus) is administered twice a day at a dose amount of 0.1-0.5 mg/kg/dose.

In certain embodiments, a calcineurin inhibitor (e.g., tacrolimus) can be administered to a transplant recipient in a convenient manner known in the art including subcutaneously, intravenously, intravascularly, topically, intra-arterially, intra-cranially, intramuscularly, orally, intra-orbitally, by inhalation, transdermally, or intra-peritoneally, or through a route of administration which allows for the proper action of the calcineurin inhibitor (e.g., tacrolimus) by the recipient. In a specific embodiment, the calcineurin inhibitor (e.g., tacrolimus) is administered intravenously. In some embodiments, the calcineurin inhibitor (e.g., tacrolimus) is administered subcutaneously. In some embodiments, the calcineurin inhibitor (e.g., tacrolimus) is administered intravascularly. In some embodiments, the calcineurin inhibitor (e.g., tacrolimus) is administered topically. In some embodiments, the calcineurin inhibitor (e.g., tacrolimus) is administered intra-arterially. In some embodiments, the calcineurin inhibitor (e.g., tacrolimus) is administered intra-cranially. In some embodiments, the calcineurin inhibitor (e.g., tacrolimus) is administered intramuscularly. In some embodiments, the calcineurin inhibitor (e.g., tacrolimus) is administered orally. In some embodiments, the calcineurin inhibitor (e.g., tacrolimus) is administered intra-orbitally. In some embodiments, the calcineurin inhibitor (e.g., tacrolimus) is administered by inhalation. In some embodiments, the calcineurin inhibitor (e.g., tacrolimus) is administered transdermally. In some embodiments, the calcineurin inhibitor (e.g., tacrolimus) is administered intra-peritoneally. In some embodiments, the calcineurin inhibitor (e.g., tacrolimus) is administered through a route of administration which allows for the proper action of the calcineurin inhibitor (e.g., tacrolimus) by the recipient.

In specific embodiments, a recipient is weaned off of the calcineurin inhibitor (e.g., tacrolimus) at 3 months, 4 months, 5 months, 6 months, 7 months, 8 months, 9 months, 10 months, 11 months, 12 months, 1-2 months, 2-3 months, 3-4 months, 4-5 months, 5-6 months, 6-7 months, 7-8 months, 8-9 months, 9-10 months, 10-11 months, 11-12 months, 12-13 months, 13-14 months, 14-15 months, 15-16 months, 16-17 months, 17-18 months, 18-19 months, 19-20 months, 20-21 months, 21-22 months, 22-23 months, or 23-24 months after receiving a liver transplant. In specific embodiments, a recipient is weaned off of the calcineurin inhibitor (e.g., tacrolimus) at 3 months after receiving a liver transplant. In specific embodiments, a recipient is weaned off of the calcineurin inhibitor (e.g., tacrolimus) at 4 months after receiving a liver transplant. In specific embodiments, a recipient is weaned off of the calcineurin inhibitor (e.g., tacrolimus) at 5 months after receiving a liver transplant. In specific embodiments, a recipient is weaned off of the calcineurin inhibitor (e.g., tacrolimus) at 6 months after receiving a liver transplant. In specific embodiments, a recipient is weaned off of the calcineurin inhibitor (e.g., tacrolimus) at 7 months after receiving a liver transplant. In specific embodiments, a recipient is weaned off of the calcineurin inhibitor (e.g., tacrolimus) at 8 months after receiving a liver transplant. In specific embodiments, a recipient is weaned off of the calcineurin inhibitor (e.g., tacrolimus) at 9 months after receiving a liver transplant. In specific embodiments, a recipient is weaned off of the calcineurin inhibitor (e.g., tacrolimus) at 10 months after receiving a liver transplant. In specific embodiments, a recipient is weaned off of the calcineurin inhibitor (e.g., tacrolimus) at 11 months after receiving a liver transplant. In specific embodiments, a recipient is weaned off of the calcineurin inhibitor (e.g., tacrolimus) at 12 months after receiving a liver transplant. In specific embodiments, a recipient is weaned off of the calcineurin inhibitor (e.g., tacrolimus) at 1-2 months after receiving a liver transplant. In specific embodiments, a recipient is weaned off of the calcineurin inhibitor (e.g., tacrolimus) at 2-3 months after receiving a liver transplant. In specific embodiments, a recipient is weaned off of the calcineurin inhibitor (e.g., tacrolimus) at 3-4 months after receiving a liver transplant. In specific embodiments, a recipient is weaned off of the calcineurin inhibitor (e.g., tacrolimus) at 4-5 months after receiving a liver transplant. In specific embodiments, a recipient is weaned off of the calcineurin inhibitor (e.g., tacrolimus) at 5-6 months after receiving a liver transplant. In specific embodiments, a recipient is weaned off of the calcineurin inhibitor (e.g., tacrolimus) at 6-7 months after receiving a liver transplant. In specific embodiments, a recipient is weaned off of the calcineurin inhibitor (e.g., tacrolimus) at 7-8 months after receiving a liver transplant. In specific embodiments, a recipient is weaned off of the calcineurin inhibitor (e.g., tacrolimus) at 8-9 months after receiving a liver transplant. In specific embodiments, a recipient is weaned off of the calcineurin inhibitor (e.g., tacrolimus) at 9-10 months after receiving a liver transplant. In specific embodiments, a recipient is weaned off of the calcineurin inhibitor (e.g., tacrolimus) at 10-11 months after receiving a liver transplant. In specific embodiments, a recipient is weaned off of the calcineurin inhibitor (e.g., tacrolimus) at 11-12 months after receiving a liver transplant. In specific embodiments, a recipient is weaned off of the calcineurin inhibitor (e.g., tacrolimus) at 12-13 months after receiving a liver transplant. In specific embodiments, a recipient is weaned off of the calcineurin inhibitor (e.g., tacrolimus) at 13-14 months after receiving a liver transplant. In specific embodiments, a recipient is weaned off of the calcineurin inhibitor (e.g., tacrolimus) at 14-15 months after receiving a liver transplant. In specific embodiments, a recipient is weaned off of the calcineurin inhibitor (e.g., tacrolimus) at 15-16 months after receiving a liver transplant. In specific embodiments, a recipient is weaned off of the calcineurin inhibitor (e.g., tacrolimus) at 16-17 months after receiving a liver transplant. In specific embodiments, a recipient is weaned off of the calcineurin inhibitor (e.g., tacrolimus) at 17-18 months after receiving a liver transplant. In specific embodiments, a recipient is weaned off of the calcineurin inhibitor (e.g., tacrolimus) at 18-19 months after receiving a liver transplant. In specific embodiments, a recipient is weaned off of the calcineurin inhibitor (e.g., tacrolimus) at 19-20 months after receiving a liver transplant. In specific embodiments, a recipient is weaned off of the calcineurin inhibitor (e.g., tacrolimus) at 20-21 months after receiving a liver transplant. In specific embodiments, a recipient is weaned off of the calcineurin inhibitor (e.g., tacrolimus) at 21-22 months after receiving a liver transplant. In specific embodiments, a recipient is weaned off of the calcineurin inhibitor (e.g., tacrolimus) at 22-23 months after receiving a liver transplant. In specific embodiments, a recipient is weaned off of the calcineurin inhibitor (e.g., tacrolimus) at 23-24 months after receiving a liver transplant. In specific embodiments, weaning is initiated at six months post liver transplant. In specific embodiments, weaning is only initiated if a biopsy taken from the recipient at six months is free from rejection as determined by the 2016 Banff Criteria (as described in Demetris et al., Am J Transplant. 2016 October; 16(10):2816-2835) and/or the recipient maintains stable graft function.

In specific embodiments, the dose of the calcineurin inhibitor (e.g., tacrolimus) is reduced every 2, 3, 4, 5, or 6 months after weaning begins.

In particular embodiments, a weaning protocol comprises one or more steps of reducing the frequency of administration, for example, reducing the frequency of administration from twice daily to once daily, from once daily to every other day, from once daily to three times a week, from three times a week to twice a week, from twice a week to once a week, from once a week to every other week. In some embodiments, the weaning protocol comprises reducing the frequency of administration from twice daily to once daily. In some embodiments, the weaning protocol comprises reducing the frequency of administration from once daily to every other day. In some embodiments, the weaning protocol comprises reducing the frequency of administration from once daily to three times a week. In some embodiments, the weaning protocol comprises reducing the frequency of administration from three times a week to twice a week. In some embodiments, the weaning protocol comprises reducing the frequency of administration from twice a week to once a week. In some embodiments, the weaning protocol comprises reducing the frequency of administration from once a week to every other week.

In specific embodiments, a weaning protocol comprises a step of reducing the daily dose of the calcineurin inhibitor (e.g., tacrolimus), e.g., reducing an initial daily dose by 10-20%, 20-30%, 30-40%, 40-50%, 50-60%, 60-70%, 70-80%, 80-90%, or more than 90%. In some embodiments, the weaning protocol comprises reducing the initial daily dose of the calcineurin inhibitor (e.g., tacrolimus) by 10-20%. In some embodiments, the weaning protocol comprises reducing the initial daily dose of the calcineurin inhibitor (e.g., tacrolimus) by 20-30%. In some embodiments, the weaning protocol comprises reducing the initial daily dose of the calcineurin inhibitor (e.g., tacrolimus) by 30-40%. In some embodiments, the weaning protocol comprises reducing the initial daily dose of the calcineurin inhibitor (e.g., tacrolimus) by 40-50%. In some embodiments, the weaning protocol comprises reducing the initial daily dose of the calcineurin inhibitor (e.g., tacrolimus) by 50-60%. In some embodiments, the weaning protocol comprises reducing the initial daily dose of the calcineurin inhibitor (e.g., tacrolimus) by 60-70%. In some embodiments, the weaning protocol comprises reducing the initial daily dose of the calcineurin inhibitor (e.g., tacrolimus) by 70-80%. In some embodiments, the weaning protocol comprises reducing the initial daily dose of the calcineurin inhibitor (e.g., tacrolimus) by 80-90%. In some embodiments, the weaning protocol comprises reducing the initial daily dose of the calcineurin inhibitor (e.g., tacrolimus) by more than 90%. In particular embodiments, a weaning protocol comprises a step of decreasing the daily dose of the calcineurin inhibitor (e.g., tacrolimus) by about 25%.

In particular embodiments, administration of a calcineurin inhibitor (e.g., tacrolimus) is stopped about 12-15 months, 15-18 months, 18-21 months, or 21-24 months after the transplant. In some embodiments, administration of a calcineurin inhibitor (e.g., tacrolimus) is stopped about 12-15 months after the transplant. In some embodiments, administration of a calcineurin inhibitor (e.g., tacrolimus) is stopped about 15-18 months after the transplant. In some embodiments, administration of a calcineurin inhibitor (e.g., tacrolimus) is stopped about 18-21 months after the transplant. In some embodiments, administration of a calcineurin inhibitor (e.g., tacrolimus) is stopped about 21-24 months after the transplant. In a specific embodiment, administration of a calcineurin inhibitor (e.g., tacrolimus) is stopped 18 months after the transplant. In specific embodiments, administration of a calcineurin inhibitor (e.g., tacrolimus) is only stopped if a biopsy taken from the recipient at 18 months is free from rejection as determined by the 2016 Banff Criteria as described in Demetris et al., Am J Transplant. 2016 October; 16(10):2816-2835.

An exemplary weaning protocol comprises steps of (i) reducing an initial, twice-daily dose of tacrolimus to once a day at three quarters of the daily dose over three months (e.g., from month 6 to month 9 after transplant); (ii) further reducing the dose to three times per week over three months (e.g., from month 9 to month 12 after transplant); (iii) further reducing the dose to twice a week over three months (e.g., from month 12 to month 15 after transplant); (iv) further reducing the dose to once a week over three months (e.g., from month 15 to month 18 after transplant); and (v) stopping tacrolimus administrations at 18 months after transplant.

In certain embodiments, substitute compounds can be used in the place of the calcineurin inhibitor. These compounds can include Belatacept (Nulojix), sirolimus, and everolimus. In certain embodiments, the calcineurin inhibitor can be cyclosporine (Gengraf®, Neoral®, and Sandimmune®) can be used of in place of the tacrolimus.

### 7.3.4 Steroids

A steroid, as used herein, is a compound administered to the recipient of an organ (e.g., liver) transplant to suppress the immune system. Prednisone is a corticosteroid, chemical name 17,21-dihydroxypregna-1,4-dienne-3,11,20-trione (C21H2605). Brand names of prednisone include Deltasone®, Sterapred®, Rayos®, Prednicot®, and Meticorten®.

In certain embodiments, a method of treatment provided herein comprises administering corticosteroids to a transplant recipient. In certain embodiments, corticosteroids can be administered on the day of the transplant, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, 16 days after, 17 days after, 18 days after, 19 days after, 20 days after, 21 days after, 22 days after, 23 days after, 24 days after, 25 days after, 26 days after, 27 days after, 28 days after, 29 days after, or 30 days after the transplant surgery. In some embodiments, a corticosteroid is administered on the day of transplant. In some embodiments, a corticosteroid is administered to the recipient 1 day after transplant. In some embodiments, a corticosteroid is administered to the recipient 2 days after transplant. In some embodiments, a corticosteroid is administered to the recipient 3 days after transplant. In some embodiments, a corticosteroid is administered to the recipient 4 days after transplant. In some embodiments, a corticosteroid is administered to the recipient 5 days after transplant. In some embodiments, a corticosteroid is administered to the recipient 6 days after transplant. In some embodiments, a corticosteroid is administered to the recipient 7 days after transplant. In some embodiments, a corticosteroid is administered to the recipient 8 days after transplant. In some embodiments, a corticosteroid is administered to the recipient 9 days after transplant. In some embodiments, a corticosteroid is administered to the recipient 10 days after transplant. In some embodiments, a corticosteroid is administered to the recipient 11 days after transplant. In some embodiments, a corticosteroid is administered to the recipient 12 days after transplant. In some embodiments, a corticosteroid is administered to the recipient 14 days after transplant. In some embodiments, a corticosteroid is administered to the recipient 15 days after transplant. In some embodiments, a corticosteroid is administered to the recipient 16 days after transplant. In some embodiments, a corticosteroid is administered to the recipient 17 days after transplant. In some embodiments, a corticosteroid is administered to the recipient 18 days after transplant. In some embodiments, a corticosteroid is administered to the recipient 19 days after transplant. In some embodiments, a corticosteroid is administered to the recipient 20 days after transplant. In some embodiments, a corticosteroid is administered to the recipient 21 days after transplant. In some embodiments, a corticosteroid is administered to the recipient 22 days after transplant. In some embodiments, a corticosteroid is administered to the recipient 23 days after transplant. In some embodiments, a corticosteroid is administered to the recipient 24 days after transplant. In some embodiments, a corticosteroid is administered to the recipient 25 days after transplant. In some embodiments, a corticosteroid is administered to the recipient 26 days after transplant. In some embodiments, a corticosteroid is administered to the recipient 27 days after transplant. In some embodiments, a corticosteroid is administered to the recipient 28 days after transplant. In some embodiments, a corticosteroid is administered to the recipient 29 days after transplant. In some embodiments, a corticosteroid is administered to the recipient 30 days after transplant.

In certain embodiments, a corticosteroid can be administered on the day of the transplant surgery through 5 days after, on the day of the transplant surgery through 10 days after, on the day of the transplant surgery through 15 days after, on the day of the transplant surgery through 20 days after, on the day of the transplant surgery through 25 days after, or on the day of the transplant surgery through 30 days after the transplant surgery. In specific embodiments, a corticosteroid is administered on the day of the transplant surgery and on days 2-7 after transplant surgery. In particular embodiments, the dose of corticosteroid administered to the patient is reduced from the day of the transplant to one month after transplant. In certain embodiments, a corticosteroid is administered on the day of the transplant surgery through 5 days after the transplant surgery. In certain embodiments, a corticosteroid is administered on the day of the transplant surgery through 10 days after the transplant surgery. In certain embodiments, a corticosteroid is administered on the day of the transplant surgery through 15 days after the transplant surgery. In certain embodiments, a corticosteroid is administered on the day of the transplant surgery through 20 days after the transplant surgery. In certain embodiments, a corticosteroid is administered on the day of the transplant surgery through 25 days after the transplant surgery. In certain embodiments, a corticosteroid is administered on the day of the transplant surgery through 30 days after the transplant surgery.

In certain embodiments, a single dose of the corticosteroid can be administered. In certain embodiments, multiple doses of the corticosteroid can be administered. In certain embodiments, a constant dose of corticosteroids can be administered. In certain embodiments, a pulse of corticosteroids can be administered. In certain embodiments, a tapering course of corticosteroids can be administered. In certain embodiments, a constant dose of corticosteroids followed by a tapering course of corticosteroids can be administered. In certain embodiments, a constant dose of corticosteroids, with pulses of corticosteroids, and a tapering course of corticosteroids can be administered.

In certain embodiments, the corticosteroid can be administered to a transplant recipient at a dose amount of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, or 3.0 mg/kg. In some embodiments, the corticosteroid is administered to a transplant recipient at a dose amount of 0.1 mg/kg. In some embodiments, the corticosteroid is administered to a transplant recipient at a dose amount of 0.2 mg/kg. In some embodiments, the corticosteroid is administered to a transplant recipient at a dose amount of 0.3 mg/kg. In some embodiments, the corticosteroid is administered to a transplant recipient at a dose amount of 0.4 mg/kg. In some embodiments, the corticosteroid is administered to a transplant recipient at a dose amount of 0.5 mg/kg. In some embodiments, the corticosteroid is administered to a transplant recipient at a dose amount of 0.6 mg/kg. In some embodiments, the corticosteroid is administered to a transplant recipient at a dose amount of 0.7 mg/kg. In some embodiments, the corticosteroid is administered to a transplant recipient at a dose amount of 0.8 mg/kg. In some embodiments, the corticosteroid is administered to a transplant recipient at a dose amount of 0.9 mg/kg. In some embodiments, the corticosteroid is administered to a transplant recipient at a dose amount of 1.0 mg/kg. In some embodiments, the corticosteroid is administered to a transplant recipient at a dose amount of 1.1 mg/kg. In some embodiments, the corticosteroid is administered to a transplant recipient at a dose amount of 1.2 mg/kg. In some embodiments, the corticosteroid is administered to a transplant recipient at a dose amount of 1.3 mg/kg. In some embodiments, the corticosteroid is administered to a transplant recipient at a dose amount of 1.4 mg/kg. In some embodiments, the corticosteroid is administered to a transplant recipient at a dose amount of 1.5 mg/kg. In some embodiments, the corticosteroid is administered to a transplant recipient at a dose amount of 1.6 mg/kg. In some embodiments, the corticosteroid is administered to a transplant recipient at a dose amount of 1.7 mg/kg. In some embodiments, the corticosteroid is administered to a transplant recipient at a dose amount of 1.8 mg/kg. In some embodiments, the corticosteroid is administered to a transplant recipient at a dose amount of 1.9 mg/kg. In some embodiments, the corticosteroid is administered to a transplant recipient at a dose amount of 2.0 mg/kg. In some embodiments, the corticosteroid is administered to a transplant recipient at a dose amount of 2.1 mg/kg. In some embodiments, the corticosteroid is administered to a transplant recipient at a dose amount of 2.2 mg/kg. In some embodiments, the corticosteroid is administered to a transplant recipient at a dose amount of 2.3 mg/kg. In some embodiments, the corticosteroid is administered to a transplant recipient at a dose amount of 2.4 mg/kg. In some embodiments, the corticosteroid is administered to a transplant recipient at a dose amount of 2.5 mg/kg. In some embodiments, the corticosteroid is administered to a transplant recipient at a dose amount of 2.6 mg/kg. In some embodiments, the corticosteroid is administered to a transplant recipient at a dose amount of 2.7 mg/kg. In some embodiments, the corticosteroid is administered to a transplant recipient at a dose amount of 2.8 mg/kg. In some embodiments, the corticosteroid is administered to a transplant recipient at a dose amount of 2.9 mg/kg. In some embodiments, the corticosteroid is administered to a transplant recipient at a dose amount of 3.0 mg/kg.

In certain embodiments, the corticosteroid can be administered to a transplant recipient at a pulsed dose amount of 100 mg/dose, 200 mg/dose, 300 mg/dose, 400 mg/dose, 500 mg/dose, 600 mg/dose, 700 mg/dose, 800 mg/dose, 900 mg/dose, or 1000 mg/dose. In some embodiments, the corticosteroid is administered to a transplant recipient at a pulsed dose amount of 100 mg/dose. In some embodiments, the corticosteroid is administered to a transplant recipient at a pulsed dose amount of 200 mg/dose. In some embodiments, the corticosteroid is administered to a transplant recipient at a pulsed dose amount of 300 mg/dose. In some embodiments, the corticosteroid is administered to a transplant recipient at a pulsed dose amount of 400 mg/dose. In some embodiments, the corticosteroid is administered to a transplant recipient at a pulsed dose amount of 500 mg/dose. In some embodiments, the corticosteroid is administered to a transplant recipient at a pulsed dose amount of 600 mg/dose. In some embodiments, the corticosteroid is administered to a transplant recipient at a pulsed dose amount of 700 mg/dose. In some embodiments, the corticosteroid is administered to a transplant recipient at a pulsed dose amount of 800 mg/dose. In some embodiments, the corticosteroid is administered to a transplant recipient at a pulsed dose amount of 900 mg/dose. In some embodiments, the corticosteroid is administered to a transplant recipient at a pulsed dose amount of 1000 mg/dose.

In certain embodiments, a pulse of the corticosteroid can be administered on the day of the transplant, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, the day of and 1 day and 2 days after, 3 days and 4 days and 5 days after, 6 days and 7 days and 8 days after, 9 days and 10 days and 11 days after, 10 days and 11 days and 12 days after, 11 days and 12 days and 13 days after, or 13 days and 14 days and 15 days after the transplant. In a specific embodiment, a pulse of the corticosteroid is administered the day of the transplant. In a specific embodiment, a pulse of corticosteroid is administered 10 days and 11 days and 12 days after the transplant. In certain embodiments, a pulse of the corticosteroid is administered on the day of the transplant. In certain embodiments, a pulse of the corticosteroid is administered 1 day after the transplant. In certain embodiments, a pulse of the corticosteroid is administered 2 days after the transplant. In certain embodiments, a pulse of the corticosteroid is administered 3 days after the transplant. In certain embodiments, a pulse of the corticosteroid is administered 4 days after the transplant. In certain embodiments, a pulse of the corticosteroid is administered 5 days after the transplant. In certain embodiments, a pulse of the corticosteroid is administered 6 days after the transplant. In certain embodiments, a pulse of the corticosteroid is administered 7 days after the transplant. In certain embodiments, a pulse of the corticosteroid is administered 8 days after the transplant. In certain embodiments, a pulse of the corticosteroid is administered 9 days after the transplant. In certain embodiments, a pulse of the corticosteroid is administered 10 days after the transplant. In certain embodiments, a pulse of the corticosteroid is administered 11 days after the transplant. In certain embodiments, a pulse of the corticosteroid is administered 12 days after the transplant. In certain embodiments, a pulse of the corticosteroid is administered 13 days after the transplant. In certain embodiments, a pulse of the corticosteroid is administered 14 days after the transplant. In certain embodiments, a pulse of the corticosteroid is administered 15 days after the transplant. In certain embodiments, a pulse of the corticosteroid is administered the day of and 1 day and 2 days after the transplant. In certain embodiments, a pulse of the corticosteroid is administered 3 days and 4 days and 5 days after the transplant. In certain embodiments, a pulse of the corticosteroid is administered 6 days and 7 days and 8 days after the transplant. In certain embodiments, a pulse of the corticosteroid is administered 9 days and 10 days and 11 days after the transplant. In certain embodiments, a pulse of the corticosteroid is administered 10 days and 11 days and 12 days after the transplant. In certain embodiments, a pulse of the corticosteroid is administered 11 days and 12 days and 13 days after the transplant. In certain embodiments, a pulse of the corticosteroid is administered or 13 days and 14 days and 15 days after the transplant.

In certain embodiments, the steroid can be administered to a transplant recipient at a constant dose. The duration of time a constant dose is administered can be 1 day, 2 days, 3 days, 4 days, 5 days, 6 days, 7 days, 8 days, 9 days, or 10 days. In some embodiments, the steroid is administered to a transplant recipient at a constant dose for 1 day. In some embodiments, the steroid is administered to a transplant recipient at a constant dose for 2 days. In some embodiments, the steroid is administered to a transplant recipient at a constant dose for 3 days. In some embodiments, the steroid is administered to a transplant recipient at a constant dose for 4 days. In some embodiments, the steroid is administered to a transplant recipient at a constant dose for 5 days. In some embodiments, the steroid is administered to a transplant recipient at a constant dose for 6 days. In some embodiments, the steroid is administered to a transplant recipient at a constant dose for 7 days. In some embodiments, the steroid is administered to a transplant recipient at a constant dose for 8 days. In some embodiments, the steroid is administered to a transplant recipient at a constant dose for 9 days. In some embodiments, the steroid is administered to a transplant recipient at a constant dose for 10 days.

In certain embodiments, the corticosteroids administered to a transplant recipient can be tapered to discontinuation. In certain embodiments, this tapering course can take place over 5 days, 6 days, 7 days, 8 days, 9 days, 10 days, 11 days, 12 days, 13 days, 14 days 15 days, 16 days, 17 days, 18 days, 19 days, 20 days, 21 days, 22 days, 23 days, 24 days 25 days, 26 days, 27 days, 28 days, 29 days, or 30 days. In a specific embodiment, the tapering course can take place over 10 days. In certain embodiments, the tapering course takes place over 5 days. In some embodiments, the tapering course takes place over 6 days. In some embodiments, the tapering course takes place over 7 days. In some embodiments, the tapering course takes place over 8 days. In some embodiments, the tapering course takes place over 9 days. In some embodiments, the tapering course takes place over 10 days. In some embodiments, the tapering course takes place over 11 days. In some embodiments, the tapering course takes place over 12 days. In some embodiments, the tapering course takes place over 13 days. In some embodiments, the tapering course takes place over 14 days. In certain embodiments, the tapering course takes place over 15 days. In some embodiments, the tapering course takes place over 16 days. In some embodiments, the tapering course takes place over 17 days. In some embodiments, the tapering course takes place over 18 days. In some embodiments, the tapering course takes place over 19 days. In some embodiments, the tapering course takes place over 20 days. In some embodiments, the tapering course takes place over 21 days. In some embodiments, the tapering course takes place over 22 days. In some embodiments, the tapering course takes place over 23 days. In some embodiments, the tapering course takes place over 24 days. In some embodiments, the tapering course takes place over 25 days. In some embodiments, the tapering course takes place over 26 days. In some embodiments, the tapering course takes place over 27 days. In some embodiments, the tapering course takes place over 28 days. In some embodiments, the tapering course takes place over 29 days. In some embodiments, the tapering course takes place over 30 days.

In certain embodiments, for the tapering course, the corticosteroids administered to the recipient can be reduced by 0.01 mg/kg per day, 0.02 mg/kg per day, 0.03 mg/kg per day, 0.04 mg/kg per day, 0.05 mg/kg per day, 0.06 mg/kg per day, 0.07 mg/kg per day, 0.08 mg/kg per day, 0.09 mg/kg per day, 0.1 mg/kg per day, 0.2 mg/kg per day, 0.3 mg/kg per day, 0.4 mg/kg per day, 0.5 mg/kg per day, 0.6 mg/kg per day, 0.7 mg/kg per day, 0.8 mg/kg per day, 0.9 mg/kg per day, 1.0 mg/kg per day, 1.1 mg/kg per day, 1.2 mg/kg per day, 1.3 mg/kg per day, 1.4 mg/kg per day, 1.5 mg/kg per day, 1.6 mg/kg per day, 1.7 mg/kg per day, 1.8 mg/kg per day, 1.9 mg/kg per day, or 2.0 mg/kg per day. In certain embodiments, for the tapering course, the corticosteroids administered to the recipient is reduced by 0.01 mg/kg per day. In certain embodiments, for the tapering course, the corticosteroids administered to the recipient is reduced by 0.02 mg/kg per day. In certain embodiments, for the tapering course, the corticosteroids administered to the recipient is reduced by 0.03 mg/kg per day. In certain embodiments, for the tapering course, the corticosteroids administered to the recipient is reduced by 0.04 mg/kg per day. In certain embodiments, for the tapering course, the corticosteroids administered to the recipient is reduced by 0.05 mg/kg per day. In certain embodiments, for the tapering course, the corticosteroids administered to the recipient is reduced by 0.06 mg/kg per day. In certain embodiments, for the tapering course, the corticosteroids administered to the recipient is reduced by 0.07 mg/kg per day. In certain embodiments, for the tapering course, the corticosteroids administered to the recipient is reduced by 0.08 mg/kg per day. In certain embodiments, for the tapering course, the corticosteroids administered to the recipient is reduced by 0.09 mg/kg per day. In certain embodiments, for the tapering course, the corticosteroids administered to the recipient is reduced by 0.1 mg/kg per day. In certain embodiments, for the tapering course, the corticosteroids administered to the recipient is reduced by 0.2 mg/kg per day. In certain embodiments, for the tapering course, the corticosteroids administered to the recipient is reduced by 0.3 mg/kg per day. In certain embodiments, for the tapering course, the corticosteroids administered to the recipient is reduced by 0.4 mg/kg per day. In certain embodiments, for the tapering course, the corticosteroids administered to the recipient is reduced by 0.5 mg/kg per day. In certain embodiments, for the tapering course, the corticosteroids administered to the recipient is reduced by 0.6 mg/kg per day. In certain embodiments, for the tapering course, the corticosteroids administered to the recipient is reduced by 0.7 mg/kg per day. In certain embodiments, for the tapering course, the corticosteroids administered to the recipient is reduced by 0.8 mg/kg per day. In certain embodiments, for the tapering course, the corticosteroids administered to the recipient is reduced by 0.9 mg/kg per day. In certain embodiments, for the tapering course, the corticosteroids administered to the recipient is reduced by 1.0 mg/kg per day. In certain embodiments, for the tapering course, the corticosteroids administered to the recipient is reduced by 1.1 mg/kg per day. In certain embodiments, for the tapering course, the corticosteroids administered to the recipient is reduced by 1.2 mg/kg per day. In certain embodiments, for the tapering course, the corticosteroids administered to the recipient is reduced by 1.3 mg/kg per day. In certain embodiments, for the tapering course, the corticosteroids administered to the recipient is reduced by 1.4 mg/kg per day. In certain embodiments, for the tapering course, the corticosteroids administered to the recipient is reduced by 1.5 mg/kg per day. In certain embodiments, for the tapering course, the corticosteroids administered to the recipient is reduced by 1.6 mg/kg per day. In certain embodiments, for the tapering course, the corticosteroids administered to the recipient is reduced by 1.7 mg/kg per day. In certain embodiments, for the tapering course, the corticosteroids administered to the recipient is reduced by 1.8 mg/kg per day. In certain embodiments, for the tapering course, the corticosteroids administered to the recipient is reduced by 1.9 mg/kg per day. In certain embodiments, for the tapering course, the corticosteroids administered to the recipient is reduced by 2.0 mg/kg per day.

An exemplary steroid dosing regimen comprises administering the following doses to a transplant recipient: (i) 1000 mg methylprednisolone intravenously on the day of transplant surgery; (ii) 250 mg oral steroid on day 2 after transplant surgery, (iii) 125 mg oral steroid on day 3 after transplant surgery; (iv) 75 mg oral steroid on day 4 after transplant surgery; (v) 60 mg oral steroid on each of days 5-7 after the transplant surgery; and (vi) 20 mg oral steroid daily from day 7 to one month after transplant surgery.

In certain embodiments, the corticosteroid can be administered to a transplant recipient in a convenient manner known in the art including subcutaneously, intravenously, intravascularly, topically, intra-arterially, intra-cranially, intramuscularly, orally, intra-orbitally, by inhalation, transdermally, or intra-peritoneally, or through a route of administration which allows for the proper action of the corticosteroid by the recipient. In a specific embodiment, the corticosteroid is administered intravenously. In some embodiments, the corticosteroid is administered subcutaneously. In some embodiments, the corticosteroid is administered intravascularly. In some embodiments, the corticosteroid is administered topically. In some embodiments, the corticosteroid is administered intra-arterially. In some embodiments, the corticosteroid is administered intra-cranially. In some embodiments, the corticosteroid is administered intramuscularly. In some embodiments, the corticosteroid is administered orally. In some embodiments, the corticosteroid is administered intra-orbitally. In some embodiments, the corticosteroid is administered by inhalation. In some embodiments, the corticosteroid is administered transdermally. In some embodiments, the corticosteroid is administered intra-peritoneally. In some embodiments, the corticosteroid is administered through a route of administration which allows for the proper action of the corticosteroid by the recipient.

Examples of steroids that may be used in a method described herein include Deltison®, Prednisolone EQL Pharma, and Prednisolon Alternova.

In a specific embodiment, the corticosteroid administered in the postoperative treatment regimen can be prednisone. In a specific embodiment, the pulsed corticosteroid administered in the postoperative treatment regimen can be methylprednisone. In certain embodiments, the administration of corticosteroids can be modified as described herein.

### 7.3.5 Cyclosporine

Cyclosporine (CyA) can be included in the postoperative treatment regimen to suppress the immune system and inhibit the development of Graft versus Host disease in the recipient. The postoperative treatment regimen can include a constant course followed by a tapering course of CyA administration to the recipient. In certain embodiments, cyclosporine can be used in place of a calcineurin inhibitor (e.g., tacrolimus) in a method provided herein. In some embodiments, cyclosporine may be used in a recipient who experienced adverse events with a calcineurin inhibitor. In specific embodiments, cyclosporine may be used in a recipient who experienced tacrolimus-related adverse events.

In certain embodiments, the postoperative treatment regimen provided herein comprises administering CyA to a transplant recipient. In certain embodiments, CyA can be administered on the day of the transplant, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, 16 days after, 17 days after, 18 days after, 19 days after, 20 days after, 21 days after, 22 days after, 23 days after, 24 days after, 25 days after, 26 days after, 27 days after, 28 days after, 29 days after, 30 days after, 1 month after, 2 months after, 3 months after, 4 months after, 5 months after, 6 months after, 7 months after, 8 months after, 9 months after, 10 months after, 11 months after, 12 months after, 13 months after, 14 months after, 15 months after, 16 months after, 17 months after, or 18 months after the transplant surgery. In certain embodiments, CyA is administered on the day of the transplant. In certain embodiments, CyA is administered 1 day after the transplant surgery. In certain embodiments, CyA is administered 2 days after the transplant surgery. In certain embodiments, CyA is administered 3 days after the transplant surgery. In certain embodiments, CyA is administered 4 days after the transplant surgery. In certain embodiments, CyA is administered 5 days after the transplant surgery. In certain embodiments, CyA is administered 6 days after the transplant surgery. In certain embodiments, CyA is administered 7 days after the transplant surgery. In certain embodiments, CyA is administered 8 days after the transplant surgery. In certain embodiments, CyA is administered 9 days after the transplant surgery. In certain embodiments, CyA is administered 10 days after the transplant surgery. In certain embodiments, CyA is administered 11 days after the transplant surgery. In certain embodiments, CyA is administered 12 days after the transplant surgery. In certain embodiments, CyA is administered 13 days after the transplant surgery. In certain embodiments, CyA is administered 14 days after the transplant surgery. In certain embodiments, CyA is administered 15 days after the transplant surgery. In certain embodiments, CyA is administered 16 days after the transplant surgery. In certain embodiments, CyA is administered 17 days after the transplant surgery. In certain embodiments, CyA is administered 18 days after the transplant surgery. In certain embodiments, CyA is administered 19 days after the transplant surgery. In certain embodiments, CyA is administered 20 days after the transplant surgery. In certain embodiments, CyA is administered 21 days after the transplant surgery. In certain embodiments, CyA is administered 22 days after the transplant surgery. In certain embodiments, CyA is administered 23 days after the transplant surgery. In certain embodiments, CyA is administered 24 days after the transplant surgery. In certain embodiments, CyA is administered 25 days after the transplant surgery. In certain embodiments, CyA is administered 26 days after the transplant surgery. In certain embodiments, CyA is administered 27 days after the transplant surgery. In certain embodiments, CyA is administered 28 days after the transplant surgery. In certain embodiments, CyA is administered 29 days after the transplant surgery. In certain embodiments, CyA is administered 30 days after. In certain embodiments, CyA is administered 1 month after the transplant surgery. In certain embodiments, CyA is administered 2 months after the transplant surgery. In certain embodiments, CyA is administered 3 months after the transplant surgery. In certain embodiments, CyA is administered 4 months after the transplant surgery. In certain embodiments, CyA is administered 5 months after the transplant surgery. In certain embodiments, CyA is administered 6 months after the transplant surgery. In certain embodiments, CyA is administered 7 months after the transplant surgery. In certain embodiments, CyA is administered 8 months after the transplant surgery. In certain embodiments, CyA is administered 9 months after the transplant surgery. In certain embodiments, CyA is administered 10 months after the transplant surgery. In certain embodiments, CyA is administered 11 months after the transplant surgery. In certain embodiments, CyA is administered 12 months after the transplant surgery. In certain embodiments, CyA is administered 13 months after the transplant surgery. In certain embodiments, CyA is administered 14 months after the transplant surgery. In certain embodiments, CyA is administered 15 months after the transplant surgery. In certain embodiments, CyA is administered 16 months after the transplant surgery. In certain embodiments, CyA is administered 17 months after the transplant surgery. In certain embodiments, CyA is administered 18 months after the transplant surgery.

In certain embodiments, a single dose amount of CyA can be administered. In certain embodiments, multiple dose amounts of the CyA can be administered. In certain embodiments, a constant dose of CyA can be administered. In certain embodiments, a tapering course of CyA can be administered. In certain embodiments, a constant dose of CyA followed by a tapering course of CyA can be administered postoperatively.

In certain embodiments, CyA can be administered to a transplant recipient at a dose amount of 2 mg/kg/day, 2.5 mg/kg/day, 3 mg/kg/day, 3.5 mg/kg/day, 4 mg/kg/day, 4.5 mg/kg/day, 5 mg/kg/day, 5.5 mg/kg/day, 6 mg/kg/day, 6.5 mg/kg/day, 7 mg/kg/day, 7.5 mg/kg/day, 8 mg/kg/day, 8.5 mg/kg/day, 9 mg/kg/day, 9.5 mg/kg/day, 10 mg/kg/day, 10.5 mg/kg/day, 11 mg/kg/day, 11.5 mg/kg/day, 12 mg/kg/day, 12.5 mg/kg/day, 13 mg/kg/day, 13.5 mg/kg/day, 14 mg/kg/day, 14.5 mg/kg/day, 15 mg/kg/day, 15.5 mg/kg/day, 16 mg/kg/day, 16.5 mg/kg/day, 17 mg/kg/day, 17.5 mg/kg/day, 18 mg/kg/day, 2-6 mg/kg/day, 3-7 mg/kg/day, 4-8 mg/kg/day, 5-9 mg/kg/day, 6-10 mg/kg/day, 7-11 mg/kg/day, 8-12 mg/kg/day, 9-13 mg/kg/day, 10-14 mg/kg/day, 11-15 mg/kg/day, 12-16 mg/kg/day, 13-17 mg/kg/day, or 14-18 mg/kg/day. In some embodiments, CyA is administered to a transplant recipient at a dose amount of 2 mg/kg/day. In some embodiments, CyA is administered to a transplant recipient at a dose amount of 2.5 mg/kg/day. In some embodiments, CyA is administered to a transplant recipient at a dose amount of 3 mg/kg/day. In some embodiments, CyA is administered to a transplant recipient at a dose amount of 3.5 mg/kg/day. In some embodiments, CyA is administered to a transplant recipient at a dose amount of 4 mg/kg/day. In some embodiments, CyA is administered to a transplant recipient at a dose amount of 4.5 mg/kg/day. In some embodiments, CyA is administered to a transplant recipient at a dose amount of 5 mg/kg/day. In some embodiments, CyA is administered to a transplant recipient at a dose amount of 5.5 mg/kg/day. In some embodiments, CyA is administered to a transplant recipient at a dose amount of 6 mg/kg/day. In some embodiments, CyA is administered to a transplant recipient at a dose amount of 6.5 mg/kg/day. In some embodiments, CyA is administered to a transplant recipient at a dose amount of 7 mg/kg/day. In some embodiments, CyA is administered to a transplant recipient at a dose amount of 7.5 mg/kg/day. In some embodiments, CyA is administered to a transplant recipient at a dose amount of 8 mg/kg/day. In some embodiments, CyA is administered to a transplant recipient at a dose amount of 8.5 mg/kg/day. In some embodiments, CyA is administered to a transplant recipient at a dose amount of 9 mg/kg/day. In some embodiments, CyA is administered to a transplant recipient at a dose amount of 9.5 mg/kg/day. In some embodiments, CyA is administered to a transplant recipient at a dose amount of 10 mg/kg/day. In some embodiments, CyA is administered to a transplant recipient at a dose amount of 10.5 mg/kg/day. In some embodiments, CyA is administered to a transplant recipient at a dose amount of 11 mg/kg/day. In some embodiments, CyA is administered to a transplant recipient at a dose amount of 11.5 mg/kg/day. In some embodiments, CyA is administered to a transplant recipient at a dose amount of 12 mg/kg/day. In some embodiments, CyA is administered to a transplant recipient at a dose amount of 12.5 mg/kg/day. In some embodiments, CyA is administered to a transplant recipient at a dose amount of 13 mg/kg/day. In some embodiments, CyA is administered to a transplant recipient at a dose amount of 13.5 mg/kg/day. In some embodiments, CyA is administered to a transplant recipient at a dose amount of 14 mg/kg/day. In some embodiments, CyA is administered to a transplant recipient at a dose amount of 14.5 mg/kg/day. In some embodiments, CyA is administered to a transplant recipient at a dose amount of 15 mg/kg/day. In some embodiments, CyA is administered to a transplant recipient at a dose amount of 15.5 mg/kg/day. In some embodiments, CyA is administered to a transplant recipient at a dose amount of 16 mg/kg/day. In some embodiments, CyA is administered to a transplant recipient at a dose amount of 16.5 mg/kg/day. In some embodiments, CyA is administered to a transplant recipient at a dose amount of 17 mg/kg/day. In some embodiments, CyA is administered to a transplant recipient at a dose amount of 17.5 mg/kg/day. In some embodiments, CyA is administered to a transplant recipient at a dose amount of 18 mg/kg/day. In some embodiments, CyA is administered to a transplant recipient at a dose amount of 2-6 mg/kg/day. In some embodiments, CyA is administered to a transplant recipient at a dose amount of 3-7 mg/kg/day. In some embodiments, CyA is administered to a transplant recipient at a dose amount of 4-8 mg/kg/day. In some embodiments, CyA is administered to a transplant recipient at a dose amount of 5-9 mg/kg/day. In some embodiments, CyA is administered to a transplant recipient at a dose amount of 6-10 mg/kg/day. In some embodiments, CyA is administered to a transplant recipient at a dose amount of 7-11 mg/kg/day. In some embodiments, CyA is administered to a transplant recipient at a dose amount of 8-12 mg/kg/day. In some embodiments, CyA is administered to a transplant recipient at a dose amount of 9-13 mg/kg/day. In some embodiments, CyA is administered to a transplant recipient at a dose amount of 10-14 mg/kg/day. In some embodiments, CyA is administered to a transplant recipient at a dose amount of 11-15 mg/kg/day. In some embodiments, CyA is administered to a transplant recipient at a dose amount of 12-16 mg/kg/day. In some embodiments, CyA is administered to a transplant recipient at a dose amount of 13-17 mg/kg/day. In some embodiments, CyA is administered to a transplant recipient at a dose amount of 14-18 mg/kg/day.

In certain embodiments, CyA can be administered postoperatively to a recipient at a sufficient dose amount to obtain the target trough blood levels of 100-200 ng/ml, 125-225 ng/ml, 150-250 ng/ml, 175-275 ng/ml, 200-300 ng/ml, 225-325 ng/ml, 250-350 ng/ml, 275-375 ng/ml, 300-400 ng/ml, 325-425 ng/ml, 350-450 ng/ml, 375-475 ng/ml, or 400-500 ng/ml. In some embodiments, CyA is administered postoperatively to a recipient at a sufficient dose amount to obtain the target trough blood levels of 100-200 ng/ml. In some embodiments, CyA is administered postoperatively to a recipient at a sufficient dose amount to obtain the target trough blood levels of 125-225 ng/ml. In some embodiments, CyA is administered postoperatively to a recipient at a sufficient dose amount to obtain the target trough blood levels of 150-250 ng/ml. In some embodiments, CyA is administered postoperatively to a recipient at a sufficient dose amount to obtain the target trough blood levels of 175-275 ng/ml. In some embodiments, CyA is administered postoperatively to a recipient at a sufficient dose amount to obtain the target trough blood levels of 200-300 ng/ml. In some embodiments, CyA is administered postoperatively to a recipient at a sufficient dose amount to obtain the target trough blood levels of 225-325 ng/ml. In some embodiments, CyA is administered postoperatively to a recipient at a sufficient dose amount to obtain the target trough blood levels of 250-350 ng/ml. In some embodiments, CyA is administered postoperatively to a recipient at a sufficient dose amount to obtain the target trough blood levels of 275-375 ng/ml. In some embodiments, CyA is administered postoperatively to a recipient at a sufficient dose amount to obtain the target trough blood levels of 300-400 ng/ml. In some embodiments, CyA is administered postoperatively to a recipient at a sufficient dose amount to obtain the target trough blood levels of 325-425 ng/ml. In some embodiments, CyA is administered postoperatively to a recipient at a sufficient dose amount to obtain the target trough blood levels of 350-450 ng/ml. In some embodiments, CyA is administered postoperatively to a recipient at a sufficient dose amount to obtain the target trough blood levels of 375-475 ng/ml. In some embodiments, CyA is administered postoperatively to a recipient at a sufficient dose amount to obtain the target trough blood levels of 400-500 ng/ml. In a specific embodiment, the target trough blood levels can be 250-350 ng/ml.

In certain embodiments, CyA can be administered to a transplant recipient at a constant dose. The duration of time a constant dose is administered can be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after the transplant. In some embodiments, CyA is administered to a transplant recipient at a constant dose for 1 day after the transplant. In some embodiments, CyA is administered to a transplant recipient at a constant dose for 2 days after the transplant. In some embodiments, CyA is administered to a transplant recipient at a constant dose for 3 days after the transplant. In some embodiments, CyA is administered to a transplant recipient at a constant dose for 4 days after the transplant. In some embodiments, CyA is administered to a transplant recipient at a constant dose for 5 days after the transplant. In some embodiments, CyA is administered to a transplant recipient at a constant dose for 6 days after the transplant. In some embodiments, CyA is administered to a transplant recipient at a constant dose for 7 days after the transplant. In some embodiments, CyA is administered to a transplant recipient at a constant dose for 8 days after the transplant. In some embodiments, CyA is administered to a transplant recipient at a constant dose for 9 days after the transplant. In some embodiments, CyA is administered to a transplant recipient at a constant dose for 10 days after the transplant. In some embodiments, CyA is administered to a transplant recipient at a constant dose for 11 days after the transplant. In some embodiments, CyA is administered to a transplant recipient at a constant dose for 12 days after the transplant. In some embodiments, CyA is administered to a transplant recipient at a constant dose for 13 days after the transplant. In some embodiments, CyA is administered to a transplant recipient at a constant dose for 14 days after the transplant. In some embodiments, CyA is administered to a transplant recipient at a constant dose for 15 days after the transplant. In some embodiments, CyA is administered to a transplant recipient at a constant dose for 16 days after the transplant. In some embodiments, CyA is administered to a transplant recipient at a constant dose for 17 days after the transplant. In some embodiments, CyA is administered to a transplant recipient at a constant dose for 18 days after the transplant. In some embodiments, CyA is administered to a transplant recipient at a constant dose for 19 days after the transplant. In some embodiments, CyA is administered to a transplant recipient at a constant dose for 20 days after the transplant. In some embodiments, CyA is administered to a transplant recipient at a constant dose for 21 days after the transplant. In some embodiments, CyA is administered to a transplant recipient at a constant dose for 22 days after the transplant. In some embodiments, CyA is administered to a transplant recipient at a constant dose for 23 days after the transplant. In some embodiments, CyA is administered to a transplant recipient at a constant dose for 24 days after the transplant. In some embodiments, CyA is administered to a transplant recipient at a constant dose for 25 days after the transplant. In some embodiments, CyA is administered to a transplant recipient at a constant dose for 26 days after the transplant. In some embodiments, CyA is administered to a transplant recipient at a constant dose for 27 days after the transplant. In some embodiments, CyA is administered to a transplant recipient at a constant dose for 28 days after the transplant. In some embodiments, CyA is administered to a transplant recipient at a constant dose for 29 days after the transplant. In some embodiments, CyA is administered to a transplant recipient at a constant dose for 30 days after the transplant.

In certain embodiments, CyA is administered to a transplant recipient can be tapered to discontinuation. In certain embodiments, this tapering course can take place over 1 month, 2 months, 3 months, 4 months, 5 months, 6 months, 7 months, 8 months, 9 months, 10 months, 11 months, 12 months, 13 months, 14 months, 15 months, 16 months, 17 months, or 18 months after the transplant. In certain embodiments, the tapering course takes place over 1 month after the transplant. In certain embodiments, the tapering course takes place over 2 months after the transplant. In certain embodiments, the tapering course takes place over 3 months after the transplant. In certain embodiments, the tapering course takes place over 4 months after the transplant. In certain embodiments, the tapering course takes place over 5 months after the transplant. In certain embodiments, the tapering course takes place over 6 months after the transplant. In certain embodiments, the tapering course takes place over 7 months after the transplant. In certain embodiments, the tapering course takes place over 8 months after the transplant. In certain embodiments, the tapering course takes place over 9 months after the transplant. In certain embodiments, the tapering course takes place over 10 months after the transplant. In certain embodiments, the tapering course takes place over 11 months after the transplant. In certain embodiments, the tapering course takes place over 12 months after the transplant. In certain embodiments, the tapering course takes place over 13 months after the transplant. In certain embodiments, the tapering course takes place over 14 months after the transplant. In certain embodiments, the tapering course takes place over 15 months after the transplant. In certain embodiments, the tapering course takes place over 16 months after the transplant. In certain embodiments, the tapering course takes place over 17 months after the transplant. In certain embodiments, the tapering course takes place over 18 months after the transplant.

In certain embodiments, CyA can be administered to a transplant recipient in a convenient manner known in the art including subcutaneously, intravenously, intravascularly, topically, intra-arterially, intra-cranially, intramuscularly, orally, intra-orbitally, by inhalation, transdermally, or intra-peritoneally, or through a route of administration which allows for the proper action of the CyA by the recipient. In a specific embodiment, CyA is administered intravenously. In some embodiments, CyA is administered subcutaneously. In some embodiments, CyA is administered intravascularly. In some embodiments, CyA is administered topically. In some embodiments, CyA is administered intra-arterially. In some embodiments, CyA is administered intra-cranially. In some embodiments, CyA is administered intramuscularly. In some embodiments, CyA is administered orally. In some embodiments, CyA is administered intra-orbitally. In some embodiments, CyA is administered by inhalation. In some embodiments, CyA is administered transdermally. In some embodiments, CyA is administered intra-peritoneally. In some embodiments, CyA is administered through a route of administration which allows for the proper action of the CyA by the recipient.

In certain embodiments, substitute compounds can be used in the place of CyA. These compounds can include tacrolimus (Prograf®, Adport®, Advagraf®, Envarsus®, Modigraf®, Astagraf®), Belatacept (Nulojix®), sirolimus, and everolimus. In specific embodiments, Belatacept can be used to suppress the immune system in the recipient.

7.3.6 Antimetabolite (e.g. Mycophenolate) Therapy

In some aspects, a method of treatment provided herein comprises administering standard-of-care-antimetabolite therapy to a recipient. In some embodiments, the standard-of-care antimetabolite therapy is mycophenolate. Mycophenolate, as used herein, can be included the methods of treatment provided herein to suppress the immune system and inhibit the development of Graft versus Host disease in the recipient. Brand names of mycophenolate can include CellCept®, Mycophenolate Mofetil Accord, Mycophenolate Mofetil Sandoz, Myfenax, Myfortic, Mycophenolate Mofetil Actavis, Mycophenolic Acid Accord, Mycophenolate Mofetil 2care4, Mycophenolate Mofetil EQL, Mycophenolate Mofetil Orifarm, and Myfortic® mycophenolic acid. The postoperative treatment regimen can include a constant course followed by a tapering course of mycophenolate administration to the recipient.

In certain embodiments, the postoperative treatment regimen provided herein comprises administering standard-of-care antimetabolite (e.g. mycophenolate) therapy to a transplant recipient. In specific embodiments, standard-of-care antimetabolite (e.g. mycophenolate) therapy is first administered to a recipient within 24 hours of reperfusion of the transplanted liver. In certain embodiments, standard-of-care antimetabolite (e.g. mycophenolate) therapy can be administered on the day of the transplant, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, 16 days after, 17 days after, 18 days after, 19 days after, 20 days after, 21 days after, 22 days after, 23 days after, 24 days after, 25 days after, 26 days after, 27 days after, 28 days after, 29 days after, 30 days after, 1 month after, 2 months after, 3 months after, 4 months after, 5 months after, 6 months after, 7 months after, 8 months after, 9 months after, 10 months after, 11 months after, 12 months after, 13 months after, 14 months after, 15 months after, 16 months after, 17 months after, or 18 months after the transplant surgery. In some embodiments, standard-of-care antimetabolite (e.g. mycophenolate) therapy is administered on the day of the transplant. In some embodiments, standard-of-care antimetabolite (e.g. mycophenolate) therapy is administered 1 day after the transplant surgery. In some embodiments, standard-of-care antimetabolite (e.g. mycophenolate) therapy is administered 2 days after the transplant surgery. In some embodiments, standard-of-care antimetabolite (e.g. mycophenolate) therapy is administered 3 days after the transplant surgery. In some embodiments, standard-of-care antimetabolite (e.g. mycophenolate) therapy is administered 4 days after the transplant surgery. In some embodiments, standard-of-care antimetabolite (e.g. mycophenolate) therapy is administered 5 days after the transplant surgery. In some embodiments, standard-of-care antimetabolite (e.g. mycophenolate) therapy is administered 6 days after the transplant surgery. In some embodiments, standard-of-care antimetabolite (e.g. mycophenolate) therapy is administered 7 days after the transplant surgery. In some embodiments, standard-of-care antimetabolite (e.g. mycophenolate) therapy is administered 8 days after the transplant surgery. In some embodiments, standard-of-care antimetabolite (e.g. mycophenolate) therapy is administered 9 days after the transplant surgery. In some embodiments, standard-of-care antimetabolite (e.g. mycophenolate) therapy is administered 10 days after the transplant surgery. In some embodiments, standard-of-care antimetabolite (e.g. mycophenolate) therapy is administered 11 days after the transplant surgery. In some embodiments, standard-of-care antimetabolite (e.g. mycophenolate) therapy is administered 12 days after the transplant surgery. In some embodiments, standard-of-care antimetabolite (e.g. mycophenolate) therapy is administered 13 days after the transplant surgery. In some embodiments, standard-of-care antimetabolite (e.g. mycophenolate) therapy is administered 14 days after the transplant surgery. In some embodiments, standard-of-care antimetabolite (e.g. mycophenolate) therapy is administered 15 days after the transplant surgery. In some embodiments, standard-of-care antimetabolite (e.g. mycophenolate) therapy is administered 16 days after the transplant surgery. In some embodiments, standard-of-care antimetabolite (e.g. mycophenolate) therapy is administered 17 days after the transplant surgery. In some embodiments, standard-of-care antimetabolite (e.g. mycophenolate) therapy is administered 18 days after the transplant surgery. In some embodiments, standard-of-care antimetabolite (e.g. mycophenolate) therapy is administered 19 days after the transplant surgery. In some embodiments, standard-of-care antimetabolite (e.g. mycophenolate) therapy is administered 20 days after the transplant surgery. In some embodiments, standard-of-care antimetabolite (e.g. mycophenolate) therapy is administered 21 days after the transplant surgery. In some embodiments, standard-of-care antimetabolite (e.g. mycophenolate) therapy is administered 22 days after the transplant surgery. In some embodiments, standard-of-care antimetabolite (e.g. mycophenolate) therapy is administered 23 days after the transplant surgery. In some embodiments, standard-of-care antimetabolite (e.g. mycophenolate) therapy is administered 24 days after the transplant surgery. In some embodiments, standard-of-care antimetabolite (e.g. mycophenolate) therapy is administered 25 days after the transplant surgery. In some embodiments, standard-of-care antimetabolite (e.g. mycophenolate) therapy is administered 26 days after the transplant surgery. In some embodiments, standard-of-care antimetabolite (e.g. mycophenolate) therapy is administered 27 days after the transplant surgery. In some embodiments, standard-of-care antimetabolite (e.g. mycophenolate) therapy is administered 28 days after the transplant surgery. In some embodiments, standard-of-care antimetabolite (e.g. mycophenolate) therapy is administered 29 days after the transplant surgery. In some embodiments, standard-of-care antimetabolite (e.g. mycophenolate) therapy is administered 30 days after. In some embodiments, standard-of-care antimetabolite (e.g. mycophenolate) therapy is administered 1 month after the transplant surgery. In some embodiments, standard-of-care antimetabolite (e.g. mycophenolate) therapy is administered 2 months after the transplant surgery. In some embodiments, standard-of-care antimetabolite (e.g. mycophenolate) therapy is administered 3 months after the transplant surgery. In some embodiments, standard-of-care antimetabolite (e.g. mycophenolate) therapy is administered 4 months after the transplant surgery. In some embodiments, standard-of-care antimetabolite (e.g. mycophenolate) therapy is administered 5 months after the transplant surgery. In some embodiments, standard-of-care antimetabolite (e.g. mycophenolate) therapy is administered 6 months after the transplant surgery. In some embodiments, standard-of-care antimetabolite (e.g. mycophenolate) therapy is administered 7 months after the transplant surgery. In some embodiments, standard-of-care antimetabolite (e.g. mycophenolate) therapy is administered 8 months after the transplant surgery. In some embodiments, standard-of-care antimetabolite (e.g. mycophenolate) therapy is administered 9 months after the transplant surgery. In some embodiments, standard-of-care antimetabolite (e.g. mycophenolate) therapy is administered 10 months after the transplant surgery. In some embodiments, standard-of-care antimetabolite (e.g. mycophenolate) therapy is administered 11 months after the transplant surgery. In some embodiments, standard-of-care antimetabolite (e.g. mycophenolate) therapy is administered 12 months after the transplant surgery. In some embodiments, standard-of-care antimetabolite (e.g. mycophenolate) therapy is administered 13 months after the transplant surgery. In some embodiments, standard-of-care antimetabolite (e.g. mycophenolate) therapy is administered 14 months after the transplant surgery. In some embodiments, standard-of-care antimetabolite (e.g. mycophenolate) therapy is administered 15 months after the transplant surgery. In some embodiments, standard-of-care antimetabolite (e.g. mycophenolate) therapy is administered 16 months after the transplant surgery. In some embodiments, standard-of-care antimetabolite (e.g. mycophenolate) therapy is administered 17 months after the transplant surgery. In some embodiments, standard-of-care antimetabolite (e.g. mycophenolate) therapy is administered 18 months after the transplant surgery.

In certain embodiments, a single dose amount of standard-of-care antimetabolite (e.g. mycophenolate) therapy can be administered. In certain embodiments, multiple dose amounts of standard-of-care antimetabolite (e.g. mycophenolate) therapy can be administered. In certain embodiments, a constant dose of standard-of-care antimetabolite (e.g. mycophenolate) therapy can be administered. In certain embodiments, a tapering course of standard-of-care antimetabolite (e.g. mycophenolate) therapy can be administered. In certain embodiments, a constant dose of standard-of-care antimetabolite (e.g. mycophenolate) therapy followed by a tapering course of standard-of-care antimetabolite (e.g. mycophenolate) therapy can be administered. In certain embodiments, standard-of-care antimetabolite (e.g. mycophenolate) therapy is administered twice a day.

In certain embodiments, standard-of-care antimetabolite (e.g. mycophenolate) therapy can be administered to a transplant recipient at a total daily dose of 100 mg/dose, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 100-1000 mg, 200-1200 mg, 300-1300 mg, 400-1400 mg, 500-1500 mg, 600-1600 mg, 700-1700 mg, 800-1800 mg, 900-1900 mg, 1000-2000 mg, 1100-2100 mg, 1200-2200 mg, 1300-2300 mg, 1400-2400 mg, or 1500-2500 mg. In some embodiments, standard-of-care antimetabolite (e.g. mycophenolate) therapy can be administered to a transplant recipient at a total daily dose of 100 mg/dose. In some embodiments, standard-of-care antimetabolite (e.g. mycophenolate) therapy can be administered to a transplant recipient at a total daily dose of 200 mg. In some embodiments, standard-of-care antimetabolite (e.g. mycophenolate) therapy can be administered to a transplant recipient at a total daily dose of 300 mg. In some embodiments, standard-of-care antimetabolite (e.g. mycophenolate) therapy can be administered to a transplant recipient at a total daily dose of 400 mg. In some embodiments, standard-of-care antimetabolite (e.g. mycophenolate) therapy can be administered to a transplant recipient at a total daily dose of 500 mg. In some embodiments, standard-of-care antimetabolite (e.g. mycophenolate) therapy can be administered to a transplant recipient at a total daily dose of 600 mg. In some embodiments, standard-of-care antimetabolite (e.g. mycophenolate) therapy can be administered to a transplant recipient at a total daily dose of 700 mg. In some embodiments, standard-of-care antimetabolite (e.g. mycophenolate) therapy can be administered to a transplant recipient at a total daily dose of 800 mg. In some embodiments, standard-of-care antimetabolite (e.g. mycophenolate) therapy can be administered to a transplant recipient at a total daily dose of 900 mg. In some embodiments, standard-of-care antimetabolite (e.g. mycophenolate) therapy can be administered to a transplant recipient at a total daily dose of 1000 mg. In some embodiments, standard-of-care antimetabolite (e.g. mycophenolate) therapy can be administered to a transplant recipient at a total daily dose of 1100 mg. In some embodiments, standard-of-care antimetabolite (e.g. mycophenolate) therapy can be administered to a transplant recipient at a total daily dose of 1200 mg. In some embodiments, standard-of-care antimetabolite (e.g. mycophenolate) therapy can be administered to a transplant recipient at a total daily dose of 1300 mg. In some embodiments, standard-of-care antimetabolite (e.g. mycophenolate) therapy can be administered to a transplant recipient at a total daily dose of 1400 mg. In some embodiments, standard-of-care antimetabolite (e.g. mycophenolate) therapy can be administered to a transplant recipient at a total daily dose of 1500 mg. In some embodiments, standard-of-care antimetabolite (e.g. mycophenolate) therapy can be administered to a transplant recipient at a total daily dose of 1600 mg. In some embodiments, standard-of-care antimetabolite (e.g. mycophenolate) therapy can be administered to a transplant recipient at a total daily dose of 1700 mg. In some embodiments, standard-of-care antimetabolite (e.g. mycophenolate) therapy can be administered to a transplant recipient at a total daily dose of 1800 mg. In some embodiments, standard-of-care antimetabolite (e.g. mycophenolate) therapy can be administered to a transplant recipient at a total daily dose of 1900 mg. In some embodiments, standard-of-care antimetabolite (e.g. mycophenolate) therapy can be administered to a transplant recipient at a total daily dose of 2000 mg. In some embodiments, standard-of-care antimetabolite (e.g. mycophenolate) therapy can be administered to a transplant recipient at a total daily dose of 2100 mg. In some embodiments, standard-of-care antimetabolite (e.g. mycophenolate) therapy can be administered to a transplant recipient at a total daily dose of 2200 mg. In some embodiments, standard-of-care antimetabolite (e.g. mycophenolate) therapy can be administered to a transplant recipient at a total daily dose of 2300 mg. In some embodiments, standard-of-care antimetabolite (e.g. mycophenolate) therapy can be administered to a transplant recipient at a total daily dose of 2400 mg. In some embodiments, standard-of-care antimetabolite (e.g. mycophenolate) therapy can be administered to a transplant recipient at a total daily dose of 2500 mg. In some embodiments, standard-of-care antimetabolite (e.g. mycophenolate) therapy can be administered to a transplant recipient at a total daily dose of 100-1000 mg. In some embodiments, standard-of-care antimetabolite (e.g. mycophenolate) therapy can be administered to a transplant recipient at a total daily dose of 200-1200 mg. In some embodiments, standard-of-care antimetabolite (e.g. mycophenolate) therapy can be administered to a transplant recipient at a total daily dose of 300-1300 mg. In some embodiments, standard-of-care antimetabolite (e.g. mycophenolate) therapy can be administered to a transplant recipient at a total daily dose of 400-1400 mg. In some embodiments, standard-of-care antimetabolite (e.g. mycophenolate) therapy can be administered to a transplant recipient at a total daily dose of 500-1500 mg. In some embodiments, standard-of-care antimetabolite (e.g. mycophenolate) therapy can be administered to a transplant recipient at a total daily dose of 600-1600 mg. In some embodiments, standard-of-care antimetabolite (e.g. mycophenolate) therapy can be administered to a transplant recipient at a total daily dose of 700-1700 mg. In some embodiments, standard-of-care antimetabolite (e.g. mycophenolate) therapy can be administered to a transplant recipient at a total daily dose of 800-1800 mg. In some embodiments, standard-of-care antimetabolite (e.g. mycophenolate) therapy can be administered to a transplant recipient at a total daily dose of 900-1900 mg. In some embodiments, standard-of-care antimetabolite (e.g. mycophenolate) therapy can be administered to a transplant recipient at a total daily dose of 1000-2000 mg. In some embodiments, standard-of-care antimetabolite (e.g. mycophenolate) therapy can be administered to a transplant recipient at a total daily dose of 1100-2100 mg. In some embodiments, standard-of-care antimetabolite (e.g. mycophenolate) therapy can be administered to a transplant recipient at a total daily dose of 1200-2200 mg. In some embodiments, standard-of-care antimetabolite (e.g. mycophenolate) therapy can be administered to a transplant recipient at a total daily dose of 1300-2300 mg. In some embodiments, standard-of-care antimetabolite (e.g. mycophenolate) therapy can be administered to a transplant recipient at a total daily dose of 1400-2400 mg. In some embodiments, standard-of-care antimetabolite (e.g. mycophenolate) therapy can be administered to a transplant recipient at a total daily dose of 1500-2500 mg. A total daily dose may be administered in 1, 2, 3, 4 or more doses per day. In some embodiments, a total daily dose may be administered in 1 dose per day. In some embodiments, a total daily dose may be administered in 2 doses per day. In some embodiments, a total daily dose may be administered in 3 doses per day. In some embodiments, a total daily dose may be administered in 4 doses per day.

In a specific embodiment, standard-of-care antimetabolite (e.g. mycophenolate) therapy is administered to a transplant recipient at a dose range of 500-1500 mg/day twice per day. In specific embodiments, standard-of-care antimetabolite (e.g. mycophenolate) therapy is administered to a transplant recipient at a dose range of 250-750 mg per dose twice a day. In particular embodiments, myfortic is administered to a transplant recipient at a total daily dose of about 1000 mg, about 1100 mg, about 1200 mg, about 1300 mg, about 1400 mg, about 1500 mg, about 1600 mg, about 1700 mg, about 1800 mg, about 1900 mg, or about 2000 mg. In some embodiments, myfortic is administered to a transplant recipient at a total daily dose of about 1000 mg. In some embodiments, myfortic is administered to a transplant recipient at a total daily dose of about 1100 mg. In some embodiments, myfortic is administered to a transplant recipient at a total daily dose of about 1200 mg. In some embodiments, myfortic is administered to a transplant recipient at a total daily dose of about 1300 mg. In some embodiments, myfortic is administered to a transplant recipient at a total daily dose of about 1400 mg. In some embodiments, myfortic is administered to a transplant recipient at a total daily dose of about 1500 mg. In some embodiments, myfortic is administered to a transplant recipient at a total daily dose of about 1600 mg. In some embodiments, myfortic is administered to a transplant recipient at a total daily dose of about 1700 mg. In some embodiments, myfortic is administered to a transplant recipient at a total daily dose of about 1800 mg. In some embodiments, myfortic is administered to a transplant recipient at a total daily dose of about 1900 mg. In some embodiments, myfortic is administered to a transplant recipient at a total daily dose of about 2000 mg.

In certain embodiments, standard-of-care antimetabolite (e.g. mycophenolate) therapy can be administered to a transplant recipient at a constant dose. The duration of time a constant dose is administered can be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days after the transplant. In some embodiments, standard-of-care antimetabolite (e.g. mycophenolate) therapy is administered to a transplant recipient at a constant dose for 1 day after the transplant. In some embodiments, standard-of-care antimetabolite (e.g. mycophenolate) therapy is administered to a transplant recipient at a constant dose for 2 days after the transplant. In some embodiments, standard-of-care antimetabolite (e.g. mycophenolate) therapy is administered to a transplant recipient at a constant dose for 3 days after the transplant. In some embodiments, standard-of-care antimetabolite (e.g. mycophenolate) therapy is administered to a transplant recipient at a constant dose for 4 days after the transplant. In some embodiments, standard-of-care antimetabolite (e.g. mycophenolate) therapy is administered to a transplant recipient at a constant dose for 5 days after the transplant. In some embodiments, standard-of-care antimetabolite (e.g. mycophenolate) therapy is administered to a transplant recipient at a constant dose for 6 days after the transplant. In some embodiments, standard-of-care antimetabolite (e.g. mycophenolate) therapy is administered to a transplant recipient at a constant dose for 7 days after the transplant. In some embodiments, standard-of-care antimetabolite (e.g. mycophenolate) therapy is administered to a transplant recipient at a constant dose for 8 days after the transplant. In some embodiments, standard-of-care antimetabolite (e.g. mycophenolate) therapy is administered to a transplant recipient at a constant dose for 9 days after the transplant. In some embodiments, standard-of-care antimetabolite (e.g. mycophenolate) therapy is administered to a transplant recipient at a constant dose for 10 days after the transplant. In some embodiments, standard-of-care antimetabolite (e.g. mycophenolate) therapy is administered to a transplant recipient at a constant dose for 11 days after the transplant. In some embodiments, standard-of-care antimetabolite (e.g. mycophenolate) therapy is administered to a transplant recipient at a constant dose for 12 days after the transplant. In some embodiments, standard-of-care antimetabolite (e.g. mycophenolate) therapy is administered to a transplant recipient at a constant dose for 13 days after the transplant. In some embodiments, standard-of-care antimetabolite (e.g. mycophenolate) therapy is administered to a transplant recipient at a constant dose for 14 days after the transplant. In some embodiments, standard-of-care antimetabolite (e.g. mycophenolate) therapy is administered to a transplant recipient at a constant dose for 15 days after the transplant. In some embodiments, standard-of-care antimetabolite (e.g. mycophenolate) therapy is administered to a transplant recipient at a constant dose for 16 days after the transplant. In some embodiments, standard-of-care antimetabolite (e.g. mycophenolate) therapy is administered to a transplant recipient at a constant dose for 17 days after the transplant. In some embodiments, standard-of-care antimetabolite (e.g. mycophenolate) therapy is administered to a transplant recipient at a constant dose for 18 days after the transplant. In some embodiments, standard-of-care antimetabolite (e.g. mycophenolate) therapy is administered to a transplant recipient at a constant dose for 19 days after the transplant. In some embodiments, standard-of-care antimetabolite (e.g. mycophenolate) therapy is administered to a transplant recipient at a constant dose for 20 days after the transplant. In some embodiments, standard-of-care antimetabolite (e.g. mycophenolate) therapy is administered to a transplant recipient at a constant dose for 21 days after the transplant. In some embodiments, standard-of-care antimetabolite (e.g. mycophenolate) therapy is administered to a transplant recipient at a constant dose for 22 days after the transplant. In some embodiments, standard-of-care antimetabolite (e.g. mycophenolate) therapy is administered to a transplant recipient at a constant dose for 23 days after the transplant. In some embodiments, standard-of-care antimetabolite (e.g. mycophenolate) therapy is administered to a transplant recipient at a constant dose for 24 days after the transplant. In some embodiments, standard-of-care antimetabolite (e.g. mycophenolate) therapy is administered to a transplant recipient at a constant dose for 25 days after the transplant. In some embodiments, standard-of-care antimetabolite (e.g. mycophenolate) therapy is administered to a transplant recipient at a constant dose for 26 days after the transplant. In some embodiments, standard-of-care antimetabolite (e.g. mycophenolate) therapy is administered to a transplant recipient at a constant dose for 27 days after the transplant. In some embodiments, standard-of-care antimetabolite (e.g. mycophenolate) therapy is administered to a transplant recipient at a constant dose for 28 days after the transplant. In some embodiments, standard-of-care antimetabolite (e.g. mycophenolate) therapy is administered to a transplant recipient at a constant dose for 29 days after the transplant. In some embodiments, standard-of-care antimetabolite (e.g. mycophenolate) therapy is administered to a transplant recipient at a constant dose for 30 days after the transplant.

In certain embodiments, standard-of-care antimetabolite (e.g. mycophenolate) therapy is administered to a transplant recipient and can be tapered to discontinuation. In certain embodiments, this tapering course can take place over 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, or 6 months after the transplant. In certain embodiments, the tapering course can take place over 1 day after the transplant. In certain embodiments, the tapering course can take place over 2 days after the transplant. In certain embodiments, the tapering course can take place over 3 days after the transplant. In certain embodiments, the tapering course can take place over 4 days after the transplant. In certain embodiments, the tapering course can take place over 5 days after the transplant. In certain embodiments, the tapering course can take place over 6 days after the transplant. In certain embodiments, the tapering course can take place over 7 days after the transplant. In certain embodiments, the tapering course can take place over 8 days after the transplant. In certain embodiments, the tapering course can take place over 9 days after the transplant. In certain embodiments, the tapering course can take place over 10 days after the transplant. In certain embodiments, the tapering course can take place over 11 days after the transplant. In certain embodiments, the tapering course can take place over 12 days after the transplant. In certain embodiments, the tapering course can take place over 13 days after the transplant. In certain embodiments, the tapering course can take place over 14 days after the transplant. In certain embodiments, the tapering course can take place over 15 days after the transplant. In certain embodiments, the tapering course can take place over 16 days after the transplant. In certain embodiments, the tapering course can take place over 17 days after the transplant. In certain embodiments, the tapering course can take place over 18 days after the transplant. In certain embodiments, the tapering course can take place over 19 days after the transplant. In certain embodiments, the tapering course can take place over 20 days after the transplant. In certain embodiments, the tapering course can take place over 21 days after the transplant. In certain embodiments, the tapering course can take place over 22 days after the transplant. In certain embodiments, the tapering course can take place over 23 days after the transplant. In certain embodiments, the tapering course can take place over 24 days after the transplant. In certain embodiments, the tapering course can take place over 25 days after the transplant. In certain embodiments, the tapering course can take place over 26 days after the transplant. In certain embodiments, the tapering course can take place over 27 days after the transplant. In certain embodiments, the tapering course can take place over 28 days after the transplant. In certain embodiments, the tapering course can take place over 29 days after the transplant. In certain embodiments, the tapering course can take place over 30 days after the transplant. In certain embodiments, the tapering course can take place over 1 month after the transplant. In certain embodiments, the tapering course can take place over 2 months after the transplant. In certain embodiments, the tapering course can take place over 3 months after the transplant. In certain embodiments, the tapering course can take place over 4 months after the transplant. In certain embodiments, the tapering course can take place over 5 months after the transplant. In certain embodiments, the tapering course can take place over 6 months after the transplant.

In certain embodiments, standard-of-care antimetabolite (e.g. mycophenolate) therapy can be administered to a transplant recipient in a convenient manner known in the art including subcutaneously, intravenously, intravascularly, topically, intra-arterially, intra-cranially, intramuscularly, orally, intra-orbitally, by inhalation, transdermally, or intra-peritoneally, or through a route of administration which allows for the proper action of the standard-of-care antimetabolite (e.g. mycophenolate) therapy by the recipient. In certain embodiments, the administration of standard-of-care antimetabolite (e.g. mycophenolate) therapy can be modified as described herein. In a specific embodiment, standard-of-care antimetabolite (e.g. mycophenolate) therapy is administered intravenously. In some embodiments, standard-of-care antimetabolite (e.g. mycophenolate) therapy is administered subcutaneously. In some embodiments, standard-of-care antimetabolite (e.g. mycophenolate) therapy is administered intravascularly. In some embodiments, standard-of-care antimetabolite (e.g. mycophenolate) therapy is administered topically. In some embodiments, standard-of-care antimetabolite (e.g. mycophenolate) therapy is administered intra-arterially. In some embodiments, standard-of-care antimetabolite (e.g. mycophenolate) therapy is administered intra-cranially. In some embodiments, standard-of-care antimetabolite (e.g. mycophenolate) therapy is administered intramuscularly. In some embodiments, standard-of-care antimetabolite (e.g. mycophenolate) therapy is administered orally. In some embodiments, standard-of-care antimetabolite (e.g. mycophenolate) therapy is administered intra-orbitally. In some embodiments, standard-of-care antimetabolite (e.g. mycophenolate) therapy is administered by inhalation. In some embodiments, standard-of-care antimetabolite (e.g. mycophenolate) therapy is administered transdermally. In some embodiments, standard-of-care antimetabolite (e.g. mycophenolate) therapy is administered intra-peritoneally. In some embodiments, standard-of-care antimetabolite (e.g. mycophenolate) therapy is administered through a route of administration which allows for the proper action of the standard-of-care antimetabolite (e.g. mycophenolate) therapy by the recipient.

7.3.7 mTOR Inhibitor

In certain embodiments, the methods described herein include administering an mTOR inhibitor to the recipient. Without being bound by theory, the mTOR inhibitor can be administered to the recipient to inhibit T-cell and B-cell activation to prevent transplant rejection. In certain embodiments, an mTOR inhibitor can be used in combination with a calcineurin inhibitor (e.g., tacrolimus) or can be used instead of the calcineurin inhibitor (e.g., tacrolimus). In specific embodiments, the mTOR inhibitor described in the methods presented herein can be rapamycin or everolimus. In specific embodiments, the mTOR inhibitor described in the methods presented herein can be rapamycin. In specific embodiments, the mTOR inhibitor described in the methods presented herein can be everolimus. In some embodiments, an mTOR inhibitor may be used in place of a calcineurin inhibitor (e.g., tacrolimus) in a method provided herein. In some embodiments, an mTOR inhibitor may be used in a recipient who experience adverse events with a calcineurin inhibitor. In specific embodiments, an mTOR inhibitor may be used in a recipient who experienced tacrolimus-related adverse events.

In certain embodiments, the postoperative regimen provided herein comprises administering rapamycin to a transplant recipient after the transplant. In certain embodiments, rapamycin is administered daily. In certain embodiments, administration of rapamycin can be initiated immediately after the transplant, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, 16 days after, 17 days after, 18 days after, 19 days after, 20 days after, 21 days after, 22 days after, 23 days after, 24 days after, 25 days after, 26 days after, 27 days after, 28 days after, 29 days after, or 30 days after the transplant surgery. In certain embodiments, administration of rapamycin is initiated immediately after the transplant. In certain embodiments, administration of rapamycin is initiated 1 day after the transplant surgery. In certain embodiments, administration of rapamycin is initiated 2 days after the transplant surgery. In certain embodiments, administration of rapamycin is initiated 3 days after the transplant surgery. In certain embodiments, administration of rapamycin is initiated 4 days after the transplant surgery. In certain embodiments, administration of rapamycin is initiated 5 days after the transplant surgery. In certain embodiments, administration of rapamycin is initiated 6 days after the transplant surgery. In certain embodiments, administration of rapamycin is initiated 7 days after the transplant surgery. In certain embodiments, administration of rapamycin is initiated 8 days after the transplant surgery. In certain embodiments, administration of rapamycin is initiated 9 days after the transplant surgery. In certain embodiments, administration of rapamycin is initiated 10 days after the transplant surgery. In certain embodiments, administration of rapamycin is initiated 11 days after the transplant surgery. In certain embodiments, administration of rapamycin is initiated 12 days after the transplant surgery. In certain embodiments, administration of rapamycin is initiated 13 days after the transplant surgery. In certain embodiments, administration of rapamycin is initiated 14 days after the transplant surgery. In certain embodiments, administration of rapamycin is initiated 15 days after the transplant surgery. In certain embodiments, administration of rapamycin is initiated 16 days after the transplant surgery. In certain embodiments, administration of rapamycin is initiated 17 days after the transplant surgery. In certain embodiments, administration of rapamycin is initiated 18 days after the transplant surgery. In certain embodiments, administration of rapamycin is initiated 19 days after the transplant surgery. In certain embodiments, administration of rapamycin is initiated 20 days after the transplant surgery. In certain embodiments, administration of rapamycin is initiated 21 days after the transplant surgery. In certain embodiments, administration of rapamycin is initiated 22 days after the transplant surgery. In certain embodiments, administration of rapamycin is initiated 23 days after the transplant surgery. In certain embodiments, administration of rapamycin is initiated 24 days after the transplant surgery. In certain embodiments, administration of rapamycin is initiated 25 days after the transplant surgery. In certain embodiments, administration of rapamycin is initiated 26 days after the transplant surgery. In certain embodiments, administration of rapamycin is initiated 27 days after the transplant surgery. In certain embodiments, administration of rapamycin is initiated 28 days after the transplant surgery. In certain embodiments, administration of rapamycin is initiated 29 days after the transplant surgery. In certain embodiments, administration of rapamycin is initiated 30 days after the transplant surgery.

In certain embodiments, rapamycin is administered for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, or at least 12 months after the transplant. In some embodiments, rapamycin is administered for at least 1 day after the transplant. In some embodiments, rapamycin is administered for at least 2 days after the transplant. In some embodiments, rapamycin is administered for at least 3 days after the transplant. In some embodiments, rapamycin is administered for at least 4 days after the transplant. In some embodiments, rapamycin is administered for at least 5 days after the transplant. In some embodiments, rapamycin is administered for at least 6 days after the transplant. In some embodiments, rapamycin is administered for at least 7 days after the transplant. In some embodiments, rapamycin is administered for at least 8 days after the transplant. In some embodiments, rapamycin is administered for at least 9 days after the transplant. In some embodiments, rapamycin is administered for at least 10 days after the transplant. In some embodiments, rapamycin is administered for at least 11 days after the transplant. In some embodiments, rapamycin is administered for at least 12 days after the transplant. In some embodiments, rapamycin is administered for at least 13 days after the transplant. In some embodiments, rapamycin is administered for at least 14 days after the transplant. In some embodiments, rapamycin is administered for at least 15 days after the transplant. In some embodiments, rapamycin is administered for at least 16 days after the transplant. In some embodiments, rapamycin is administered for at least 17 days after the transplant. In some embodiments, rapamycin is administered for at least 18 days after the transplant. In some embodiments, rapamycin is administered for at least 19 days after the transplant. In some embodiments, rapamycin is administered for at least 20 days after the transplant. In some embodiments, rapamycin is administered for at least 21 days after the transplant. In some embodiments, rapamycin is administered for at least 22 days after the transplant. In some embodiments, rapamycin is administered for at least 23 days after the transplant. In some embodiments, rapamycin is administered for at least 24 days after the transplant. In some embodiments, rapamycin is administered for at least 25 days after the transplant. In some embodiments, rapamycin is administered for at least 26 days after the transplant. In some embodiments, rapamycin is administered for at least 27 days after the transplant. In some embodiments, rapamycin is administered for at least 28 days after the transplant. In some embodiments, rapamycin is administered for at least 29 days after the transplant. In some embodiments, rapamycin is administered for at least 30 days after the transplant. In some embodiments, rapamycin is administered for at least 1 month after the transplant. In some embodiments, rapamycin is administered for at least 2 months after the transplant. In some embodiments, rapamycin is administered for at least 3 months after the transplant. In some embodiments, rapamycin is administered for at least 4 months after the transplant. In some embodiments, rapamycin is administered for at least 5 months after the transplant. In some embodiments, rapamycin is administered for at least 6 months after the transplant. In some embodiments, rapamycin is administered for at least 7 months after the transplant. In some embodiments, rapamycin is administered for at least 8 months after the transplant. In some embodiments, rapamycin is administered for at least 9 months after the transplant. In some embodiments, rapamycin is administered for at least 10 months after the transplant. In some embodiments, rapamycin is administered for at least 11 months after the transplant. In some embodiments, rapamycin is administered for at least or at least 12 months after the transplant.

In certain embodiments, rapamycin is administered in 1, 2, 3, 4, or 5 daily doses. In some embodiments, rapamycin is administered in 1 daily doses. In some embodiments, rapamycin is administered in 2 daily doses. In some embodiments, rapamycin is administered in 3 daily doses. In some embodiments, rapamycin is administered in 4 daily doses. In some embodiments, rapamycin is administered in 5 daily doses.

In certain embodiments, the first dose of rapamycin is a loading dose and can be higher than the subsequent daily doses. In certain embodiments, the first dose can be 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 1-4 mg/kg, 2-6 mg/kg, 4-8 mg/kg, 6-10 mg/kg, or 8-12 mg/kg. In some embodiments, the first dose of rapamycin is 1-3 mg/kg. In some embodiments, the first dose of rapamycin is 1.5-2.5 mg/kg. In some embodiments, the first dose of rapamycin is 0.5 mg/kg. In some embodiments, the first dose of rapamycin is 1 mg/kg. In some embodiments, the first dose of rapamycin is 1.5 mg/kg. In some embodiments, the first dose of rapamycin is 2 mg/kg. In some embodiments, the first dose of rapamycin is 2.5 mg/kg. In some embodiments, the first dose of rapamycin is 3 mg/kg. In some embodiments, the first dose of rapamycin is 3.5 mg/kg. In some embodiments, the first dose of rapamycin is 4 mg/kg. In some embodiments, the first dose of rapamycin is. In some embodiments, the first dose of rapamycin is 5 mg/kg. In some embodiments, the first dose of rapamycin is 6 mg/kg. In some embodiments, the first dose of rapamycin is 7 mg/kg. In some embodiments, the first dose of rapamycin is 8 mg/kg. In some embodiments, the first dose of rapamycin is 9 mg/kg. In some embodiments, the first dose of rapamycin is 10 mg/kg. In some embodiments, the first dose of rapamycin is 11 mg/kg. In some embodiments, the first dose of rapamycin is 12 mg/kg. In some embodiments, the first dose of rapamycin is 1-4 mg/kg. In some embodiments, the first dose of rapamycin is 2-6 mg/kg. In some embodiments, the first dose of rapamycin is 4-8 mg/kg. In some embodiments, the first dose of rapamycin is 6-10 mg/kg. In some embodiments, the first dose of rapamycin is or 8-12 mg/kg. In a specific embodiment, the rapamycin is administered at an initial dose of 6 mg/kg and followed by a daily dose of 2 mg/kg. In a specific embodiment, rapamycin is administered orally.

In certain embodiments, rapamycin can be administered postoperatively to a recipient at a sufficient dose amount to obtain the target whole blood levels of 1-5 ng/ml, 2-10 ng/ml, 4-12 ng/ml, 6-14 ng/ml, 8-16 ng/ml, 10-18 ng/ml, 12-20 ng/ml, 14-22 ng/ml, or 16-24 ng/ml. In some embodiments, the target whole blood levels is 1-5 ng/ml. In some embodiments, the target whole blood levels is 2-10 ng/ml. In some embodiments, the target whole blood levels is 4-12 ng/ml. In some embodiments, the target whole blood levels is 6-14 ng/ml. In some embodiments, the target whole blood levels is 8-16 ng/ml. In some embodiments, the target whole blood levels is 10-18 ng/ml. In some embodiments, the target whole blood levels is 12-20 ng/ml. In some embodiments, the target whole blood levels is 14-22 ng/ml. In some embodiments, the target whole blood levels is 16-24 ng/ml.

In certain embodiments, rapamycin can be administered to a transplant recipient in a convenient manner known in the art including subcutaneously, intravenously, intravascularly, topically, intra-arterially, intra-cranially, intramuscularly, orally, intra-orbitally, by inhalation, transdermally, intra-peritoneally, or through a route of administration which allows for the appropriate action of rapamycin to occur in the recipient. In a specific embodiment, rapamycin is administered intravenously. In some embodiments, rapamycin is administered subcutaneously. In some embodiments, rapamycin is administered intravascularly. In some embodiments, rapamycin is administered topically. In some embodiments, rapamycin is administered intra-arterially. In some embodiments, rapamycin is administered intra-cranially. In some embodiments, rapamycin is administered intramuscularly. In some embodiments, rapamycin is administered orally. In some embodiments, rapamycin is administered intra-orbitally. In some embodiments, rapamycin is administered by inhalation. In some embodiments, rapamycin is administered transdermally. In some embodiments, rapamycin is administered intra-peritoneally. In some embodiments, rapamycin is administered through a route of administration which allows for the depletion of T-cells in the recipient.

In certain embodiments, the mTOR inhibitor can be everolimus. In certain embodiments, everolimus is administered twice daily. In certain embodiments, administration of everolimus can be initiated immediately after the transplant, 1 day after, 2 days after, 3 days after, 4 days after, 5 days after, 6 days after, 7 days after, 8 days after, 9 days after, 10 days after, 11 days after, 12 days after, 13 days after, 14 days after, 15 days after, 16 days after, 17 days after, 18 days after, 19 days after, 20 days after, 21 days after, 22 days after, 23 days after, 24 days after, 25 days after, 26 days after, 27 days after, 28 days after, 29 days after, or 30 days after the transplant surgery. In some embodiments, everolimus is administered immediately after transplant. In some embodiments, everolimus is administered to the recipient 1 day after transplant. In some embodiments, everolimus is administered to the recipient 2 days after transplant. In some embodiments, everolimus is administered to the recipient 3 days after transplant. In some embodiments, everolimus is administered to the recipient 4 days after transplant. In some embodiments, everolimus is administered to the recipient 5 days after transplant. In some embodiments, everolimus is administered to the recipient 6 days after transplant. In some embodiments, everolimus is administered to the recipient 7 days after transplant. In some embodiments, everolimus is administered to the recipient 8 days after transplant. In some embodiments, everolimus is administered to the recipient 9 days after transplant. In some embodiments, everolimus is administered to the recipient 10 days after transplant. In some embodiments, everolimus is administered to the recipient 11 days after transplant. In some embodiments, everolimus is administered to the recipient 12 days after transplant. In some embodiments, everolimus is administered to the recipient 14 days after transplant. In some embodiments, everolimus is administered to the recipient 15 days after transplant. In some embodiments, everolimus is administered to the recipient 16 days after transplant. In some embodiments, everolimus is administered to the recipient 17 days after transplant. In some embodiments, everolimus is administered to the recipient 18 days after transplant. In some embodiments, everolimus is administered to the recipient 19 days after transplant. In some embodiments, everolimus is administered to the recipient 20 days after transplant. In some embodiments, everolimus is administered to the recipient 21 days after transplant. In some embodiments, everolimus is administered to the recipient 22 days after transplant. In some embodiments, everolimus is administered to the recipient 23 days after transplant. In some embodiments, everolimus is administered to the recipient 24 days after transplant. In some embodiments, everolimus is administered to the recipient 25 days after transplant. In some embodiments, everolimus is administered to the recipient 26 days after transplant. In some embodiments, everolimus is administered to the recipient 27 days after transplant. In some embodiments, everolimus is administered to the recipient 28 days after transplant. In some embodiments, everolimus is administered to the recipient 29 days after transplant. In some embodiments, everolimus is administered to the recipient 30 days after transplant.

In certain embodiments, everolimus is administered for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, or at least 12 months after the transplant. In some embodiments, everolimus is administered for at least 1 day after the transplant. In some embodiments, everolimus is administered for at least 2 days after the transplant. In some embodiments, everolimus is administered for at least 3 days after the transplant. In some embodiments, everolimus is administered for at least 4 days after the transplant. In some embodiments, everolimus is administered for at least 5 days after the transplant. In some embodiments, everolimus is administered for at least 6 days after the transplant. In some embodiments, everolimus is administered for at least 7 days after the transplant. In some embodiments, everolimus is administered for at least 8 days after the transplant. In some embodiments, everolimus is administered for at least 9 days after the transplant. In some embodiments, everolimus is administered for at least 10 days after the transplant. In some embodiments, everolimus is administered for at least 11 days after the transplant. In some embodiments, everolimus is administered for at least 12 days after the transplant. In some embodiments, everolimus is administered for at least 13 days after the transplant. In some embodiments, everolimus is administered for at least 14 days after the transplant. In some embodiments, everolimus is administered for at least 15 days after the transplant. In some embodiments, everolimus is administered for at least 16 days after the transplant. In some embodiments, everolimus is administered for at least 17 days after the transplant. In some embodiments, everolimus is administered for at least 18 days after the transplant. In some embodiments, everolimus is administered for at least 19 days after the transplant. In some embodiments, everolimus is administered for at least 20 days after the transplant. In some embodiments, everolimus is administered for at least 21 days after the transplant. In some embodiments, everolimus is administered for at least 22 days after the transplant. In some embodiments, everolimus is administered for at least 23 days after the transplant. In some embodiments, everolimus is administered for at least 24 days after the transplant. In some embodiments, everolimus is administered for at least 25 days after the transplant. In some embodiments, everolimus is administered for at least 26 days after the transplant. In some embodiments, everolimus is administered for at least 27 days after the transplant. In some embodiments, everolimus is administered for at least 28 days after the transplant. In some embodiments, everolimus is administered for at least 29 days after the transplant. In some embodiments, everolimus is administered for at least 30 days after the transplant. In some embodiments, everolimus is administered for at least 1 month after the transplant. In some embodiments, everolimus is administered for at least 2 months after the transplant. In some embodiments, everolimus is administered for at least 3 months after the transplant. In some embodiments, everolimus is administered for at least 4 months after the transplant. In some embodiments, everolimus is administered for at least 5 months after the transplant. In some embodiments, everolimus is administered for at least 6 months after the transplant. In some embodiments, everolimus is administered for at least 7 months after the transplant. In some embodiments, everolimus is administered for at least 8 months after the transplant. In some embodiments, everolimus is administered for at least 9 months after the transplant. In some embodiments, everolimus is administered for at least 10 months after the transplant. In some embodiments, everolimus is administered for at least 11 months after the transplant. In some embodiments, everolimus is administered for at least or at least 12 months after the transplant.

In certain embodiments, the dose range of everolimus administered can be 0.25 mg/kg/dose, 0.5 mg/kg/dose, 0.75 mg/kg/dose, 1.0 mg/kg/dose, 1.25 mg/kg/dose, 1.5 mg/kg/dose, 1.75 mg/kg/dose, 2.0 mg/kg/dose, 0.25-0.5 mg/kg/dose, 0.5-0.75 mg/kg/dose, 0.75-1.0 mg/kg/dose, 1.0-1.25 mg/kg/dose, 1.25-1.50 mg/kg/dose, 1.50-1.75 mg/kg/dose, or 1.75-2.0 mg/kg/dose. In a specific embodiment, the everolimus is administered twice daily at a dose range of 0.75-1.0 mg/kg/dose. In some embodiments, the dose of everolimus administered is 0.25 mg/kg/dose. In some embodiments, the dose of everolimus administered is 0.5 mg/kg/dose. In some embodiments, the dose of everolimus administered is 0.75 mg/kg/dose. In some embodiments, the dose of everolimus administered is 1.0 mg/kg/dose. In some embodiments, the dose of everolimus administered is 1.25 mg/kg/dose. In some embodiments, the dose of everolimus administered is 1.5 mg/kg/dose. In some embodiments, the dose of everolimus administered is 1.75 mg/kg/dose. In some embodiments, the dose of everolimus administered is 2.0 mg/kg/dose. In some embodiments, the dose of everolimus administered is 0.25-0.5 mg/kg/dose. In some embodiments, the dose of everolimus administered is 0.5-0.75 mg/kg/dose. In some embodiments, the dose of everolimus administered is 0.75-1.0 mg/kg/dose. In some embodiments, the dose of everolimus administered is 1.0-1.25 mg/kg/dose. In some embodiments, the dose of everolimus administered is 1.25-1.50 mg/kg/dose. In some embodiments, the dose of everolimus administered is 1.50-1.75 mg/kg/dose. In some embodiments, the dose of everolimus administered is 1.75-2.0 mg/kg/dose. In a specific embodiment, everolimus is administered orally.

In certain embodiments, everolimus can be administered postoperatively to a recipient at a sufficient dose amount to obtain the target whole blood levels of 0.1-5 ng/ml, 1-6 ng/ml, 2-7 ng/ml, 3-8 ng/ml, 4-9 ng/ml, 5-10 ng/ml, 6-11 ng/ml, 7-12 ng/ml, or 8-13 ng/ml. In some embodiments, the target whole blood levels is 1-5 ng/ml. In some embodiments, the target whole blood levels is 1-6 ng/ml. In some embodiments, the target whole blood levels is 2-7 ng/ml. In some embodiments, the target whole blood levels is 3-8 ng/ml. In some embodiments, the target whole blood levels is 4-9 ng/ml. In some embodiments, the target whole blood levels is 5-10 ng/ml. In some embodiments, the target whole blood levels is 6-11 ng/ml. In some embodiments, the target whole blood levels is 7-12 ng/ml. In some embodiments, the target whole blood levels is 8-13 ng/ml.

In certain embodiments, the mTOR inhibitor can be temsirolimus, everolimus, sirolimus (rapamycin), ridaforolimus, non-rapamycin analog mTOR inhibiting compounds including, but not limited to, LY294002, wortmannin, quercetin, myricentin, staurosporine, or ATP competitive inhibitors. In some embodiments, the mTOR inhibitor comprises temsirolimus. In some embodiments, the mTOR inhibitor comprises everolimus. In some embodiments, the mTOR inhibitor comprises sirolimus (rapamycin). In some embodiments, the mTOR inhibitor comprises ridaforolimus. In some embodiments, the mTOR inhibitor comprises non-rapamycin analog mTOR inhibiting compounds including. In some embodiments, the mTOR inhibitor comprises but not limited to. In some embodiments, the mTOR inhibitor comprises LY294002. In some embodiments, the mTOR inhibitor comprises wortmannin. In some embodiments, the mTOR inhibitor comprises quercetin. In some embodiments, the mTOR inhibitor comprises myricentin. In some embodiments, the mTOR inhibitor comprises staurosporine. In some embodiments, the mTOR inhibitor comprises a ATP competitive inhibitor.

### 7.4 Outcome Assessments

Exemplary methods of assessing the outcome of a method described herein are described in sections 8.2 and 8.3 below.

In certain embodiments, the efficacy of a method of treatment described herein can be assessed by determining the survival of the transplanted graft (e.g., liver).

In certain embodiments, a biopsy of the transplanted graft (e.g., liver) can be performed to examine the health of the transplanted organ (e.g., liver) and determine induction of tolerance or evidence of graft rejection. Tissue biopsies can be examined using routine light microscopy, immunofluorescence, and electron microscopy.

In certain embodiments, the efficacy of a method of treatment described herein can be determined by detecting treated biopsy-proven acute rejection (tBPAR) in a recipient. In specific embodiments, no tBPAR is detected in a recipient at 10 months, 12 months, 15 months, 18 months, 20 months, 25 months, 30 months, 35 months, 40 months, 45 months, 50 months, 55 months, or 60 months post-transplant. In some embodiments, no tBPAR is detected in a recipient at 10 months. In some embodiments, no tBPAR is detected in a recipient at 12 months. In some embodiments, no tBPAR is detected in a recipient at 15 months. In some embodiments, no tBPAR is detected in a recipient at 18 months. In some embodiments, no tBPAR is detected in a recipient at 20 months. In some embodiments, no tBPAR is detected in a recipient at 25 months. In some embodiments, no tBPAR is detected in a recipient at 30 months. In some embodiments, no tBPAR is detected in a recipient at 35 months. In some embodiments, no tBPAR is detected in a recipient at 40 months. In some embodiments, no tBPAR is detected in a recipient at 45 months. In some embodiments, no tBPAR is detected in a recipient at 50 months. In some embodiments, no tBPAR is detected in a recipient at 55 months. In some embodiments, no tBPAR is detected in a recipient at 60 months post-transplant.

In certain embodiments, assessments of the outcome of the transplant surgery can include the monitoring of the function of the transplanted organ (e.g., liver) in the recipient. For example, the efficacy of a method of treatment described herein may be assessed by measuring liver function at 1 month, 2 months, 3 months, 4 months, 5 months, 6 months, 7.5 months, 9 months, 10.5 months, 12 months, 13.5 months, 15 months, 16.5 months, 18 months, 19.5 months, 21 months, 22.5 months, 24 months, 27 months, 30 months, 36 months, 42 months, 48 months, or, 54 months post-transplant, or at 1-5 months, 5-10 months, 10-15 months, 15-20 months, 20-25 months, 25-30 months, 30-35 months, 35-40 months, 40-45 months, 45-50 months, 50-55 months, or 55-60 months post-transplant. In some embodiments, efficacy of a method of treatment described herein is assessed by measuring liver function at 1 month. In some embodiments, efficacy of a method of treatment described herein is assessed by measuring liver function at 2 months. In some embodiments, efficacy of a method of treatment described herein is assessed by measuring liver function at 3 months. In some embodiments, efficacy of a method of treatment described herein is assessed by measuring liver function at 4 months. In some embodiments, efficacy of a method of treatment described herein is assessed by measuring liver function at 5 months. In some embodiments, efficacy of a method of treatment described herein is assessed by measuring liver function at 6 months. In some embodiments, efficacy of a method of treatment described herein is assessed by measuring liver function at 7.5 months. In some embodiments, efficacy of a method of treatment described herein is assessed by measuring liver function at 9 months. In some embodiments, efficacy of a method of treatment described herein is assessed by measuring liver function at 10.5 months. In some embodiments, efficacy of a method of treatment described herein is assessed by measuring liver function at 12 months. In some embodiments, efficacy of a method of treatment described herein is assessed by measuring liver function at 13.5 months. In some embodiments, efficacy of a method of treatment described herein is assessed by measuring liver function at 15 months. In some embodiments, efficacy of a method of treatment described herein is assessed by measuring liver function at 16.5 months. In some embodiments, efficacy of a method of treatment described herein is assessed by measuring liver function at 18 months. In some embodiments, efficacy of a method of treatment described herein is assessed by measuring liver function at 19.5 months. In some embodiments, efficacy of a method of treatment described herein is assessed by measuring liver function at 21 months. In some embodiments, efficacy of a method of treatment described herein is assessed by measuring liver function at 22.5 months. In some embodiments, efficacy of a method of treatment described herein is assessed by measuring liver function at 24 months. In some embodiments, efficacy of a method of treatment described herein is assessed by measuring liver function at 27 months. In some embodiments, efficacy of a method of treatment described herein is assessed by measuring liver function at 30 months. In some embodiments, efficacy of a method of treatment described herein is assessed by measuring liver function at 36 months. In some embodiments, efficacy of a method of treatment described herein is assessed by measuring liver function at 42 months. In some embodiments, efficacy of a method of treatment described herein is assessed by measuring liver function at 48 months. In some embodiments, efficacy of a method of treatment described herein is assessed by measuring liver function at or. In some embodiments, efficacy of a method of treatment described herein is assessed by measuring liver function at 54 months post-transplant. In some embodiments, efficacy of a method of treatment described herein is assessed by measuring liver function at or at 1-5 months. In some embodiments, efficacy of a method of treatment described herein is assessed by measuring liver function at 5-10 months. In some embodiments, efficacy of a method of treatment described herein is assessed by measuring liver function at 10-15 months. In some embodiments, efficacy of a method of treatment described herein is assessed by measuring liver function at 15-20 months. In some embodiments, efficacy of a method of treatment described herein is assessed by measuring liver function at 20-25 months. In some embodiments, efficacy of a method of treatment described herein is assessed by measuring liver function at 25-30 months. In some embodiments, efficacy of a method of treatment described herein is assessed by measuring liver function at 30-35 months. In some embodiments, efficacy of a method of treatment described herein is assessed by measuring liver function at 35-40 months. In some embodiments, efficacy of a method of treatment described herein is assessed by measuring liver function at 40-45 months. In some embodiments, efficacy of a method of treatment described herein is assessed by measuring liver function at 45-50 months. In some embodiments, efficacy of a method of treatment described herein is assessed by measuring liver function at 50-55 months. In some embodiments, efficacy of a method of treatment described herein is assessed by measuring liver function at 55-60 months post-transplant.

Liver function may be determined, for example, by conducting liver function tests, such as measurements of alanine transaminase (ALT), aspartate transaminase (AST), alkaline phosphatase (ALP), gamma-glutamyl transferase (GGT), serum bilirubin, prothrombin time (PT), the international normalized ratio (INR) and/or albumin. In some embodiments, liver function is determined by measurement of alanine transaminase (ALT). In some embodiments, liver function is determined by measurement of aspartate transaminase (AST). In some embodiments, liver function is determined by measurement of alkaline phosphatase (ALP). In some embodiments, liver function is determined by measurement of gamma-glutamyl transferase (GGT). In some embodiments, liver function is determined by measurement of serum bilirubin. In some embodiments, liver function is determined by measurement of prothrombin time (PT). In some embodiments, liver function is determined by measurement of the international normalized ratio (INR) and albumin. In some embodiments, liver function is determined by measurement of albumin. In some embodiments, liver function is determined by measurement of alanine transaminase (ALT), and aspartate transaminase (AST). In some embodiments, liver function is determined by measurement of alanine transaminase (ALT), aspartate transaminase (AST), alkaline phosphatase (ALP), serum bilirubin, and the international normalized ratio (INR). In some embodiments, liver function is determined by measurement of alanine transaminase (ALT), aspartate transaminase (AST), alkaline phosphatase (ALP), gamma-glutamyl transferase (GGT), serum bilirubin, prothrombin time (PT), the international normalized ratio (INR) and albumin.

In particular embodiments, a method of treatment described herein results in a recipient having normal liver function (as determined by liver function tests) for at least 10 months, at least 15 months, at least 20 months, at least 25 months, at least 30 months, at least 35 months, at least 40 months, at least 45 months, at least 50 months, at least 55 months, or at least 60 months post-transplant. In some embodiments, a method of treatment described herein results in a recipient having normal liver function (as determined by liver function tests) for at least 10 months post-transplant. In some embodiments, a method of treatment described herein results in a recipient having normal liver function (as determined by liver function tests) for at least 15 months post-transplant. In some embodiments, a method of treatment described herein results in a recipient having normal liver function (as determined by liver function tests) for at least 20 months post-transplant. In some embodiments, a method of treatment described herein results in a recipient having normal liver function (as determined by liver function tests) for at least 25 months post-transplant. In some embodiments, a method of treatment described herein results in a recipient having normal liver function (as determined by liver function tests) for at least 30 months post-transplant. In some embodiments, a method of treatment described herein results in a recipient having normal liver function (as determined by liver function tests) for at least 35 months post-transplant. In some embodiments, a method of treatment described herein results in a recipient having normal liver function (as determined by liver function tests) for at least 40 months post-transplant. In some embodiments, a method of treatment described herein results in a recipient having normal liver function (as determined by liver function tests) for at least 45 months post-transplant. In some embodiments, a method of treatment described herein results in a recipient having normal liver function (as determined by liver function tests) for at least 50 months post-transplant. In some embodiments, a method of treatment described herein results in a recipient having normal liver function (as determined by liver function tests) for at least 55 months post-transplant. In some embodiments, a method of treatment described herein results in a recipient having normal liver function (as determined by liver function tests) for at least 60 months post-transplant.

In particular embodiments, a method of treatment described herein results in a recipient having better liver function (as determined by liver function tests) for at least 10 months, at least 15 months, at least 20 months, at least 25 months, at least 30 months, at least 35 months, at least 40 months, at least 45 months, at least 50 months, at least 55 months, or at least 60 months post-transplant as compared to a patient undergoing standard of care post-liver transplant therapy. In some embodiments, a method of treatment described herein results in a recipient having better liver function (as determined by liver function tests) for at least 10 months post-transplant as compared to a patient undergoing standard of care post-liver transplant therapy. In some embodiments, a method of treatment described herein results in a recipient having better liver function (as determined by liver function tests) for at least 15 months post-transplant as compared to a patient undergoing standard of care post-liver transplant therapy. In some embodiments, a method of treatment described herein results in a recipient having better liver function (as determined by liver function tests) for at least 20 months post-transplant as compared to a patient undergoing standard of care post-liver transplant therapy. In some embodiments, a method of treatment described herein results in a recipient having better liver function (as determined by liver function tests) for at least 25 months post-transplant as compared to a patient undergoing standard of care post-liver transplant therapy. In some embodiments, a method of treatment described herein results in a recipient having better liver function (as determined by liver function tests) for at least 30 months post-transplant as compared to a patient undergoing standard of care post-liver transplant therapy. In some embodiments, a method of treatment described herein results in a recipient having better liver function (as determined by liver function tests) for at least 35 months post-transplant as compared to a patient undergoing standard of care post-liver transplant therapy. In some embodiments, a method of treatment described herein results in a recipient having better liver function (as determined by liver function tests) for at least 40 months post-transplant as compared to a patient undergoing standard of care post-liver transplant therapy. In some embodiments, a method of treatment described herein results in a recipient having better liver function (as determined by liver function tests) for at least 45 months post-transplant as compared to a patient undergoing standard of care post-liver transplant therapy. In some embodiments, a method of treatment described herein results in a recipient having better liver function (as determined by liver function tests) for at least 50 months post-transplant as compared to a patient undergoing standard of care post-liver transplant therapy. In some embodiments, a method of treatment described herein results in a recipient having better liver function (as determined by liver function tests) for at least 55 months post-transplant as compared to a patient undergoing standard of care post-liver transplant therapy. In some embodiments, a method of treatment described herein results in a recipient having better liver function (as determined by liver function tests) for at least 60 months post-transplant as compared to a patient undergoing standard of care post-liver transplant therapy.

In specific embodiments, a method of treatment described herein results in a recipient having ALT values of 5-60 U/L, 7-55 U/L, 10-55 U/L, 15-50 U/L, 20-40 U/L or 25-35 U/L for at least 10 months, at least 15 months, at least 20 months, at least 25 months, at least 30 months, at least 35 months, at least 40 months, at least 45 months, at least 50 months, at least 55 months, or at least 60 months post-transplant. In some embodiments, a method of treatment described herein results in a recipient having ALT values of 5-60 U/L, 7-55 U/L, 10-55 U/L, 15-50 U/L, 20-40 U/L or 25-35 U/L for at least 10 months post-transplant. In some embodiments, a method of treatment described herein results in a recipient having ALT values of 5-60 U/L, 7-55 U/L, 10-55 U/L, 15-50 U/L, 20-40 U/L or 25-35 U/L for at least 15 months post-transplant. In some embodiments, a method of treatment described herein results in a recipient having ALT values of 5-60 U/L, 7-55 U/L, 10-55 U/L, 15-50 U/L, 20-40 U/L or 25-35 U/L for at least 20 months post-transplant. In some embodiments, a method of treatment described herein results in a recipient having ALT values of 5-60 U/L, 7-55 U/L, 10-55 U/L, 15-50 U/L, 20-40 U/L or 25-35 U/L for at least 25 months post-transplant. In some embodiments, a method of treatment described herein results in a recipient having ALT values of 5-60 U/L, 7-55 U/L, 10-55 U/L, 15-50 U/L, 20-40 U/L or 25-35 U/L for at least 30 months post-transplant. In some embodiments, a method of treatment described herein results in a recipient having ALT values of 5-60 U/L, 7-55 U/L, 10-55 U/L, 15-50 U/L, 20-40 U/L or 25-35 U/L for at least 35 months post-transplant. In some embodiments, a method of treatment described herein results in a recipient having ALT values of 5-60 U/L, 7-55 U/L, 10-55 U/L, 15-50 U/L, 20-40 U/L or 25-35 U/L for at least 40 months post-transplant. In some embodiments, a method of treatment described herein results in a recipient having ALT values of 5-60 U/L, 7-55 U/L, 10-55 U/L, 15-50 U/L, 20-40 U/L or 25-35 U/L for at least 45 months post-transplant. In some embodiments, a method of treatment described herein results in a recipient having ALT values of 5-60 U/L, 7-55 U/L, 10-55 U/L, 15-50 U/L, 20-40 U/L or 25-35 U/L for at least 50 months post-transplant. In some embodiments, a method of treatment described herein results in a recipient having ALT values of 5-60 U/L, 7-55 U/L, 10-55 U/L, 15-50 U/L, 20-40 U/L or 25-35 U/L for at least 55 months post-transplant. In some embodiments, a method of treatment described herein results in a recipient having ALT values of 5-60 U/L, 7-55 U/L, 10-55 U/L, 15-50 U/L, 20-40 U/L or 25-35 U/L for at least 60 months post-transplant.

In specific embodiments, a method of treatment described herein results in a recipient having AST values of 5-50 U/L, 8-48 U/L, 10-45 U/L, 15-40 U/L, or 20-30 U/L for at least 10 months, at least 15 months, at least 20 months, at least 25 months, at least 30 months, at least 35 months, at least 40 months, at least 45 months, at least 50 months, at least 55 months, or at least 60 months post-transplant. In some embodiments, a method of treatment described herein results in a recipient having AST values of 5-50 U/L, 8-48 U/L, 10-45 U/L, 15-40 U/L, or 20-30 U/L for at least 10 months post-transplant. In some embodiments, a method of treatment described herein results in a recipient having AST values of 5-50 U/L, 8-48 U/L, 10-45 U/L, 15-40 U/L, or 20-30 U/L for at least 15 months post-transplant. In some embodiments, a method of treatment described herein results in a recipient having AST values of 5-50 U/L, 8-48 U/L, 10-45 U/L, 15-40 U/L, or 20-30 U/L for at least 20 months post-transplant. In some embodiments, a method of treatment described herein results in a recipient having AST values of 5-50 U/L, 8-48 U/L, 10-45 U/L, 15-40 U/L, or 20-30 U/L for at least 25 months post-transplant. In some embodiments, a method of treatment described herein results in a recipient having AST values of 5-50 U/L, 8-48 U/L, 10-45 U/L, 15-40 U/L, or 20-30 U/L for at least 30 months post-transplant. In some embodiments, a method of treatment described herein results in a recipient having AST values of 5-50 U/L, 8-48 U/L, 10-45 U/L, 15-40 U/L, or 20-30 U/L for at least 35 months post-transplant. In some embodiments, a method of treatment described herein results in a recipient having AST values of 5-50 U/L, 8-48 U/L, 10-45 U/L, 15-40 U/L, or 20-30 U/L for at least 40 months post-transplant. In some embodiments, a method of treatment described herein results in a recipient having AST values of 5-50 U/L, 8-48 U/L, 10-45 U/L, 15-40 U/L, or 20-30 U/L for at least 45 months post-transplant. In some embodiments, a method of treatment described herein results in a recipient having AST values of 5-50 U/L, 8-48 U/L, 10-45 U/L, 15-40 U/L, or 20-30 U/L for at least 50 months post-transplant. In some embodiments, a method of treatment described herein results in a recipient having AST values of 5-50 U/L, 8-48 U/L, 10-45 U/L, 15-40 U/L, or 20-30 U/L for at least 55 months post-transplant. In some embodiments, a method of treatment described herein results in a recipient having AST values of 5-50 U/L, 8-48 U/L, 10-45 U/L, 15-40 U/L, or 20-30 U/L for at least 60 months post-transplant.

In specific embodiments, a method of treatment described herein results in a recipient having ALP values of 30-150 U/L, 40-129 U/L, 50-120 U/L, 60-110 U/L, 70-100 U/L, or 80-90 U/L for at least 10 months, at least 15 months, at least 20 months, at least 25 months, at least 30 months, at least 35 months, at least 40 months, at least 45 months, at least 50 months, at least 55 months, or at least 60 months post-transplant. In some embodiments, a method of treatment described herein results in a recipient having ALP values of 30-150 U/L, 40-129 U/L, 50-120 U/L, 60-110 U/L, 70-100 U/L, or 80-90 U/L for at least 10 months post-transplant. In some embodiments, a method of treatment described herein results in a recipient having ALP values of 30-150 U/L, 40-129 U/L, 50-120 U/L, 60-110 U/L, 70-100 U/L, or 80-90 U/L for at least 15 months post-transplant. In some embodiments, a method of treatment described herein results in a recipient having ALP values of 30-150 U/L, 40-129 U/L, 50-120 U/L, 60-110 U/L, 70-100 U/L, or 80-90 U/L for at least 20 months post-transplant. In some embodiments, a method of treatment described herein results in a recipient having ALP values of 30-150 U/L, 40-129 U/L, 50-120 U/L, 60-110 U/L, 70-100 U/L, or 80-90 U/L for at least 25 months post-transplant. In some embodiments, a method of treatment described herein results in a recipient having ALP values of 30-150 U/L, 40-129 U/L, 50-120 U/L, 60-110 U/L, 70-100 U/L, or 80-90 U/L for at least 30 months post-transplant. In some embodiments, a method of treatment described herein results in a recipient having ALP values of 30-150 U/L, 40-129 U/L, 50-120 U/L, 60-110 U/L, 70-100 U/L, or 80-90 U/L for at least 35 months post-transplant. In some embodiments, a method of treatment described herein results in a recipient having ALP values of 30-150 U/L, 40-129 U/L, 50-120 U/L, 60-110 U/L, 70-100 U/L, or 80-90 U/L for at least 40 months post-transplant. In some embodiments, a method of treatment described herein results in a recipient having ALP values of 30-150 U/L, 40-129 U/L, 50-120 U/L, 60-110 U/L, 70-100 U/L, or 80-90 U/L for at least 45 months post-transplant. In some embodiments, a method of treatment described herein results in a recipient having ALP values of 30-150 U/L, 40-129 U/L, 50-120 U/L, 60-110 U/L, 70-100 U/L, or 80-90 U/L for at least 50 months post-transplant. In some embodiments, a method of treatment described herein results in a recipient having ALP values of 30-150 U/L, 40-129 U/L, 50-120 U/L, 60-110 U/L, 70-100 U/L, or 80-90 U/L for at least 55 months post-transplant. In some embodiments, a method of treatment described herein results in a recipient having ALP values of 30-150 U/L, 40-129 U/L, 50-120 U/L, 60-110 U/L, 70-100 U/L, or 80-90 U/L for at least 60 months post-transplant.

In specific embodiments, a method of treatment described herein results in a recipient having GGT values of 5-70 U/L, 8-61 U/L, 10-50 U/L, 15-45 U/L, 20-40 U/L, or 25-35 U/L for at least 10 months, at least 15 months, at least 20 months, at least 25 months, at least 30 months, at least 35 months, at least 40 months, at least 45 months, at least 50 months, at least 55 months, or at least 60 months post-transplant. In some embodiments, a method of treatment described herein results in a recipient having GGT values of 5-70 U/L, 8-61 U/L, 10-50 U/L, 15-45 U/L, 20-40 U/L, or 25-35 U/L for at least 10 months post-transplant. In some embodiments, a method of treatment described herein results in a recipient having GGT values of 5-70 U/L, 8-61 U/L, 10-50 U/L, 15-45 U/L, 20-40 U/L, or 25-35 U/L for at least 15 months post-transplant. In some embodiments, a method of treatment described herein results in a recipient having GGT values of 5-70 U/L, 8-61 U/L, 10-50 U/L, 15-45 U/L, 20-40 U/L, or 25-35 U/L for at least 20 months post-transplant. In some embodiments, a method of treatment described herein results in a recipient having GGT values of 5-70 U/L, 8-61 U/L, 10-50 U/L, 15-45 U/L, 20-40 U/L, or 25-35 U/L for at least 25 months post-transplant. In some embodiments, a method of treatment described herein results in a recipient having GGT values of 5-70 U/L, 8-61 U/L, 10-50 U/L, 15-45 U/L, 20-40 U/L, or 25-35 U/L for at least 30 months post-transplant. In some embodiments, a method of treatment described herein results in a recipient having GGT values of 5-70 U/L, 8-61 U/L, 10-50 U/L, 15-45 U/L, 20-40 U/L, or 25-35 U/L for at least 35 months post-transplant. In some embodiments, a method of treatment described herein results in a recipient having GGT values of 5-70 U/L, 8-61 U/L, 10-50 U/L, 15-45 U/L, 20-40 U/L, or 25-35 U/L for at least 40 months post-transplant. In some embodiments, a method of treatment described herein results in a recipient having GGT values of 5-70 U/L, 8-61 U/L, 10-50 U/L, 15-45 U/L, 20-40 U/L, or 25-35 U/L for at least 45 months post-transplant. In some embodiments, a method of treatment described herein results in a recipient having GGT values of 5-70 U/L, 8-61 U/L, 10-50 U/L, 15-45 U/L, 20-40 U/L, or 25-35 U/L for at least 50 months post-transplant. In some embodiments, a method of treatment described herein results in a recipient having GGT values of 5-70 U/L, 8-61 U/L, 10-50 U/L, 15-45 U/L, 20-40 U/L, or 25-35 U/L for at least 55 months post-transplant. In some embodiments, a method of treatment described herein results in a recipient having GGT values of 5-70 U/L, 8-61 U/L, 10-50 U/L, 15-45 U/L, 20-40 U/L, or 25-35 U/L for at least 60 months post-transplant.

In specific embodiments, a method of treatment described herein results in a recipient having serum bilirubin values of 0.05-2 mg/dL, 0.1-1.5 mg/dL, 0.1-1.2 mg/dL, 0.5-1 mg/dL or 0.7-1 mg/dL for at least 10 months, at least 15 months, at least 20 months, at least 25 months, at least 30 months, at least 35 months, at least 40 months, at least 45 months, at least 50 months, at least 55 months, or at least 60 months post-transplant. In some embodiments, a method of treatment described herein results in a recipient having serum bilirubin values of 0.05-2 mg/dL, 0.1-1.5 mg/dL, 0.1-1.2 mg/dL, 0.5-1 mg/dL or 0.7-1 mg/dL for at least 10 months post-transplant. In some embodiments, a method of treatment described herein results in a recipient having serum bilirubin values of 0.05-2 mg/dL, 0.1-1.5 mg/dL, 0.1-1.2 mg/dL, 0.5-1 mg/dL or 0.7-1 mg/dL for at least 15 months post-transplant. In some embodiments, a method of treatment described herein results in a recipient having serum bilirubin values of 0.05-2 mg/dL, 0.1-1.5 mg/dL, 0.1-1.2 mg/dL, 0.5-1 mg/dL or 0.7-1 mg/dL for at least 20 months post-transplant. In some embodiments, a method of treatment described herein results in a recipient having serum bilirubin values of 0.05-2 mg/dL, 0.1-1.5 mg/dL, 0.1-1.2 mg/dL, 0.5-1 mg/dL or 0.7-1 mg/dL for at least 25 months post-transplant. In some embodiments, a method of treatment described herein results in a recipient having serum bilirubin values of 0.05-2 mg/dL, 0.1-1.5 mg/dL, 0.1-1.2 mg/dL, 0.5-1 mg/dL or 0.7-1 mg/dL for at least 30 months post-transplant. In some embodiments, a method of treatment described herein results in a recipient having serum bilirubin values of 0.05-2 mg/dL, 0.1-1.5 mg/dL, 0.1-1.2 mg/dL, 0.5-1 mg/dL or 0.7-1 mg/dL for at least 35 months post-transplant. In some embodiments, a method of treatment described herein results in a recipient having serum bilirubin values of 0.05-2 mg/dL, 0.1-1.5 mg/dL, 0.1-1.2 mg/dL, 0.5-1 mg/dL or 0.7-1 mg/dL for at least 40 months post-transplant. In some embodiments, a method of treatment described herein results in a recipient having serum bilirubin values of 0.05-2 mg/dL, 0.1-1.5 mg/dL, 0.1-1.2 mg/dL, 0.5-1 mg/dL or 0.7-1 mg/dL for at least 45 months post-transplant. In some embodiments, a method of treatment described herein results in a recipient having serum bilirubin values of 0.05-2 mg/dL, 0.1-1.5 mg/dL, 0.1-1.2 mg/dL, 0.5-1 mg/dL or 0.7-1 mg/dL for at least 50 months post-transplant. In some embodiments, a method of treatment described herein results in a recipient having serum bilirubin values of 0.05-2 mg/dL, 0.1-1.5 mg/dL, 0.1-1.2 mg/dL, 0.5-1 mg/dL or 0.7-1 mg/dL for at least 55 months post-transplant. In some embodiments, a method of treatment described herein results in a recipient having serum bilirubin values of 0.05-2 mg/dL, 0.1-1.5 mg/dL, 0.1-1.2 mg/dL, 0.5-1 mg/dL or 0.7-1 mg/dL for at least 60 months post-transplant.

In specific embodiments, a method of treatment described herein results in a recipient having a PT of 7-15 seconds, 8-14 seconds, 9-13 seconds, 9.4-12.5 seconds, or 10-12 seconds for at least 10 months, at least 15 months, at least 20 months, at least 25 months, at least 30 months, at least 35 months, at least 40 months, at least 45 months, at least 50 months, at least 55 months, or at least 60 months post-transplant. In some embodiments, a method of treatment described herein results in a recipient having a PT of 7-15 seconds, 8-14 seconds, 9-13 seconds, 9.4-12.5 seconds, or 10-12 seconds for at least 10 months post-transplant. In some embodiments, a method of treatment described herein results in a recipient having a PT of 7-15 seconds, 8-14 seconds, 9-13 seconds, 9.4-12.5 seconds, or 10-12 seconds for at least 15 months post-transplant. In some embodiments, a method of treatment described herein results in a recipient having a PT of 7-15 seconds, 8-14 seconds, 9-13 seconds, 9.4-12.5 seconds, or 10-12 seconds for at least 20 months post-transplant. In some embodiments, a method of treatment described herein results in a recipient having a PT of 7-15 seconds, 8-14 seconds, 9-13 seconds, 9.4-12.5 seconds, or 10-12 seconds for at least 25 months post-transplant. In some embodiments, a method of treatment described herein results in a recipient having a PT of 7-15 seconds, 8-14 seconds, 9-13 seconds, 9.4-12.5 seconds, or 10-12 seconds for at least 30 months post-transplant. In some embodiments, a method of treatment described herein results in a recipient having a PT of 7-15 seconds, 8-14 seconds, 9-13 seconds, 9.4-12.5 seconds, or 10-12 seconds for at least 35 months post-transplant. In some embodiments, a method of treatment described herein results in a recipient having a PT of 7-15 seconds, 8-14 seconds, 9-13 seconds, 9.4-12.5 seconds, or 10-12 seconds for at least 40 months post-transplant. In some embodiments, a method of treatment described herein results in a recipient having a PT of 7-15 seconds, 8-14 seconds, 9-13 seconds, 9.4-12.5 seconds, or 10-12 seconds for at least 45 months post-transplant. In some embodiments, a method of treatment described herein results in a recipient having a PT of 7-15 seconds, 8-14 seconds, 9-13 seconds, 9.4-12.5 seconds, or 10-12 seconds for at least 50 months post-transplant. In some embodiments, a method of treatment described herein results in a recipient having a PT of 7-15 seconds, 8-14 seconds, 9-13 seconds, 9.4-12.5 seconds, or 10-12 seconds for at least 55 months post-transplant. In some embodiments, a method of treatment described herein results in a recipient having a PT of 7-15 seconds, 8-14 seconds, 9-13 seconds, 9.4-12.5 seconds, or 10-12 seconds for at least 60 months post-transplant.

In specific embodiments, a method of treatment described herein results in a recipient having albumin values of 2-6 g/dL, 2.5-5.5 g/dL, 3-5 g/Dl, 3.5-5 g/dL, or 4-4.5 g/dL for at least 10 months, at least 15 months, at least 20 months, at least 25 months, at least 30 months, at least 35 months, at least 40 months, at least 45 months, at least 50 months, at least 55 months, or at least 60 months post-transplant. In some embodiments, a method of treatment described herein results in a recipient having albumin values of 2-6 g/dL, 2.5-5.5 g/dL, 3-5 g/Dl, 3.5-5 g/dL, or 4-4.5 g/dL for at least 10 months post-transplant. In some embodiments, a method of treatment described herein results in a recipient having albumin values of 2-6 g/dL, 2.5-5.5 g/dL, 3-5 g/Dl, 3.5-5 g/dL, or 4-4.5 g/dL for at least 15 months post-transplant. In some embodiments, a method of treatment described herein results in a recipient having albumin values of 2-6 g/dL, 2.5-5.5 g/dL, 3-5 g/Dl, 3.5-5 g/dL, or 4-4.5 g/dL for at least 20 months post-transplant. In some embodiments, a method of treatment described herein results in a recipient having albumin values of 2-6 g/dL, 2.5-5.5 g/dL, 3-5 g/Dl, 3.5-5 g/dL, or 4-4.5 g/dL for at least 25 months post-transplant. In some embodiments, a method of treatment described herein results in a recipient having albumin values of 2-6 g/dL, 2.5-5.5 g/dL, 3-5 g/Dl, 3.5-5 g/dL, or 4-4.5 g/dL for at least 30 months post-transplant. In some embodiments, a method of treatment described herein results in a recipient having albumin values of 2-6 g/dL, 2.5-5.5 g/dL, 3-5 g/Dl, 3.5-5 g/dL, or 4-4.5 g/dL for at least 35 months post-transplant. In some embodiments, a method of treatment described herein results in a recipient having albumin values of 2-6 g/dL, 2.5-5.5 g/dL, 3-5 g/Dl, 3.5-5 g/dL, or 4-4.5 g/dL for at least 40 months post-transplant. In some embodiments, a method of treatment described herein results in a recipient having albumin values of 2-6 g/dL, 2.5-5.5 g/dL, 3-5 g/Dl, 3.5-5 g/dL, or 4-4.5 g/dL for at least 45 months post-transplant. In some embodiments, a method of treatment described herein results in a recipient having albumin values of 2-6 g/dL, 2.5-5.5 g/dL, 3-5 g/Dl, 3.5-5 g/dL, or 4-4.5 g/dL for at least 50 months post-transplant. In some embodiments, a method of treatment described herein results in a recipient having albumin values of 2-6 g/dL, 2.5-5.5 g/dL, 3-5 g/Dl, 3.5-5 g/dL, or 4-4.5 g/dL for at least 55 months post-transplant. In some embodiments, a method of treatment described herein results in a recipient having albumin values of 2-6 g/dL, 2.5-5.5 g/dL, 3-5 g/Dl, 3.5-5 g/dL, or 4-4.5 g/dL for at least 60 months post-transplant.

In specific embodiments, a method of treatment described herein results in a recipient having an INR 1-3, 1.5-2.5, 1.5-2 or below 1.1 for at least 10 months, at least 15 months, at least 20 months, at least 25 months, at least 30 months, at least 35 months, at least 40 months, at least 45 months, at least 50 months, at least 55 months, or at least 60 months post-transplant. In some embodiments, a method of treatment described herein results in a recipient having an INR 1-3, 1.5-2.5, 1.5-2 or below 1.1 for at least 10 months post-transplant. In some embodiments, a method of treatment described herein results in a recipient having an INR 1-3, 1.5-2.5, 1.5-2 or below 1.1 for at least 15 months post-transplant. In some embodiments, a method of treatment described herein results in a recipient having an INR 1-3, 1.5-2.5, 1.5-2 or below 1.1 for at least 20 months post-transplant. In some embodiments, a method of treatment described herein results in a recipient having an INR 1-3, 1.5-2.5, 1.5-2 or below 1.1 for at least 25 months post-transplant. In some embodiments, a method of treatment described herein results in a recipient having an INR 1-3, 1.5-2.5, 1.5-2 or below 1.1 for at least 30 months post-transplant. In some embodiments, a method of treatment described herein results in a recipient having an INR 1-3, 1.5-2.5, 1.5-2 or below 1.1 for at least 35 months post-transplant. In some embodiments, a method of treatment described herein results in a recipient having an INR 1-3, 1.5-2.5, 1.5-2 or below 1.1 for at least 40 months post-transplant. In some embodiments, a method of treatment described herein results in a recipient having an INR 1-3, 1.5-2.5, 1.5-2 or below 1.1 for at least 45 months post-transplant. In some embodiments, a method of treatment described herein results in a recipient having an INR 1-3, 1.5-2.5, 1.5-2 or below 1.1 for at least 50 months post-transplant. In some embodiments, a method of treatment described herein results in a recipient having an INR 1-3, 1.5-2.5, 1.5-2 or below 1.1 for at least 55 months post-transplant. In some embodiments, a method of treatment described herein results in a recipient having an INR 1-3, 1.5-2.5, 1.5-2 or below 1.1 for at least 60 months post-transplant.

For example, the efficacy of a method of treatment described herein may be assessed by measuring renal function at 10 months, 15 months, 20 months, 25 months, 30 months, 35 months, 40 months, 45 months, 50 months, 55 months, or 60 months post-transplant. Renal function may be determined, for example, by measuring serum creatinine and/or glomerular filtration rate. In particular embodiments, a method described herein can result in a recipient having normal kidney function (as determined by serum creatinine and/or glomerular filtration rate (GFR)). In particular embodiments, a method described herein can result in a recipient having better kidney function (as determined by serum creatinine and/or glomerular filtration rate) as compared to a patient undergoing standard of care post-liver transplant therapy.

In specific embodiments, a method of treatment described herein results in a recipient having serum creatinine values of 0.5-1.5 mg/mL, 0.6-1.4 mg/mL, 0.7-1.3 mg/mL, 0.8-1.2 mg/mL, or 0.9-1.1 mg/mL for at least 10 months, at least 15 months, at least 20 months, at least 25 months, at least 30 months, at least 35 months, at least 40 months, at least 45 months, at least 50 months, at least 55 months, or at least 60 months post-transplant. In some embodiments, a method of treatment described herein results in a recipient having serum creatinine values of 0.5-1.5 mg/mL. In some embodiments, a method of treatment described herein results in a recipient having serum creatinine values of 0.6-1.4 mg/mL. In some embodiments, a method of treatment described herein results in a recipient having serum creatinine values of 0.7-1.3 mg/mL. In some embodiments, a method of treatment described herein results in a recipient having serum creatinine values of 0.8-1.2 mg/mL. In some embodiments, a method of treatment described herein results in a recipient having serum creatinine values of or 0.9-1.1 mg/mL for at least 10 months post-transplant. In some embodiments, a method of treatment described herein results in a recipient having serum creatinine values of at least 15 months post-transplant. In some embodiments, a method of treatment described herein results in a recipient having serum creatinine values of at least 20 months post-transplant. In some embodiments, a method of treatment described herein results in a recipient having serum creatinine values of at least 25 months post-transplant. In some embodiments, a method of treatment described herein results in a recipient having serum creatinine values of at least 30 months post-transplant. In some embodiments, a method of treatment described herein results in a recipient having serum creatinine values of at least 35 months post-transplant. In some embodiments, a method of treatment described herein results in a recipient having serum creatinine values of at least 40 months post-transplant. In some embodiments, a method of treatment described herein results in a recipient having serum creatinine values of at least 45 months post-transplant. In some embodiments, a method of treatment described herein results in a recipient having serum creatinine values of at least 50 months post-transplant. In some embodiments, a method of treatment described herein results in a recipient having serum creatinine values of at least 55 months post-transplant. In some embodiments, a method of treatment described herein results in a recipient having serum creatinine values of at least 60 months post-transplant.

In specific embodiments, a method of treatment described herein results in a recipient having a GFR of 40-90, 45-85, 50-80, 55-75, 60-70, or above 90 for at least 10 months, at least 15 months, at least 20 months, at least 25 months, at least 30 months, at least 35 months, at least 40 months, at least 45 months, at least 50 months, at least 55 months, or at least 60 months post-transplant. In some embodiments, a method of treatment described herein results in a recipient having a GFR of 40-90, 45-85, 50-80, 55-75, 60-70, or above 90 for at least 10 months post-transplant. In some embodiments, a method of treatment described herein results in a recipient having a GFR of 40-90, 45-85, 50-80, 55-75, 60-70, or above 90 for at least 15 months post-transplant. In some embodiments, a method of treatment described herein results in a recipient having a GFR of 40-90, 45-85, 50-80, 55-75, 60-70, or above 90 for at least 20 months post-transplant. In some embodiments, a method of treatment described herein results in a recipient having a GFR of 40-90, 45-85, 50-80, 55-75, 60-70, or above 90 for at least 25 months post-transplant. In some embodiments, a method of treatment described herein results in a recipient having a GFR of 40-90, 45-85, 50-80, 55-75, 60-70, or above 90 for at least 30 months post-transplant. In some embodiments, a method of treatment described herein results in a recipient having a GFR of 40-90, 45-85, 50-80, 55-75, 60-70, or above 90 for at least 35 months post-transplant. In some embodiments, a method of treatment described herein results in a recipient having a GFR of 40-90, 45-85, 50-80, 55-75, 60-70, or above 90 for at least 40 months post-transplant. In some embodiments, a method of treatment described herein results in a recipient having a GFR of 40-90, 45-85, 50-80, 55-75, 60-70, or above 90 for at least 45 months post-transplant. In some embodiments, a method of treatment described herein results in a recipient having a GFR of 40-90, 45-85, 50-80, 55-75, 60-70, or above 90 for at least 50 months post-transplant. In some embodiments, a method of treatment described herein results in a recipient having a GFR of 40-90, 45-85, 50-80, 55-75, 60-70, or above 90 for at least 55 months post-transplant. In some embodiments, a method of treatment described herein results in a recipient having a GFR of 40-90, 45-85, 50-80, 55-75, 60-70, or above 90 for at least 60 months post-transplant.

In certain embodiments, other assessments of the outcome of the transplant surgery can include the monitoring of the incidence of infection, the incidence of opportunistic infection, the onset of any treatment-related adverse events, and the patient's post-transplant quality of life. In certain embodiments, the efficacy of a method of treatment described herein may be determined by the recipient's requirement for immunosuppressive therapy. In particular embodiments, a method of treatment described herein results in no requirement for immunosuppressive therapy within 10 months, 15 months, 20 months, 25 months, 30 months, 35 months, 40 months, 45 months, 50 months, 55 months, or 60 months post-transplant. In some embodiments, a method of treatment described herein results in no requirement for immunosuppressive therapy within 10 months post-transplant. In some embodiments, a method of treatment described herein results in no requirement for immunosuppressive therapy within 15 months post-transplant. In some embodiments, a method of treatment described herein results in no requirement for immunosuppressive therapy within 20 months post-transplant. In some embodiments, a method of treatment described herein results in no requirement for immunosuppressive therapy within 25 months post-transplant. In some embodiments, a method of treatment described herein results in no requirement for immunosuppressive therapy within 30 months post-transplant. In some embodiments, a method of treatment described herein results in no requirement for immunosuppressive therapy within 35 months post-transplant. In some embodiments, a method of treatment described herein results in no requirement for immunosuppressive therapy within 40 months post-transplant. In some embodiments, a method of treatment described herein results in no requirement for immunosuppressive therapy within 45 months post-transplant. In some embodiments, a method of treatment described herein results in no requirement for immunosuppressive therapy within 50 months post-transplant. In some embodiments, a method of treatment described herein results in no requirement for immunosuppressive therapy within 55 months post-transplant. In some embodiments, a method of treatment described herein results in no requirement for immunosuppressive therapy within 60 months post-transplant.

In certain embodiments, functional assays can be used to detect biomarkers which are predictive of transplant rejection including the presence of circulating donor-specific antibodies (DSA) in the serum of the recipient which can be determined by ELISA. In certain embodiments, flow cytometric analyses on circulating lymphocytes can be performed to determine status of immune system reconstitution of the recipient. In certain embodiments, mixed lymphocyte reaction (MLR) of the recipient's peripheral blood mononuclear cells can be performed to assess the response of the recipient's cells to the donor cells and if the response changes after transplant surgery.

In certain embodiments, functional assays can be used to determine if induction of tolerance to the transplanted organ (e.g., liver) was achieved. These assays can include flow cytometric analysis to determine FoxP3+ T cells: CD4+ T cells ratio as an indicator for the presence of regulatory T-cells.

### 7.5 Equivalents and Incorporation by Reference

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

### 8. EXAMPLES

### 8.1 Example 1: A 60 Month, Single-Arm, Proof of Concept Study to Induce Immunological (e.g., Allogeneic) Tolerance in Deceased Donor Liver Transplant Recipients Using an Anti-CD2 Antibody, Siplizumab (TCD601), in Combination with Cyclophosphamide and Splenectomy

This example describes a clinical trial to evaluate certain embodiments of the methods of treatment described herein. Described herein is a 60 month, single-arm, proof of concept study to induce immunological tolerance in deceased donor liver transplant recipients using an anti-CD2 antibody, siplizumab (TCD601), in combination with cyclophosphamide and splenectomy.

### 8.1.1 Study Design

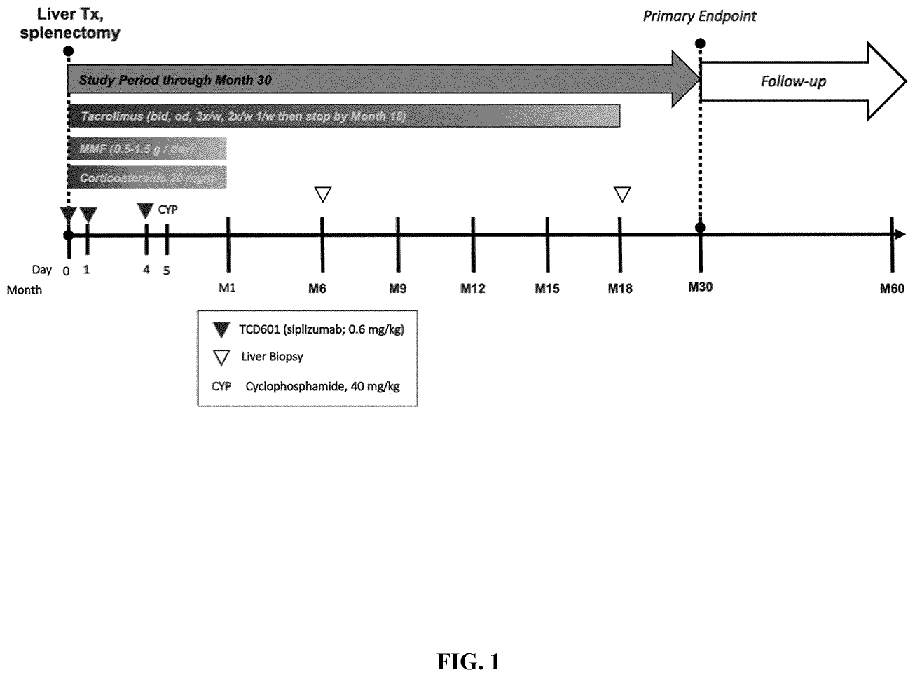

The study is a 60 month, single-arm, proof of concept study to induce allogeneic tolerance in deceased donor liver transplant recipients using an anti-CD2 antibody, siplizumab (TCD601), in combination with cyclophosphamide and splenectomy. The study is designed with the Primary Endpoint at 30 months and thereafter recipients will be followed with visits every 6 months until 5 years post-transplant. The study is planned to evaluate the ability of this siplizumab-based treatment regimen to induce tolerance (withdrawal of immunosuppression without allograft rejection and with preservation of function) in 12 adults who have received a deceased donor liver transplant. FIG. 1 shows an overview of the study.

During the pre-transplant screening period (Days −14 to −1), after informed consent has been signed, baseline subject information will be obtained, including date of birth (month and year), age, sex, race and ethnicity. In addition, relevant medical history (including cause of end-stage liver disease (ESLD)), current medical conditions at screening, a full physical examination, vital signs, laboratory assessments, and a pregnancy test (for females of child-bearing potential) will also be performed. Pre-transplant screening procedures (see inclusion and exclusion criteria in sections 8.1.3 and 8.1.4) will include ABO typing, anti-HLA antibodies and donor/recipient viral serology. Recipients who complete the screening period and meet all inclusion/exclusion criteria will enter the treatment period on their transplant date.

Patients will receive a liver transplant and splenectomy then be treated with standard of care immunosuppression of tacrolimus, mycophenolate and steroids, and mycophenolate and steroids will be stopped at Month 1. Siplizumab (0.6 mg/kg IV) will be given on Days 0, 1 and 4 and cyclophosphamide will be given on Day 5. Tacrolimus will be reduced gradually from Month 6 as indicated below if liver function is stable and the month 6 liver biopsy is rejection free. Tacrolimus will be completely stopped at Month 18, provided the Month 18 biopsy is free from rejection and the liver function remains stable. The primary endpoint will be evaluated at Month 30 and patients will remain in the study for follow-up until Month 60.

This study is based on the approach of using ex-vivo generated donor-specific regulatory T-cell therapy (Todo et al. (2016), Hepatology, 64:632-643) in living donor liver transplant recipients to induce immunological tolerance and allow withdrawal of immunosuppression. It is expected that blocking CD2 with this non-activating agent similarly will induce donor-specific regulatory T-cells, as well as depleting CD2-positive alloreactive cells, and induce tolerance to allow withdrawal of immunosuppression.

The purpose of this study is to investigate whether the encouraging results obtained with the ex vivo donor specific T regulatory cell approach in living donor liver transplantation (Todo et al. (2016), Hepatology, 64:632-643) can be reproduced in the deceased donor liver transplant population using siplizumab as the regulatory T-cell inducing agent. The overarching goal is freedom from immunosuppression with no liver allograft rejection and preservation of renal function.

The timing of siplizumab administration in this study is similar to that used in renal tolerance and the dose of 0.6 mg/kg is the same as in the living donor renal tolerance protocols (Kawai, et al. (2014), Am J Transplant 14:1599-1611). In the renal studies siplizumab was given on Days −2, −1, 0 and 1 at dose of 0.1, 0.6, 0.6 and 0.6 mg/kg. The timing of the doses in this liver transplant has been adapted to the deceased donor situation where pre-transplant interventions are necessarily limited. In addition, the liver transplant procedure, being more complex, may involve more fluid loss during surgery as well as potential reduction in the volume of distribution by ascites removal so the extension of the dosing period to Day 4 may mitigate this in terms of maintaining siplizumab exposure.

The duration of the study is 5 years, allowing the evaluation of the long-term benefits of withdrawal of immunosuppression to be evaluated. However, to evaluate the ability of the regimen to allow withdrawal from immunosuppression the primary endpoint will be assessed at Month 30. In addition, some post-transplant events such as chronic rejection present relatively late in the post-transplant course so these can be assessed during the extended follow-up.

### 8.1.2 Objectives and Endpoints

TABLE 2
Objectives and Related Endpoints

| Objective(s) | Endpoint(s) |
|---|---|
| Primary Objective(s) | Endpoints for primary objective |
| To evaluate the proportion of patients | Immunosuppression dosing |
| who are free from immunosuppression, | Incidence of tBPAR (Liver |
| without treated biopsy proven acute rejection | biopsy) |
| (tBPAR), at Month 30 post-transplant | |
| | Endpoints for key secondary |
| Key Secondary Objective | objective |
| Composite efficacy failure rate of | Incidence of tBPAR (Liver |
| treated biopsy-proven acute rejection | biopsy) |
| (tBPAR), graft loss or death at | Patient/graft survival and |
| Month 30 | disposition |
| Secondary Objectives | Endpoints for secondary objectives |
| Glomerular Filtration Rate (GFR) and rate of | Serum creatinine, demographics |
| change in renal function at Month 30 | Liver biopsy |
| Incidence, severity and treatment of acute | Patient/graft survival |
| rejection | AE/SAE infections |
| Death | Laboratory data |
| Graft Loss | |
| Adverse events (AE), Serious adverse events | |
| (SAEs), including infections | |
| Laboratory values (liver function tests (LFTs), | |
| International Normalized Ratio (INR)) | |
| | Endpoints for exploratory |
| Exploratory Objectives | objectives |
| Pharmacodynamics (PD) of siplizumab | White blood cell count, flow |
| post-transplant | cytometry (lymphocyte |
| | phenotyping) |

### 8.1.3 Inclusion Criteria

Subjects eligible for inclusion in this study have to fulfill all of the following criteria:
1. Adult aged 18-70 receiving an ABO compatible deceased donor liver transplant.
2. MELD score <30 on recent evaluation with 60 days of screening
3. Stable cardio-pulmonary status per the judgement of the investigator and eligible for transplantation per local regulations.
4. EBV seropositive.
5. Covid-19 PCR negative.
6. Male study subjects willing to maintain barrier contraception (condom) and agree not to father a child until 12 weeks after the last dose of MMF.
7.

### 8.1.4 Exclusion Criteria

Subjects meeting any of the following criteria are not eligible for inclusion in this study.
1. Use of other investigational drugs (or enrollment in another investigational drug study) within 30 days of screening or 5 half-lives of the medication, whichever is longer
2. History of hypersensitivity to any of the study treatments or their excipients or to drugs of similar chemical classes (e.g., siplizumab, tacrolimus, cyclophosphamide or mycophenolate)
3. End stage liver disease of autoimmune origin, including autoimmune hepatitis, primary biliary cholangitis or primary sclerosing cholangitis.
4. Subjects with leukopenia (WBC less than 2,000/mm³) or thrombocytopenia (platelet count <70,000/mm³) at baseline
5. Sero-positive for HIV-1 or HBsAg. Subjects who are sero-positive for Hepatitis C virus are excluded without proof of sustained viral response (SVR) after anti-HCV treatment
6. Subjects with a history of TB or latent TB infection as detected by Quantiferon Gold Plus IGRA (or current standard interferon gamma release assay for TB)
7. Subjects with extrahepatic malignancy or history of same, other than basal cell carcinoma of the skin or carcinoma in situ of the cervix.
8. Cardiac ejection fraction ≤40% within 6 months or clinical evidence of cardiac insufficiency
9. Subjects who, in the opinion of the investigator, are not capable of giving informed consent for the study or who are unable or unwilling to adhere to the study requirement outlined in the protocol.
10. Subjects with any other clinically significant medical condition or laboratory abnormality that would, in the judgment of the investigator interfere with the subject's ability to participate in the study.
11. Subjects who have received any live-attenuated vaccine within 2 months of planned transplant.
12. Pregnant or nursing (lactating) women, where pregnancy is defined as the state of a female after conception and until the termination of gestation, confirmed by a positive human chorionic gonadotropin (hCG) laboratory test.
13. Women of child-bearing potential, defined as all women physiologically capable of becoming pregnant, unless they are using highly effective methods of contraception during dosing and for 24 weeks after the study medications have been stopped. Highly effective contraception methods include:
   Total abstinence (when this is in line with the preferred and usual lifestyle of the subject. Periodic abstinence (e.g., calendar, ovulation, symptothermal, post-ovulation methods) and withdrawal are not acceptable methods of contraception.
   Female sterilization (have had surgical bilateral oophorectomy with or without hysterectomy) or tubal ligation at least six weeks before taking study treatment. In case of oophorectomy alone, only when the reproductive status of the woman has been confirmed by follow up hormone level assessment.
   Male sterilization (at least 6 months prior to screening). For female subjects on the study, the vasectomized male partner should be the sole partner for that subject.
   Any of the following:
      a. Use of oral, injected or implanted hormonal methods of contraception or other forms of hormonal contraception that have comparable efficacy (failure rate <1%), for example, hormone vaginal ring or transdermal hormone contraception.
      b. Placement of long-acting reversible contraceptives an intrauterine device or intrauterine system.

In case of use of oral contraception women should have been stable on the same brand (or generic equivalent) for a minimum of 3 months before taking study treatment.

Women are considered post-menopausal and not of childbearing potential if they have had 12 months of natural (spontaneous) amenorrhea with an appropriate clinical profile (e.g., age appropriate, history of vasomotor symptoms) or have had surgical bilateral oophorectomy (with or without hysterectomy) or tubal ligation at least six weeks prior. In the case of oophorectomy alone, only when the reproductive status of the woman has been confirmed by follow up hormone level assessment is she considered not of childbearing potential.

### 8.1.5 Treatments

(a) TCD601 (siplizumab)

TCD601 (siplizumab) is a humanized IgG1/x class monoclonal antibody, supplied as a clear to slightly opalescent, colorless to slightly brownish, preservative-free solution for infusion.

The dose of TCD601 will be 0.6 mg/kg at each administration.

The planned duration of treatment with TCD601 is three days. Subjects will receive a total of three doses of TCD601 peri-operatively on Days 0, 1 and 4. Subjects may be discontinued from treatment earlier at the discretion of the investigator or upon request of the subject. TCD601 dose adjustments and/or interruptions for a given subject are not permitted. The TCD601 infusion rate may be changed in the event of an infusion reaction.

The dose will be calculated by the following formula: Dose (mg)=[patient weight (kg)×dose level (mg/kg)]

Upon confirmation of subject dose, the product will be prepared from vial(s), using aseptic technique and administration to the subject via IV infusion using a syringe or infusion pump.

(i) Premedication for Siplizumab

At the first dose of siplizumab on Day 0 a steroid bolus must have been given prior to starting the infusion, this should be done as part of pre-operative steroids management see below. Prior to every infusion of TCD601, subjects should receive premedication with 650-1000 mg acetaminophen (or paracetamol) and an H₁-antagonist (antihistamine; e.g. 25 mg diphenhydramine) to minimize signs and symptoms of an infusion reaction. Administration of required premedications should occur at least 30 min-1 hour and no more than 5 hours prior to the start of the infusion.

(ii) Siplizumab Administration (Days 0, 1 and 4)

The first dose of siplizumab (0.6 mg/kg) will be administered on Day 0 pre- or intra-operatively and timed so that the completion of the infusion occurs no earlier than 4 hours prior to the planned revascularization and perfusion of the allograft. Pre-medication should be coordinated with any pre-operative medications to avoid therapeutic duplication in consultation with the anesthesiologist. When administered intra-operatively, the infusion must be completed prior to revascularization. This ensures both recipient and donor T-lymphocytes are suppressed, and depletion is initiated immediately post-transplant. The second siplizumab dose on Day 1 should be administered approximately 20-24 hours after the Day 0 dose. The third and final dose should occur at approximately the same time of day on Day 4 as was the Day 1 dose. Subjects should be carefully monitored (a minimum of 2-hours) after each dose for infusion reactions.

For all doses, the diluted siplizumab solution is infused intravenously using a syringe pump over a period of around 1 hour. Pre-medication (see above) must precede administration of siplizumab. This procedure should be followed for each of the 2 subsequent siplizumab infusions on Days 1 and 4. The infusions may be given directly into a peripheral vein or a separate lumen in an indwelling, multi-lumen, central catheter and not administered concurrently with other medications. No dose adjustments beyond changes based on the subject's actual weight are permitted to the TCD601 dose during the study.

(iii) Management of First Dose (Infusion) Reactions

“First dose reactions” may occur upon the initial administration of siplizumab. These have the potential to be severe.

Although premedications are given prophylactically prior to administration of siplizumab, further doses of acetaminophen and antihistamine may be administered if fever or chills occur post-study drug administration. Corticosteroids (250 mg IV of methylprednisolone) may be administered if a severe reaction occurs.
(b) Cyclophosphamide (Day 5)

Prior to administration of cyclophosphamide, the recipient should be sedated and pre-treated with for example dexamethasone, an antihistamine (e.g. diphenhydramine), benzodiazepine per local practice (e.g., lorazepam), and granisetron or alternate anti-emetic per local institutional standards to prevent nausea.

Participants will receive cyclophosphamide, 40 mg/kg/day (based on lesser of ideal or actual body weight) on Day 4 or Day 5; cyclophosphamide is dissolved in sterile water or 0.9% sodium chloride for injection and diluted in e.g. 250 cc dextrose and infused over 1 hour. Cyclophosphamide is administered via intravenous infusion.

Prophylaxis against hemorrhagic cystitis using MESNA, 15 mg/kg IV bolus will be administered 15 minutes before and 3, 6 and 9 hours after cyclophosphamide plus IV hydration (e.g. with 5% dextrose, 1500 mL/M²/24 hrs as tolerated) beginning 4 hours prior to cyclophosphamide.

(c) Splenectomy (Day 0)

At the end of transplant surgery, before abdominal wall closure, total splenectomy will be performed by ligation and division of splenic vasculature close to the splenic parenchyma.

(d) Concomitant Immunosuppression

The use of immediate release brand (Prograf) or generic TAC is allowed in this study. Subjects should remain on the same concomitant immunosuppression medication throughout the duration of the study (12 months), i.e. if a subject starts with a branded drug they should remain on brand and not switch to a generic at any point throughout the study; if a subject starts with generic, the subject should remain on the same generic throughout the study.

Special attention must be taken to avoid concomitant administration of drugs, food, or beverages which are known to be a strong inducer or inhibitor of CYP3A4.

In general, symptomatic treatment should be considered first to treat subjects who have difficulties tolerating their immunosuppressive regimen. However, for subjects who are still unable to tolerate the protocol-specified study treatment, dose adjustments, route adjustments, and interruptions of study drugs may be permitted in order to keep the subject on study treatment.

Concomitant medications will be supplied locally and used according to the local label and will include:
Immediate release TAC as 0.5 mg, 1.0 mg, or 5.0 mg capsules,
Mycophenolate as 500 or 250 mg capsules
CS for oral and IV administration

Pre-transplant immunosuppression may be administered according to center practice, but such practice must be applied consistently to all subjects at a given center.

(i) TAC Administration

TAC will be administered as capsules or tablets orally (P.O.) twice a day (BID) and adjusted to maintain serum trough (Co) concentrations within the target range of 4-11 ng/mL. If oral administration is not feasible or practical IV TAC containing the equivalent of 5 mg/mL tacrolimus administration by continuous intravenous infusion can be substituted per label. Once a day TAC is not permitted.

TAC should be initiated as soon as possible in the peri-transplant period and may follow local practice but must be initiated no later than 24 hours after reperfusion of the allograft. The lowest permitted dosing of TAC in this study is 0.5 mg BID or IV equivalent. In case of TAC intolerance, where the study regimen cannot be maintained, the subject may be switched to cyclosporine or mTor inhibitors and remain in the trial. If the conditions for withdrawal of immunosuppression continue to be met cyclosporine or mTor inhibitor may be withdrawn following the schedule below.

In the event of TAC intolerance (e.g., nephrotoxicity, neurotoxicity) dose reduction of TAC may be necessary. TAC is a substrate for CYP3A metabolism and therefore susceptible to drug-drug interactions that can raise or lower systemic concentrations, leading to toxicity or therapeutic failure. The co-administration of drugs known to interfere with TAC metabolism should be avoided if possible.

(ii) Tacrolimus Weaning Protocol
1. When the patient has a clean Month 6 biopsy and has maintained stable graft function during 3 preceding months, weaning from IS therapy will be initiated at 6 months post-liver transplantation (LT).
2. First the twice-daily Tac dose (or corresponding CyA or mTOR) will be reduced to once a day at three quarters (75%) of the daily dose over three months to Month 9.
3. From Month 9-12 a subsequent reduction at same dose 3 times per week
4. From Month 12-15 a subsequent reduction at same dose 2 times/week,
5. From Month 15-18 a subsequent reduction to 1 time/week.
6. Provided the Month 18 biopsy is clean the calcineurin inhibitor (CNI) or mTOR based immunosuppression will be completely stopped at 18 months.

During weaning and immediately thereafter to Month 24, additional liver labs (ALT, GGT and bilirubin) may be taken at the discretion of the investigator e.g. every 2 weeks for safety monitoring.

(e) Mycophenolate Administration (Day 0 to Month 1 Only)

Mycophenolate will be administered orally twice per day (BID) for a total daily dose of 500-1500 mg/day (from 250 mg BID to 750 mg BID). Where oral administration is not feasible, as 1000 mg/day via IV administration (2-500 mg vial for IV administration). For subjects who remain intubated >24 hours post-transplant and/or whom are otherwise unable to swallow oral medication, IV mycophenolate may be substituted 1:1 until oral conversion is possible. The first dose of mycophenolate will be administered no later than 24 hours after graft reperfusion of the liver, mycophenolate should be stopped at Month 1 post-transplant.

(f) Corticosteroid Administration (Day 0 to Month 1 Only)

Corticosteroids (CS) are given at several times in the study for different purposes and the prespecified dosing guidance is as follows below.
Peri-operative immunosuppression-Solumendrol—Day 0: 500 mg IV before transplant and 500 mg IV at revascularization.
Solumendrol IV taper—e.g. Day 1: 160 mg; Day 2: 125 mg; Day 3: 100 mg; Day 4: 80 mg; and Day 5: 40 mg.
Transition to oral treatment: Day 6: 20 mg prednisolone daily divided into two 10 mg doses until Month 1 when it is stopped.

If rejection is observed steroids should be used as first line treatment.

(g) Infectious Prophylaxis

(i) Antimicrobial Prophylaxis for Surgery

Prophylactic antimicrobial therapy will be administered prior to surgery according to local practice.

(ii) CMV Prophylaxis and Treatment

Cytomegalovirus prophylaxis is central to the successful outcome of organ transplantation. Given that CMV infection may occur in any combination (including D+/R+ and D−/R− with transfusion) a uniform approach is most reasonable in terms of preventing CMV infection. All recipients will have their pre-transplant CMV serologic status obtained for donor and recipient.

All recipients will receive daily ganciclovir, valganciclovir or the locally approved CMV prophylaxis regimen, starting 10 days post-transplant at the latest and continuing until 3 months post-transplant. Dose-adjustments will be performed according per label as needed. Note that during periods of neutropenia, valganciclovir/ganciclovir should not be used and should be switched to letermovir or equivalent as available.

(iii) Treatment of Symptomatic CMV Disease

In case of symptomatic CMV disease a minimum treatment of 2-3 weeks of intravenous ganciclovir at a dose of e.g. 5 mg/kg twice daily or oral valganciclovir, or other locally approved antiviral medication should be given. CMV IgG may be also be added for seronegative subjects with viral activation. The end point of intravenous therapy is the documented clearance of virus from the blood as demonstrated by CMV antigenemia assay (or quantitative PCR assay).

(iv) Pneumocystis jirovecii (Pneumocystis carinii) Pneumonia (PCP)

Sulfamethoxazole and Trimethoprim, (SMX-TMP), 1 single strength tablet daily starting immediately post-transplant, when kidney function has stabilized, and continuing for the first 6 months post-transplant will be administered to all participants. Dosing level adjusted for kidney function as per label. In the event of allergy or intolerance to the components of TMP/SMX, Pentacarinat (pentamidine) inhalations, 300 mg every four weeks, will be used in its place for prophylaxis of Pneumocystis carinii. Because of the potential for bone marrow suppression with TMP/SMX, subjects must be monitored carefully during the first month post-transplant, and the alternative regimen used in the case of possible TMP/SMX-related bone marrow suppression. Consideration will be given to extending the period of prophylaxis for an additional 6 months if it is felt to be clinically indicated, since TMP/SMX provides prophylaxis against the majority of isolates of pneumococcus and H. influenzae.

(v) Hepatitis B Virus (HBV)

Prophylaxis for HBV reactivation during the course of this study is allowed and will be administered at the discretion of the Investigator.

(vi) Fungal/Yeast Infection Prophylaxis

Fluconazole will be started on transplant Day 1 and continued at least until resolution of neutropenia.

(vii) Oral Candida

For oral thrush (Candida), Nystatin may be used in a swish and swallow regimen; alternatively, clotrimazole (Mycelex®) lozenges/troches may be used. Routine use of systemic agents, e.g., itraconazole, voriconazole and fluconazole, will not be allowed except as above in the immediate post-transplant period or if patients are systemically infected. Attention should be paid to the interaction of azole antifungal agents which may increase blood concentrations of TAC.

Systemic therapy will be based on center practice and at the Investigator's discretion.

(h) Treatment of Acute Rejection Episodes

In all suspected acute rejection episodes, regardless of initiation of anti-rejection treatment, a liver biopsy should be performed within 48 hours.

Acute rejections should be treated with bolus methylprednisolone (other CS are acceptable at an equivalent dose) according to local practice. Recommended treatment is at least 3 boluses of IV methylprednisolone with a minimal dose of 250 mg/bolus or at least 2 boluses of IV methylprednisolone with a minimal total dose of 750 mg.

Subjects who experience steroid-resistant rejection, vascular rejection, antibody mediated rejection or rejection with a Banff RAI≥7 (Demetris 2016) may be treated with other anti-rejection therapies (i.e., antibody therapy).

It is important to note that the combination of TCD601 and other T-cell depleting antibodies, e.g., ATG, alemtuzumab, mTor inhibitors (sirolimus or everolimus), IVIG or costimulatory blockade (belatacept) have not been investigated and may result in overlapping pharmacology. In the setting of an acute rejection episode, the selection and use of one or more of these agents in combination with TCD601 is at the investigator's discretion based on their experience.

(i) Immunization

Immunization of transplant candidates for vaccine preventable diseases is recommended more than 2 weeks prior to transplantation or starting at 1-6 months after transplantation. If given prior to transplantation, the full immunization series should be completed before the transplant procedure. In certain situations, it may be appropriate to wait until 3 or more months after transplantation to vaccinate, such as following T- or B-cell depletion therapy. Waning vaccine titers to other routine immunizations have been well documented after transplantation. Lower seroconversion rates to influenza vaccination are well documented in the setting of mycophenolate mofetil and TAC use (Danziger-Isakov and Kumar 2019).

Vaccination during treatment with siplizumab and prior to clearance of the antibody and pharmacodynamic effects may result in therapeutic failure (i.e., non-protective antibody titers). Administration of live attenuated agents should be avoided while receiving siplizumab treatment and for up to 6 months thereafter, depending on the dose and time for reconstitution of immune function.

(j) EBV Post-Transplant Lymphoproliferative Disease (PTLD)

EBV-PTLD is a serious disease. Transplant clinicians managing suspected and confirmed PTLD should follow local institutional guidance on the diagnosis and management of EBV-PTLD, including consulting with transplant infectious disease.

In patients diagnosed with EBV-PTLD, therapy must be individualized. doses planned for this trial are expected to result in T- and NK-cell depletion and minimal to no modulation of the B-cell compartment, respectively. Clinicians should refer to the siplizumab IB when considering therapeutic options for the treatment of EBV-PTLD.

Consensus guidelines on the diagnosis and management of EBV-LPD and PTLD in the setting of solid organ transplantation have been published and may be referenced (Allen et al. (2013), Am J Transplant; 13 Suppl 4:107-120. doi:10.1111/ajt.12104; Parker et al. (2010), Br J Haematol; 149(5):675-692. doi:10.1111/j.1365-2141.2010.08161.x).

(k) Prohibited Treatment

Immunosuppressants and induction treatment other than those specified in the protocol are NOT allowed after informed consent up to the end of study. If the use of any of these medications or other non-protocol immunosuppressants is discovered prior to randomization/enrollment, the subject must not be randomized and will be recorded as a screen failure. If discovered after randomization/enrollment, no further doses are to be given, and the subject should continue on the randomized/assigned treatment regimen, noting the protocol deviation.

The exception is for the treatment of acute rejection not responding to corticosteroids.

The use of concomitant medications that may influence tacrolimus levels should be avoided.

### 8.2 Efficacy Assessments

Timing of efficacy assessments are shown in Table 3.

TABLE 3
Schedule of Assessments

| | STUDY PERIOD | | | | | | | | | | | | | | | | |
|---|---|---|---|---|---|---|---|---|---|---|---|---|---|---|---|---|---|
| | | TREATMENT | | | | | | | | | | | | | | | |
| | SCREENING | Immunosuppression | | | | | | | | | | | | Weaning^{m} | | | |
| | Visit Name (Day/Month) | | | | | | | | | | | | | | | | |
| | Day −14 | D 0 | D | D | D | D | D | D | M | M | M | M | M | M | M | M | M |
| | to 0^{a} | LTX | 1 | 4 | 5 | 7 | 14 | 21 | 1 | 2 | 3 | 4 | 5 | 6 | 7.5 | 9 | 10.5 |
| General Assessments | | | | | | | | | | | | | | | | | |
| Informed Consent | X | | | | | | | | | | | | | | | | |
| Inclusion/Exclusion | X | X | | | | | | | | | | | | | | | |
| Demographics | X | | | | | | | | | | | | | | | | |
| Medical History | X | | | | | | | | | | | | | | | | |
| Physical | X | X | | | | | | | X | | | | | X | | | |
| Examination | | | | | | | | | | | | | | | | | |
| Vital Signs^{b} | X | X | X | X | X | X | X | X | X | X | X | X | X | X | X | X | X |
| Prior & | As | | | | | | | | | | | | | | | | |
| Concomitant | needed | | | | | | | | | | | | | | | | |
| Medications | | | | | | | | | | | | | | | | | |
| Adverse Events | | As needed | | | | | | | | | | | | | | | |
| Histocompatibility | | | | | | | | | | | | | | | | | |
| and Infectious | | | | | | | | | | | | | | | | | |
| Disease Testing | | | | | | | | | | | | | | | | | |
| (Donor + Recipient) | | | | | | | | | | | | | | | | | |
| ABO typing | X | | | | | | | | | | | | | | | | |
| CMV, EBV, HBV, | X | | | | | | | | | | | | | | | | |
| HCV, HIV, | | | | | | | | | | | | | | | | | |
| COVID19 | | | | | | | | | | | | | | | | | |
| COVID 19 PCR | X | | | | | | | | | | | | | | | | |
| recipient | | | | | | | | | | | | | | | | | |
| Laboratory | | | | | | | | | | | | | | | | | |
| Assessments | | | | | | | | | | | | | | | | | |
| Hematology^{c} | X | X | X | X | X | X | X | X | X | X | X | X | X | X | | X | |
| Chemistry^{d, o} | X | X | X | X | X | X | X | X | X | X | X | X | X | X | X | X | X |
| Coagulation^{e} | X | X | X | X | X | X | X | X | X | | | | | X | | | |
| Lipids and HbA1c | X | | | | | | | | | | | | | X | | | |
| Urine Pregnancy | | X | X | | | | | | | | | | | X | | | |
| testing | | | | | | | | | | | | | | | | | |
| Serum Pregnancy | X | | | | | | | | | | | | | | | | |
| testing | | | | | | | | | | | | | | | | | |
| Prophylaxis | | | | | | | | | | | | | | | | | |
| Hemorrhagic | | | | | X | | | | | | | | | | | | |
| cystitis^{f} | | | | | | | | | | | | | | | | | |
| CMV prophylaxis^{g} | | X^{g} | | | | | | | | | | | | | | | |
| Pneumocystis | | X | | | | | | | | | | | | | | | |
| jirovecii pneumonia | | | | | | | | | | | | | | | | | |
| Fungal/Yeast | | X | | | | | | | | | | | | | | | |
| prophylaxis | | | | | | | | | | | | | | | | | |
| Oral candida | | As needed | | | | | | | | | | | | | | | |
| prophylaxis | | | | | | | | | | | | | | | | | |
| Pre-operative | | X | | | | | | | | | | | | | | | |
| infectious | | | | | | | | | | | | | | | | | |
| prophylaxis | | | | | | | | | | | | | | | | | |
| Study Treatment | | | | | | | | | | | | | | | | | |
| Regimen | | | | | | | | | | | | | | | | | |
| TCD601 | | X | X | X | | | | | | | | | | | | | |
| (siplizumab)^{h} | | | | | | | | | | | | | | | | | |
| Tacrolimusⁱ | | X | X | X | X | X | X | X | X | X | X | X | X | X | X | X | X |
| Tacrolimus | | | X | X | X | X | X | X | X | X | X | X | X | X | X | X | X |
| concentrationsⁱ | | | | | | | | | | | | | | | | | |
| MMF^{j} | | X | X | X | X | X | X | X | | | | | | | | | |
| Cyclophosphamide | | | | | X | | | | | | | | | | | | |
| Corticosteroids^{k} | | X | X | X | X | X | X | X | | | | | | | | | |
| Disease Monitoring | | | | | | | | | | | | | | | | | |
| Liver Biopsy^{l} | | X | | | | | | | | | | | | X | | | |
| Rejection | | As needed | | | | | | | | | | | | | | | |
| Graft Loss | | As needed | | | | | | | | | | | | | | | |
| Mechanistic Studies | | | | | | | | | | | | | | | | | |
| Lymphocyte | | X | | | | x | | | X | | | | | X | | | |
| phenotyping | | | | | | | | | | | | | | | | | |

| | STUDY PERIOD | | | | | | | | | | | | | | | |
|---|---|---|---|---|---|---|---|---|---|---|---|---|---|---|---|---|
| | TREATMENT | | | | | | | | | | | FOLLOW-UP | | | | |
| | Weaning^{m} | | | | Immunosuppression free^{m} | | | | | | | | | | | |
| | Visit Name (Day/Month) | | | | | | | | | | | | | | | |
| | M | M | M | M | M | M | M | M | M | M | M | M | M | M | M | M 60/ |
| | 12 | 13.5 | 15 | 16.5 | 18 | 19.5 | 21 | 22.5 | 24 | 27 | 30 | 36 | 42 | 48 | 54 | EOS/ET |
| General Assessments | | | | | | | | | | | | | | | | |
| Informed Consent | | | | | | | | | | | | | | | | |
| Inclusion/Exclusion | | | | | | | | | | | | | | | | |
| Demographics | | | | | | | | | | | | | | | | |
| Medical History | | | | | | | | | | | | | | | | |
| Physical | X | | | | | | X | | | | X | | | | | X |
| Examination | | | | | | | | | | | | | | | | |
| Vital Signs^{b} | X | X | X | X | X | X | X | X | X | X | X | X | X | X | X | X |
| Prior & | | | | | | | | | | | | | | | | |
| Concomitant | | | | | | | | | | | | | | | | |
| Medications | | | | | | | | | | | | | | | | |
| Adverse Events | As needed | | | | | | | | | | | | | | | |
| Histocompatibility | | | | | | | | | | | | | | | | |
| and Infectious | | | | | | | | | | | | | | | | |
| Disease Testing | | | | | | | | | | | | | | | | |
| (Donor + Recipient) | | | | | | | | | | | | | | | | |
| ABO typing | | | | | | | | | | | | | | | | |
| CMV, EBV, HBV, | | | | | | | | | | | | | | | | |
| HCV, HIV, | | | | | | | | | | | | | | | | |
| COVID19 | | | | | | | | | | | | | | | | |
| COVID 19 PCR | | | | | | | | | | | | | | | | |
| recipient | | | | | | | | | | | | | | | | |
| Laboratory | | | | | | | | | | | | | | | | |
| Assessments | | | | | | | | | | | | | | | | |
| Hematology^{c} | X | | X | | X | | X | | X | X | X | X | X | X | X | X |
| Chemistry^{d, o} | X | X | X | X | X | X | X | X | X | X | X | X | X | X | X | X |
| Coagulation^{e} | X | | | | | | | | X | X | X | | | X | | X |
| Lipids and HbA1c | X | | | | | | | | X | | X | | | | | X |
| Urine Pregnancy | X | | | | X | | | | X | | | | | | | |
| testing | | | | | | | | | | | | | | | | |
| Serum Pregnancy | | | | | | | | | | | X | | | | | X |
| testing | | | | | | | | | | | | | | | | |
| Prophylaxis | | | | | | | | | | | | | | | | |
| Hemorrhagic | | | | | | | | | | | | | | | | |
| cystitis^{f} | | | | | | | | | | | | | | | | |
| CMV prophylaxis^{g} | | | | | | | | | | | | | | | | |
| Pneumocystis | | | | | | | | | | | | | | | | |
| jirovecii pneumonia | | | | | | | | | | | | | | | | |
| Fungal/Yeast | | | | | | | | | | | | | | | | |
| prophylaxis | | | | | | | | | | | | | | | | |
| Oral candida | As needed | | | | | | | | | | | | | | | |
| prophylaxis | | | | | | | | | | | | | | | | |
| Pre-operative | | | | | | | | | | | | | | | | |
| infectious | | | | | | | | | | | | | | | | |
| prophylaxis | | | | | | | | | | | | | | | | |
| Study Treatment | | | | | | | | | | | | | | | | |
| Regimen | | | | | | | | | | | | | | | | |
| TCD601 | | | | | | | | | | | | | | | | |
| (siplizumab)^{h} | | | | | | | | | | | | | | | | |
| Tacrolimusⁱ | X | X | X | X | X | | | | | | | | | | | |
| Tacrolimus | X | X | X | X | X | | | | | | | | | | | |
| concentrationsⁱ | | | | | | | | | | | | | | | | |
| MMF^{j} | | | | | | | | | | | | | | | | |
| Cyclophosphamide | | | | | | | | | | | | | | | | |
| Corticosteroids^{k} | | | | | | | | | | | | | | | | |
| Disease Monitoring | | | | | | | | | | | | | | | | |
| Liver Biopsy^{l} | | | | | X | | | | | | X | | | | | |
| Rejection | As needed | | | | | | | | | | | | | | | |
| Graft Loss | As needed | | | | | | | | | | | | | | | |
| Mechanistic Studies | | | | | | | | | | | | | | | | |
| Lymphocyte | X | | | | X | | | | X | | | | | | | |
| phenotyping | | | | | | | | | | | | | | | | |

Footnotes
SD = noted in source document,
LTX = Liver transplant,
EOS/ET = end of study or early termination
^{a}screening may occur on the same day as liver transplant (Day 0)
^{b}Vital signs include Blood pressure, pulse, temperature and weight, height will be collected at the first visit only.
^{c}Hemoglobin, hematocrit, red blood cell (RBC), MCV, MCH, Platelet Count, white blood cell (WBC) and differential count (% and abs).
^{d}Albumin, total bilirubin, calcium, creatinine, glucose, phosphorus, magnesium, potassium, ALT, AST, ALP, GGT, amylase, sodium, bicarbonate, urea/BUN.
^{e}PT/INR, PTT
^{f}MESNA for prophylaxis in conjunction with cyclophosphamide dosing.
^{g}CMV prophylaxis starting 10 days post-transplant at the latest and continuing until 3 months post-transplant.
^{h}on Day 0 a steroid bolus must have been given prior to the first infusion of siplizumab. Prior to EVERY infusion of siplizumab, subjects should receive premedication with 650-1000 mg acetaminophen (or paracetamol) and an H1-antagonist (antihistamine).
ⁱTAC should be initiated as soon as possible in the peri-transplant period and adjusted to target of 4-11 ng/mL using local laboratory trough (C0) levels. Weaning of TAC will take place between Months 6 and 18, provided the patient meets the criteria.
^{j}MMF will be started no later than 24 hours after graft reperfusion of the liver and stopped at Month 1 post-transplant.
^{k}Peri-Transplant Solumedrol −Day 0: 500 mg IV before Transplant and at 500 mg IV at reperfusion. Solumedrol IV taper- e.g. Day 1, 160 mg, D 2 125 mg, D 3 100 mg, D 4 80 mg, D 5 40 mg. Day 6 20 mg prednisolone daily until Month 1 when it is stopped
^{l}Liver biopsies per protocol at Baseline, Months 6 and 18 to allow weaning and Month 30 for primary analysis. For cause biopsy to be performed for all suspected rejections.
^{m}During weaning and immediately thereafter to Month 24, additional labs should be taken every 2 weeks for ALT, alkaline phosphatase and bilirubin.

### 8.2.1 Treated Biopsy Proven Acute Rejection

Treated biopsy proven acute rejection (tBPAR) is any condition in which the subject receives anti-rejection treatment and is histologically diagnosed as acute rejection according to the 2016 Banff criteria, including borderline rejections, as described in Demetris et al., Am J Transplant. 2016 October; 16(10):2816-2835.

### 8.2.2 Graft Loss

The allograft will be presumed to be lost on the day the subject has irreversible acute liver failure or dies due to liver failure. If the subject undergoes retransplantation then the retransplant date will be recorded as the day of graft loss. A patient who dies with their allograft function intact will not be considered to have suffered graft loss.

### 8.3 Safety Assessments

All blood samples will be taken by either direct venipuncture or an indwelling cannula.

### 8.3.1 Physical Examination

A complete physical examination (as per visit schedule) will include a comprehensive assessment of the subject's general appearance, skin, neck (including thyroid), eyes, ears, nose, throat, lungs, heart, abdomen (including spleen and liver), back, lymph nodes, extremities and vasculature. The examination will also include a neurological evaluation. If indicated based on medical history and/or symptoms, rectal, external genitalia, breast, and pelvic exams will be performed.

### 8.3.2 Vital Signs

Vital signs (radial pulse rate, blood pressure and body temperature) will be recorded as indicated in the Schedule of Assessments (see Table 3). Attempts should be made to assess the blood pressure and pulse on the same arm each time of determination and after the subject has rested in the sitting position (may be supine if during hospitalization) for at least five minutes. Body temperature should be measured as per local practice—the same method to be used consistently for all patients at each site.

### 8.3.3 Height and Weight

Height in centimeters (cm) and body weight (to the nearest 0.1 kilogram [kg]); in indoor clothing, but without shoes will be measured.

### 8.3.4 Laboratory Evaluations

Samples for the laboratory tests noted below will be collected at the time points specified in Schedule of Assessments (see Table 3).

In the case where a laboratory assessment that is listed in the inclusion/exclusion criteria is outside of a protocol-specified range at screening and/or at the initial baseline, the assessment may be repeated once prior to randomization. If the repeat value remains outside of protocol-specified ranges, the subject is excluded from the study.

### 8.3.5 Hematology

Hemoglobin, hematocrit, red blood cell (RBC), MCV, MCH, Platelet Count, white blood cell (WBC) and differential count (% and absolute). At selected major visits HbA1c will also be measured.

### 8.3.6 Clinical Chemistry

Albumin, total bilirubin, calcium, creatinine, glucose, phosphorus, magnesium, potassium, ALT, AST, ALP, GGT, amylase, sodium, bicarbonate, urea/BUN. During weaning and immediately thereafter to Month 24, additional labs should be taken every 2 weeks for ALT, alkaline phosphatase and bilirubine.

(a) Lipid Panel

Cholesterol, triglycerides, LDL, HDL will be measured.

(b) Pregnancy

Serum or urine pregnancy testing will be required for females of child-bearing potential, at timepoints as designated in the Schedule of Assessments (see Table 3). A serum or urine pregnancy test must be obtained up to 72 hours prior to time of transplant and the result must be available and negative prior to administration of investigational product.

(c) Renal Function

Renal function will be assessed by calculated eGFR using the CKD-EPI and MDRD4 formulae.

(d) Coagulation Studies

Coagulation studies will be performed, including international normalized ratio (INR)/prothrombin time (PT) and activated partial thromboplastin time (aPTT).

### 8.3.7 EBV-PTLD Surveillance

Clinical manifestations of EBV infection range from asymptomatic infection to clinically significant and potentially life-threatening disease in transplant recipients. EBV infection can be either primary (new infection occurring in an immunologically naive subject) or secondary due to either reactivation of latent EBV in the transplant recipient under the pressure of immune suppression or reinfection with a new EBV strain. In general, secondary infection tends to be mild or even asymptomatic. Histologic evaluation is important in defining disease status of a subject with suspected PTLD; manifestations can evolve in individual subjects.

### 8.3.8 Imaging: PTLD Surveillance

When warranted based on physical examination findings and/or EBV surveillance, subjects should have an ultrasound performed of the abdominal cavity and allograft to rule out nodal and/or extra-nodal EBV-LPD lesions. CT, MRI and/or PET imaging may be considered for staging and monitoring of biopsy confirmed PTLD.

SEQUENCES

| SEQ | | |
|---|---|---|
| ID | | |
| NO. | DESCRIPTION | SEQUENCE |
| 1 | heavy chain | EYYMY |
| | variable region | |
| | CDR 1 | |
| 2 | heavy chain | RIDPEDGSIDYVEKFKK |
| | variable region | |
| | CDR 2 | |
| 3 | heavy chain | GKFNYRFAY |
| | variable region | |
| | CDR 3 | |
| 4 | light chain | RSSQSLLHSSGNTYLN |
| | variable region | |
| | CDR 1 | |
| 5 | light chain | LVSKLES |
| | variable region | |
| | CDR 2 | |
| 6 | light chain | MQFTHYPYT |
| | variable region | |
| | CDR 3 | |
| 7 | heavy chain | QVQLVQSGAEVQRPGASVKVSCKASGYIFTEYYMYWVRQAPGQG |
| | variable region | LELVGRIDPEDGSIDYVEKFKKKVTLTADTSSSTAYMELSSLTS |
| | | DDTAVYYCARGKENYRFAYWGQGTLVTVSS |
| 8 | light chain | DVVMTQSPPSLLVTLGQPASISCRSSQSLLHSSGNTYLNWLLQR |
| | variable region | PGQSPQPLIYLVSKLESGVPDRFSGSGSGTDFTLKISGVEAEDV |
| | | GVYYCMQFTHYPYTFGQGTKLEIK |
| 9 | heavy chain | QVQLVQSGAEVKKPGASVKVSCKASGYTFTEYYMYWVRQAPGQG |
| | variable region | LELMGRIDPEDGSIDYVEKFKKKVTLTADTSSSTAYMELSSLTS |
| | | DDTAVYYCARGKENYRFAYWGQGTLVTVSS |

What is claimed is:

1. A method of transplantation, the method comprising (i) transplanting a donor liver into a human recipient; and (ii) administering to the recipient an anti-CD2 antibody.

2. The method of claim 1, wherein the anti-CD2 antibody comprises:
(a) a heavy chain variable region CDR 1 of SEQ ID NO:1;
(b) a heavy chain variable region CDR 2 of SEQ ID NO:2;
(c) a heavy chain variable region CDR 3 of SEQ ID NO:3;
(d) a light chain variable region CDR 1 of SEQ ID NO:4;
(e) a light chain variable region CDR 2 of SEQ ID NO: 5; and
(f) a light chain variable region CDR 3 of SEQ ID NO:6.

3. The method of claim 1 or 2, wherein the antibody is a humanized antibody.

4. The method of claim 1 or 2, wherein the antibody is siplizumab.

5. The method of any one of claims 1-4, wherein the anti-CD2 antibody is administered to the recipient at least once within two weeks after the transplant.

6. The method of any one of claims 1-5, wherein the anti-CD2 antibody is administered to the recipient on the day of the transplant, on day 1 after the transplant and on day 4 after the transplant.

7. The method of any one of claims 1-6 wherein the anti-CD2 antibody is administered to the recipient at a dose of 0.1-5 mg/kg/dose

8. The method of any one of claims 1-7 wherein the anti-CD2 antibody is administered to the recipient at a dose of about 0.6 mg/kg/dose.

9. The method of any one of claims 1-8, wherein the anti-CD2 antibody is administered to the recipient intravenously or subcutaneously.

10. The method of any one of claims 1-9, wherein the recipient has undergone a splenectomy.

11. The method of any one of claims 1-9, wherein the recipient has not undergone a splenectomy.

12. The method of any one of claims 1-11, wherein the method further comprises administering cyclophosphamide to the recipient.

13. The method of claim 12, wherein the cyclophosphamide is administered to the recipient at least once during the 30 days after the transplant.

14. The method of claim 12 or 13, wherein the cyclophosphamide is administered to the recipient on day 5 after the transplant.

15. The method of any one of claims 12-14 wherein the cyclophosphamide is administered to the recipient at a dose of 20-100 mg/kg/dose.

16. The method of any one of claims 12-15 wherein the cyclophosphamide is administered to the recipient at a dose of about 40 mg/kg/dose.

17. The method of any one of claims 12-15 wherein the cyclophosphamide is administered to the recipient intravenously.

18. The method of any one of claims 1-17, wherein the method further comprises administering a calcineurin inhibitor to the recipient.

19. The method of claim 18, wherein the calcineurin inhibitor is administered to the recipient once day or twice a day.

20. The method of claim 18 or 19, wherein the calcineurin inhibitor is administered to the recipient within 24 hours of liver reperfusion.

21. The method of claim 18, wherein the calcineurin inhibitor is administered to the recipient for at least 1 month, 2 months, 3 months, 4 months, 5 months, 6 months, 7 months, 8 months, 9 months, 10 months, 11 months, 12 months, 13 months, or at least 14 months post-transplant.

22. The method of claim 18 or 21, wherein the calcineurin inhibitor is administered to the recipient at an amount sufficient to maintain serum through concentrations in the range of 2-15 ng/mL.

23. The method of any one of claims 18-22, wherein the calcineurin inhibitor is administered to the recipient at an amount sufficient to maintain serum through concentrations in the range of 4-11 ng/mL.

24. The method of any one of claims 18-23, wherein the calcineurin inhibitor is administered to the recipient orally.

25. The method of any one of claims 18-22, wherein the calcineurin inhibitor is administered to the recipient intravenously.

26. The method of any one of claims 18-25, wherein the recipient is gradually weaned off the calcineurin inhibitor over 3-24 months after transplantation.

27. The method of any one of claims 18-26, wherein the recipient is gradually weaned off the calcineurin inhibitor over 6-18 months after transplantation.

28. The method of any one of claims 18-27, wherein the dose of the calcineurin inhibitor is reduced to three quarters of the daily dose once a day at six months after transplantation.

29. The method of claim 28, wherein the dosing frequency of the calcineurin inhibitor is reduced three times a week at nine months after transplantation.

30. The method of claim 29, wherein the dosing frequency of the calcineurin inhibitor is further reduced twice a week at twelve months after transplantation.

31. The method of claim 30, wherein the dosing frequency of the calcineurin inhibitor is further reduced to once a week at 15 months after transplantation.

32. The method of claim 31, wherein the administration of the calcineurin inhibitor is stopped 18 months after transplantation.

33. The method of any one of claims 18-32, wherein the calcineurin inhibitor is tacrolimus.

34. The method of any one of claims 1-33, wherein the method further comprises administering steroids to the recipient.

35. The method of claim 34, wherein the steroid is prednisone.

36. The method of claim 34 or 35, wherein the steroid is administered to the recipient on the day of the transplant.

37. The method of any one of claims 34-36, wherein the steroid is administered at a dose of 500-1500 mg.

38. The method of any one of claims 34-37, wherein the steroid is administered at a dose of 1000 mg.

39. The method of any one of claims 34-38, wherein the steroid is administered from day 2 after the transplant through three months after the transplant.

40. The method of any one of claims 34-39, wherein the steroid is administered from day 2 after the transplant through one month after the transplant.

41. The method of any one of claims 34-40, wherein the steroid is administered orally at a dose of 250 mg/day on day 2, a dose of 125 mg/day on day 3, a dose of 75 mg/day on day 4, and at a dose of 60 mg/day from day 5 through to one month after transplant.

42. The method of any one of claims 1-41, wherein the method further comprises administering standard-of-care antimetabolite therapy to the recipient.

43. The method of claim 42, wherein the standard-of-care antimetabolite therapy is administered twice a day for a daily dose of 500-1500 mg/day.

44. The method of claim 42 or 43, wherein the standard-of-care antimetabolite therapy is administered no later than 24 hours after graft reperfusion.

45. The method of any one of claims 42-44, wherein the standard-of-care antimetabolite therapy administration is stopped within six months post-transplant.

46. The method of any one of claims 42-44, wherein the standard-of-care antimetabolite therapy administration is stopped one month post-transplant.

47. The method of any one of claims 42-46, wherein standard-of-care antimetabolite therapy is mycophenolate.
